(19) United States
(12) Patent Application Publication
(10) Pub. No.: US 2021/0220328 A1
(43) Pub. Date: Jul. 22, 2021

(54) PYRROLE DERIVATIVES AS ACC INHIBITORS

(71) Applicant: Almirall, S.A., Barcelona (ES)

(72) Inventors: Jordi BACH TAÑA, Barcelona (ES); Cristina ESTEVE TRIAS, Barcelona (ES); Marta MIR CEPEDA, Barcelona (ES)

(21) Appl. No.: 16/768,940

(22) PCT Filed: Dec. 7, 2018
(86) PCT No.: PCT/EP2018/084039

§ 371 Date: Jun. 2, 2020

(30) Foreign Application Priority Data
Dec. 11, 2017 (EP) 17380025.1

(51) Int. Cl. A61K 31/40; C07D 207/34; A61K 31/4025; C07D 405/06; A61K 31/573; A61K 31/593; A61K 31/203; A61K 39/395; A61K 38/17; A61K 31/20

(52) U.S. Cl. CPC A61K 31/40; C07D 207/34; A61K 31/4025; C07D 405/06; A61K 31/573; A61K 31/20; A61K 31/203; A61K 39/3955; A61K 38/1793; A61K 39/39566; A61K 31/593

(57) ABSTRACT

Novel pyrrole derivatives of Formula (I) are disclosed; as well as process for their preparation, pharmaceutical compositions comprising them and their use in therapy as inhibitors of Acetyl-CoA carboxylase (ACC).

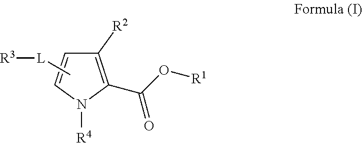

### FIELD OF THE INVENTION

The present invention relates to novel compounds having ACC inhibitory activity. This invention also relates to pharmaceutical compositions containing them, processes for their preparation and their use in the treatment of several disorders.

### BACKGROUND OF THE INVENTION

Acetyl-CoA carboxylase (ACC) is the rate-limiting enzyme in de novo synthesis of fatty acids (Strable M S and Ntambi J M. Crit Rev Biochem Mol Biol. 2010; 45:199-214) and in the translocation of fatty acids to the mitochondria for 3-oxidation (Schreurs M et al. Obes Rev. 2010; 11:380-8). ACC is also key for the elongation of fatty acids including essential fatty acids (Kim C W et al. Cell Metab. 2017; 26:394-406). ACC catalyzes the ATP-dependent carboxylation of acetyl-CoA to malonyl-CoA (Barber M C et al. Biochim Biophys Acta. 2005 March; 1733:1-28). In mammals ACC activity is produced by 2 isoenzymes, namely ACC1 (also known as ACCα) and ACC2 (also known as ACCβ) encoded by 2 different genes (Acc1 and Acc2 respectively) (Barber M C et al. Biochim Biophys Acta. 2005 March; 1733:1-28). ACC1 is located in the cytosol and is involved in the synthesis and elongation of fatty acids. ACC2 is located in cytosolic face of the external mitochondrial membrane and is involved in the inhibition of the carnitine palmitolyltransferase I (CPT-I), which is the crucial enzyme for the transport of long-chain fatty acids to mitochondria for β-oxidation (Tong L. Cell Mol Life Sci. 2013; 70:863-91). The activity of both ACC1 and ACC2 in mammals is stimulated by citrate, inhibited by long chain saturated acyl-CoA, and inactivated by phosphorylation, especially by AMP-activated protein kinase (AMPK) and cAMP-dependent protein kinase (PKA) (Brownsey R W et al. Biochem Soc Trans. 2006; 34:223-7). ACC activity is also key for the survival of several organisms, some of them related to human pathologies such as bacteria, virus and parasites (Tong L. Cell Mol Life Sci. 2013; 70:863-91). In several immune cells types, including T cells and macrophages ACC activity is required for the differentiation, survival and production of cytokines such as IL-17 (Buck M. et al. Cell. 2017; 169:570-86). The crucial role of ACC enzymes in several (patho)physiological processes make them attractive pharmaceutical targets for diseases related to fatty acid metabolism alterations, dermatological diseases such as acne or psoriasis, diabetes, obesity, nonalcoholic steatohepatitis (NASH), cancer, atherosclerosis, inflammation, autoimmunity, infection, and infestation among others (Luo D. et al. Recent Pat Anticancer Drug Discov 2012; 7:168-84). Indeed, several dermatological diseases are linked to ACC activity, for instance acne is characterized for an increase in sebum production (Pappas A. et al. Dermatoendocrinol. 2009; 1:157-61; Williams H et. al. Lancet. 2012; 379:361-72) and both T cells and IL-17 are increased in acne and psoriatic lesions (AgakG. et al. J. Invest. Dermatol. 2014; 134:366-73; Greb J. et al. Nat Rev Dis Primers. 2016; 2:1-17). In acne overactivation of the sebaceous glands leading to the increase in sebum production is a well-known feature of this disease. Sebum is formed mainly from lipids such as triglycerides (TAG), free fatty acids, wax esters, squalene, cholesterol and cholesterol esters. Human sebum is formed mainly from lipids derived from fatty acids such as TAGs and wax esters (Pappas A. Dermatoendocrinol. 2009; 1:72-6) and it has been shown that in humans most of the sebum is produced from de novo synthesis of fatty acids, process that is dependent of ACC activity (Esler W. P et al. WO2015/036892). Both T cells and IL-17 are increased in acne lesions and Th17 cells depend of ACC-mediated fatty acid synthesis for several functions such as the activity of the Th17 master gene RORγt and the production of pro-inflammatory cytokines such as IL-17 (Stokinger B. and Omenetti S. Nat. Rev. Immunol. 2017; 17:535-44). Current acne treatments can be classified between topical and systemic. Topical therapies include retinoids such as adapalene, tretinoin and tazarotene, benzoyl peroxide (BPO) and antibiotics. BPO and retinoids induce skin irritation which can compromise both treatment adherence and efficacy. Topical antibiotics have limited efficacy and are associated to antibiotic resistance. The most efficacious systemic treatments are oral isotretinoin and oral antibiotics (Savage L. and Layton A. Expert Rev Clin Pharmacol. 2010; 13:563-80). Oral isotretinoin treatment is linked to severe side effects including teratogenesis and alteration of blood lipids among others (Layton A. Dermatoendocrinol. 2009; 1:162-9) and oral antibiotics can induce antibiotic resistance. Genetic and pharmacological evidences have shown that ACC inhibitors are useful to reduce sebum production and block IL-17 expression. However no ACC inhibitor has been approved for dermatological indications yet and the only ACC inhibitor currently in development for a dermatologic indication (Olumacostat Glasaretil for acne) has shown a low potency inhibiting sebum production by sebocytes and a poor activity in an in vivo model of sebaceous gland activity (Hunt D. et al. J Invest Dermatol. 2017; 137:1415-23).

In view of the numerous conditions that are contemplated to benefit from treatment involving modulation of the ACC pathway or of the AC carboxylase it is immediately apparent that new compounds that modulate ACC pathways and use of these compounds should provide substantial therapeutic benefits to a wide variety of patients.

Provided herein are novel pyrrole derivatives for use in the treatment of conditions in which targeting of the ACC pathway or inhibition of AC carboxylase can be therapeutically useful.

It has now been found that certain pyrrole derivatives are novel and potent ACC inhibitors and can therefore be used in the treatment or prevention of these diseases.

### SUMMARY OF THE INVENTION

Thus the present invention is directed to new compounds that possess ACC inhibitory activity. Accordingly there is provided a pyrrole derivative, which pyrrole derivative is a compound of Formula (I), or a pharmaceutically acceptable salt, or a solvate, or a N-oxide, or a tautomer, or a stereoisomer, or an isotopically-labelled derivative thereof:

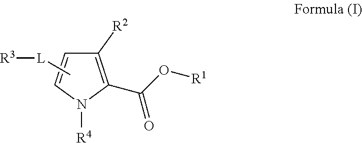

wherein:
R¹ is selected from the group consisting of a hydrogen atom, a linear or branched C₁₋₄ alkyl group, a linear or branched C₁₋₄ haloalkyl group, a linear or branched C₁₋₁₀ hydroxyalkyl group, a —(CH₂)₀₋₃—(C₃₋₇ monocyclic cycloalkyl group), a —(CH₂)₀₋₃-(monocyclic or bicyclic C₆₋₁₄ aryl group), a —(CH₂)₀₋₃-(4- to 7-membered heterocyclyl group containing at least one heteroatom selected from N, O and S), a —(CH₂)₀₋₃— (monocyclic or bicyclic 5- to 14-membered heteroaryl group containing at least one heteroatom selected from N, O and S), a —(CH₂)₀₋₄—[(CH₂)₁₋₃—O]₁₋₅—R^{a} group, a —(CR^{a}R^{b})₁₋₃—OC(O)—R⁵ group and a —(CH₂)₁₋₃—C(O)NR⁵R^{a} group,
   wherein the cycloalkyl, aryl, heterocyclyl and heteroaryl groups are unsubstituted or substituted by one or more substituents selected from a halogen atom, a linear or branched C₁₋₄ alkyl group and an oxo group;
R² is selected from the group consisting of a hydrogen atom, halogen atom, a —CN group and a linear or branched C₁₋₄alkyl group;
R³ represents a linear or branched C₉₋₂₀alkyl group,
   wherein the alkyl group is unsubstituted or substituted by one or more substituents selected from a halogen atom, a hydroxyl group, a linear or branched C₁₋₄ alkyl group, a linear or branched C₁₋₆ alkoxy group and a linear or branched C₁₋₄ hydroxyalkyl group;
R⁴ is selected from the group consisting of a hydrogen atom and a linear or branched C₁₋₄ alkyl group;
R⁵ is selected from the group consisting of a hydrogen atom, a linear or branched C₁₋₁₀ alkyl group, a —O-(linear or branched C₁₋₁₀alkyl group), a —O—(CH₂)₀₋₃—(C₃₋₇ monocyclic cycloalkyl group), a —O—(CH₂)₀₋₃-(monocyclic or bicyclic C₆₋₁₄ aryl group), a —(CH₂)₀₋₃C(O)OR^{a} group and a —O—[(CH₂)₁₋₃—O]₁₋₅—R^{a} group;
   wherein the alkyl group is unsubstituted or substituted by one or more substituents selected from a halogen atom, hydroxyl group and an amino group;
R^{a} and R^{b} are independently selected from the group consisting of a hydrogen atom and a linear or branched C₁₋₄alkyl group; wherein the alkyl group is unsubstituted or substituted by one or more substituents selected from a halogen atom and hydroxyl group; and
L represents a direct bond, a —(CH₂)₀₋₄—O— group, a —(CH₂)₀₋₄—S— group, a —(CH₂)₀₋₄—NRₐ— group, a —C(O)NR^{a}— group, a —NR^{a}C(O)— group or a carbonyl group; characterised in that when R² represents a hydrogen atom, L represents a —(CH₂)₀₋₄—O— group or a —C(O)NR^{a}— group.

The invention further provides synthetic processes and intermediates described herein, which are useful for preparing said pyrrole derivatives.

The invention is also directed to a pyrrole derivative of the invention as described herein for use in the treatment of the human or animal body by therapy.

The invention also provides a pharmaceutical composition comprising the pyrrole derivatives of the invention and a pharmaceutically-acceptable diluent or carrier.

The invention is also directed to the pyrrole derivatives of the invention as described herein, for use in the treatment of a pathological condition or disease susceptible to amelioration by inhibition of Acetyl-CoA carboxylase (ACC), in particular wherein the pathological condition or disease is selected from a dermatological disease, an inflammatory or autoimmune-mediated disease and a metabolism/endocrine function disorder. More in particular wherein the pathological condition or disease is selected from acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis; preferably in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis.

The invention is also directed to use of the pyrrole derivatives of the invention as described herein, in the manufacture of a medicament for treatment of a pathological condition or disease susceptible to amelioration by inhibition of Acetyl-CoA carboxylase (ACC), in particular wherein the pathological condition or disease is selected from a dermatological disease, an inflammatory or autoimmune-mediated disease and a metabolism/endocrine function disorder. More in particular wherein the pathological condition or disease is selected from acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis; preferably in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis.

The invention also provides a method of treatment of a pathological condition or disease susceptible to amelioration by inhibition of Acetyl-CoA carboxylase (ACC), in particular wherein the pathological condition or disease is selected from a dermatological disease, an inflammatory or autoimmune-mediated disease and a metabolism/endocrine function disorder. More in particular wherein the pathological condition or disease is selected from acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis; preferably in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis.

The invention also provides a combination product comprising (i) the pyrrole derivatives of the invention as described herein; and (ii) one or more additional active substances.

### DETAILED DESCRIPTION OF THE INVENTION

When describing the pyrrole derivatives, compositions, combinations and methods of the invention, the following terms have the following meanings, unless otherwise indicated.

As used herein the term C₁₋₁₀ alkyl embraces linear or branched radicals having 1 to 10 carbon atoms. Examples include methyl, ethyl, n-propyl, i-propyl, n-butyl, i-butyl, s-butyl, f-butyl, n-pentyl, 1-methylbutyl, 2-methylbutyl, isopentyl, 1-ethylpropyl, 1,1-dimethylpropyl, 1,2-dimethylpropyl, n-hexyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 2-methylpentyl, 3-methylpentyl, iso-hexyl, heptyl, octyl, nonyl and decyl radicals. Such alkyl radical is typically unsubstituted or substituted by 1, 2 or 3 substituents which may be the same or different.

As used herein the term C₁₋₄alkyl embraces unsubstituted or substituted, linear or branched radicals having 1 to 4 carbon atoms. Analogously, the term C₁₋₃ alkyl embraces linear or branched radicals having 1 to 3 carbon atoms and the term C₁₋₂ alkyl embraces linear or branched radicals having 1 to 2 carbon atoms. Analogously, the term C₂₋₄ alkyl embraces linear or branched radicals having 2 to 4 carbon atoms. Examples of C₁₋₄alkyl include methyl, ethyl, n-propyl, i-propyl, n-butyl, i-butyl, sec-butyl or f-butyl. Such alkyl radical is typically unsubstituted or substituted by 1, 2 or 3 substituents which may be the same or different. Unless otherwise specified, the C₁₋₄alkyl is typically unsubstituted.

As used herein the term C₉₋₂₀ alkyl embraces linear or branched radicals having 9 to 20 carbon atoms. Analogously, the term C₁₀₋₁₇ alkyl embraces linear or branched radicals having 10 to 17 carbon atoms. Examples of C₉₋₂₀alkyl include nonyl, decyl, undecyl, dodecyl, tridecyl, tetradecyl, pentadecyl, hexadecyl, heptadecyl, octadecyl, nonadecyl, icosyl, 3,3-dimethylundecyl, 2,2-dimethyldodecyl and 2,2-dimethyltridecyl radicals. Such alkyl radical is typically unsubstituted or substituted by 1, 2 or 3 substituents which may be the same or different.

As used herein, the term C₁₋₄haloalkyl is a linear or branched alkyl group, which is substituted by one or more, preferably 1, 2 or 3 halogen atoms. Analogously, the term C₁₋₃ haloalkyl is a linear or branched alkyl group, which is substituted by one or more, preferably 1, 2 or 3 halogen atoms. Examples of haloalkyl groups include CCl₃, CF₃, CHF₂, CH₂CF₃ and CH₂CHF₂.

As used herein, the term C₁₋₁₀ hydroxyalkyl embraces linear or branched alkyl radicals having 1 to 10 carbon atoms, any one of which may be substituted with one or more hydroxyl radicals.

Analogously, the term C₂₋₁₀ hydroxyalkyl embraces linear or branched alkyl radicals having 2 to 10 carbon atoms, any one of which may be substituted with one or more hydroxyl radicals and the term C₃₋₉ hydroxyalkyl embraces linear or branched alkyl radicals having 3 to 9 carbon atoms, any one of which may be substituted with one or more hydroxyl radicals. Examples of such radicals include hydroxymethyl, hydroxyethyl, hydroxypropyl, hydroxybutyl, hydroxypentyl, hydroxyhexyl, hydroxyheptyl, hydroxyoctyl, hydroxynonyl, hydroxydecyl, 2,3-dihydroxypropyl and 1,3-dihydroxypropan-2-yl.

As used herein, the term C₁₋₄ hydroxyalkyl embraces linear or branched alkyl radicals having 1 to 4 carbon atoms, any one of which may be substituted with one or more hydroxyl radicals. Examples of such radicals include hydroxymethyl, hydroxyethyl, hydroxypropyl or hydroxybutyl.

As used herein, the term C₁-C₆ alkoxy (or alkyloxy) embraces linear or branched oxy-containing radicals each having alkyl portions of 1 to 6 carbon atoms. Examples of C₁-C₆ alkoxy radicals include methoxy, ethoxy, n-propoxy, i-propoxy, n-butoxy, i-butoxy, sec-butoxy, f-butoxy, n-pentoxy and n-hexoxy.

As used herein, the term C₁-C₃ alkoxy (or alkyloxy) embraces linear or branched oxy-containing radicals each having alkyl portions of 1 to 3 carbon atoms. Examples of C₁-C₃ alkoxy radicals include methoxy, ethoxy, n-propoxy and i-propoxy.

As used herein, the term monocyclic C₃₋₇ cycloalkyl embraces saturated monocyclic carbocyclic radicals having from 3 to 7 carbon atoms. Examples of monocyclic C₃₋₇ cycloalkyl groups include cyclopropyl, cyclobutyl, cyclopentyl, cyclohexyl and cycloheptyl. Such C₃₋₇ cycloalkyl radical is typically unsubstituted or substituted by 1, 2 or 3 substituents which may be the same or different.

As used herein, the term monocyclic or bicyclic C₆₋₁₄ aryl radical embraces typically a C₆₋₁₄, more preferably C₆₋₁₀ monocyclic or bicyclic aryl radical such as phenyl, naphthyl, anthranyl and phenanthryl. Phenyl is preferred. Such C₆₋₁₄ aryl radical is typically unsubstituted or substituted by 1, 2 or 3 substituents which may be the same or different.

As used herein, the term 4- to 7-membered heterocyclyl radical embraces typically a non-aromatic, saturated or unsaturated C₄₋₇ carbocyclic ring system in which one or more, for example 1, 2, 3 or 4 of the carbon atoms, preferably 1 or 2 of the carbon atoms, are replaced by a heteroatom selected from N, O and S. Examples of 4- to 7-membered heterocyclyl radicals include oxetanyl, azetidinyl, piperidyl, pyrrolidyl, pyrrolinyl, piperazinyl, morpholinyl, thiomorpholinyl, pyrrolyl, pyrazolinyl, pirazolidinyl, triazolyl, pyrazolyl, tetrazolyl, imidazolidinyl, 4,5-dihydro-oxazolyl, 1,3-dioxol-2-one, tetrahydrofuranyl, 3-aza-tetrahydrofuranyl, tetrahydrothiophenyl, tetrahydropyranyl, tetrahydrothiopyranyl, 1,4-azathianyl, 2,5-dioxopyrrolidinyl, 2-oxopyrrolidinyl), 1,3-dioxol-4-yl or 1,3-dioxolyl. Such heterocyclyl radical is typically unsubstituted or substituted by 1, 2 or 3 substituents which may be the same or different. Analogously, term 5- to 6-membered heterocyclyl radical embraces typically a non-aromatic, saturated or unsaturated C₅₋₆ carbocyclic ring system in which one or more, for example 1, 2, 3 or 4 of the carbon atoms, preferably 1 or 2 of the carbon atoms, are replaced by a heteroatom selected from N, O and S. Examples of 5- to 6-membered heterocyclyl radicals include piperidyl, pyrrolidyl, pyrrolinyl, piperazinyl, morpholinyl, thiomorpholinyl, pyrrolyl, pyrazolinyl, pirazolidinyl, triazolyl, pyrazolyl, tetrazolyl, imidazolidinyl, 4,5-dihydro-oxazolyl, 1,3-dioxol-2-one, tetrahydrofuranyl, 3-aza-tetrahydrofuranyl, tetrahydrothiophenyl, tetrahydropyranyl, tetrahydrothiopyranyl, 1,4-azathianyl, 2,5-dioxopyrrolidinyl, 2-oxopyrrolidinyl, 1,3-dioxol-4-yl or 1,3-dioxolyl. As used herein, the term monocyclic or bicyclic 5- to 14-membered heteroaryl radical embraces typically a 5- to 14-membered ring system, comprising at least one heteroaromatic ring and containing at least one heteroatom selected from O, S and N, preferably S and N. A 5- to 14-membered heteroaryl radical may be a single ring or two fused rings wherein at least one ring contains a heteroatom. Examples include pyridyl, pyrazinyl, pyrimidinyl, pyridazinyl, furyl, benzofuranyl, oxadiazolyl, oxazolyl, isoxazolyl, benzoxazolyl, imidazolyl, benzimidazolyl, thiazolyl, thiadiazolyl, thienyl, pyrrolyl, benzo[b]thienyl, benzothiazolyl, indolyl, indazolyl, purinyl, quinolinyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl, thianthrenyl, pyrazolyl, 2H-pyrazolo[3,4-tf]pyrimidinyl, 1H-pyrazolo[3,4-cf]pyrimidinyl, benzo[b]thienyl, thieno[2,3-d] pyrimidinyl, thieno[3,2-cf]pyrimidinyl and the various pyrrolopyridyl, pyridopyrimidinyl, pyrimidopyridazinyl, pyrazinopyrimidinyl, imidazotriazinyl, pyridotriazinyl and triazolopyrimidinyl radicals.

As used herein, the term halogen atom embraces chlorine, fluorine, bromine and iodine atoms. A halogen atom is typically a fluorine, chlorine or bromine atom. The term halo when used as a prefix has the same meaning.

As used herein, the term carbonyl group refers to a —C(O)— moiety [i.e. a bivalent moiety comprising a carbon atom attached to an oxygen atom via a double bond].

As used herein, the term oxo group refers to a ═O moiety [i.e. a substituent oxygen atom connected to another atom via a double bond].

As used herein, some of the atoms, radicals, moieties, chains and cycles present in the general structures of the invention are “unsubstituted or substituted”. This means that these atoms, radicals, moieties, chains and cycles can be either unsubstituted or substituted in any position by one or more, for example 1, 2, 3 or 4, substituents, whereby the hydrogen atoms bound to the unsubstituted atoms, radicals, moieties, chains and cycles are replaced by chemically acceptable atoms, radicals, moieties, chains and cycles.

Compounds containing one or more chiral centre may be used in enantiomerically or diastereoisomerically pure form, in the form of racemic mixtures and in the form of mixtures enriched in one or more stereoisomer. The scope of the invention as described and claimed encompasses the racemic forms of the compounds as well as the individual enantiomers, diastereomers, and stereoisomer-enriched mixtures.

Conventional techniques for the preparation/isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomer conglomerates may be separated by conventional techniques known to those skilled in the art. See, e.g. “Stereochemistry of Organic Compounds” by Ernest L. Eliel (Wiley, New York, 1994).

The term “therapeutically effective amount” refers to an amount sufficient to effect treatment when administered to a patient in need of treatment.

The term “treatment” as used herein refers to the treatment of a disease or medical condition in a human patient which includes:

(a) preventing the disease or medical condition from occurring, i.e., prophylactic treatment of a patient;

(b) ameliorating the disease or medical condition, i.e., causing regression of the disease or medical condition in a patient;

(c) suppressing the disease or medical condition, i.e., slowing the development of the disease or medical condition in a patient; or

(d) alleviating the symptoms of the disease or medical condition in a patient.

The phrase “pathological condition or disease susceptible to amelioration by inhibition ACC” includes all disease states and/or conditions that are acknowledged now, or that are found in the future, to be associated with an increased ACC activity. Such disease states include, but are not limited to, dermatological diseases, inflammatory or autoimmune-mediated diseases and a metabolism/endocrine function disorders.

As used herein, the term “pharmaceutically acceptable salt” refers to a salt prepared from a base or acid which is acceptable for administration to a patient, such as a mammal. Such salts can be derived from pharmaceutically-acceptable inorganic or organic bases and from pharmaceutically-acceptable inorganic or organic acids.

As used herein, a N-oxide is formed from the tertiary basic amines or imines present in the molecule, using a convenient oxidising agent.

The pyrrole derivatives of the invention may exist in both unsolvated and solvated forms. The term solvate is used herein to describe a molecular complex comprising a compound of the invention and an amount of one or more pharmaceutically acceptable solvent molecules. The term hydrate is employed when said solvent is water. Examples of solvate forms include, but are not limited to, compounds of the invention in association with water, acetone, dichloromethane, 2-propanol, ethanol, methanol, dimethylsulfoxide (DMSO), ethyl acetate, acetic acid, ethanolamine, or mixtures thereof.

The invention also includes isotopically-labelled pyrrole derivatives of the invention, wherein one or more atoms is replaced by an atom having the same atomic number, but an atomic mass or mass number different from the atomic mass or mass number usually found in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Preferred isotopically-labelled compounds include deuterated derivatives of the compounds of the invention. As used herein, the term deuterated derivative embraces compounds of the invention where in a particular position at least one hydrogen atom is replaced by deuterium. Deuterium (D or ²H) is a stable isotope of hydrogen which is present at a natural abundance of 0.015 molar %.

Isotopically-labelled pyrrole derivatives of the invention can generally be prepared by conventional techniques known to those skilled in the art or by processes analogous to those described herein, using an appropriate isotopically-labelled reagent in place of the non-labelled reagent otherwise employed.

As used in the present invention, the term tautomer means two or more forms or isomers of an organic compound that readily could be interconverted into each other via a common chemical reaction called tautomerization. This reaction commonly results in the formal migration of a hydrogen atom or proton, accompanied by a switch of a single bond and adjacent double bond. The concept of tautomerizations is called tautomerism. Because of the rapid interconversion, tautomers are generally considered to be the same chemical compound. In solutions in which tautomerization is possible, a chemical equilibrium of the tautomers will be reached. The exact ratio of the tautomers depends on several factors, including temperature, solvent and pH.

Prodrugs of the pyrrole derivatives described herein are also within the scope of the invention. Thus certain derivatives of the pyrrole derivatives of the present invention, which derivatives may have little or no pharmacological activity themselves, when administered into or onto the body may be converted into compounds of the present invention having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of the present invention with certain moieties known to those skilled in the art as ‘pro-moieties’ as described, for example, in Design of Prodrugs by H. Bundgaard (Elsevier, 1985).

In the case of pyrrole derivatives that are solids, it is understood by those skilled in the art that the inventive compounds and salts may exist in different crystalline or polymorphic forms, or in an amorphous form, all of which are intended to be within the scope of the present invention.

Compounds of Formula (I) may contain more than one Rₐ moiety. When a compound contains more than one Rₐ moiety, each Rₐ moiety may be the same or different.

Compound of Formula (I) contain a bivalent -L- moiety, wherein L is as herein defined. When L represents a —(CH₂)₀₋₄—O— group, a —(CH₂)₀₋₄—S— group, a —(CH₂)₀₋₄—NRₐ— group, a —C(O)NR^{a}— group, a —NR^{a}C(O)— group, the L moiety may be positioned either (a) so that the bond on the left hand side of the L moiety is to the R³ moiety, and the bond on the right hand side of the L moiety is to the central pyrrole ring, or (b) so that the bond on the right hand side of the L moiety is to the R³ moiety, and the bond on the left hand side of the L moiety is to the central pyrrole ring, with orientation (a) generally preferred. For example, in the case of L representing a —(CH₂)₀₋₄—O— group, the —(CH₂)₀₋₄—O— group can be positioned either (a) so that the —(CH₂)₀₋₄ portion is attached to R³ and the O— portion is attached to the central pyrrole ring, or (b) so that the —(CH₂)₀₋₄ portion is attached to the central pyrrole ring and the —O— portion is attached R³.

When R³ represents a linear or branched C₉₋₂₀ alkyl group, which is substituted by one or more substituents selected from a linear or branched C₁₋₄alkyl group, a linear or branched C₁₋₆ alkoxy group and a linear or branched C₁₋₄ hydroxyalkyl group, it is preferred that the total number of carbon atoms in the R³ moiety remains 9-20.

Preferably there is provided a pyrrole derivative, which pyrrole derivative is a compound of Formula (I), or a pharmaceutically acceptable salt, or a solvate, or a N-oxide, or a tautomer, or a stereoisomer, or an isotopically-labelled derivative thereof:

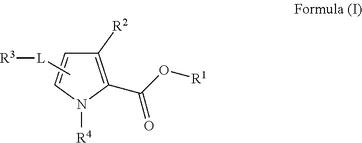

wherein:
R¹ is selected from the group consisting of a hydrogen atom, a linear or branched C₁₋₄ alkyl group, a linear or branched C₁₋₄ haloalkyl group, a linear or branched C₁₋₁₀ hydroxyalkyl group, a —(CH₂)₀₋₃—(C₃₋₇ monocyclic cycloalkyl group), a —(CH₂)₀₋₃-(monocyclic or bicyclic C₆₋₁₄ aryl group), a —(CH₂)₀₋₃-(4- to 7-membered heterocyclyl group containing at least one heteroatom selected from N, O and S), a —(CH₂)₀₋₃— (monocyclic or bicyclic 5- to 14-membered heteroaryl group containing at least one heteroatom selected from N, O and S), a —(CH₂)₀₋₄—[(CH₂)₁₋₃—O]₁₋₅—R^{a} group, a —(CR^{a}R^{b})₁₋₃—OC(O)—R⁵ group and a —(CH₂)₁₋₃—C(O)NR⁵R^{a} group,
   wherein the cycloalkyl, aryl, heterocyclyl and heteroaryl groups are unsubstituted or substituted by one or more substituents selected from a halogen atom, a linear or branched C₁₋₄ alkyl group and an oxo group;
R² is selected from the group consisting of a hydrogen atom, halogen atom and a linear or branched C₁₋₄ alkyl group;
R³ represents a linear or branched C₉₋₂₀ alkyl group,
   wherein the alkyl group is unsubstituted or substituted by one or more substituents selected from a halogen atom, a hydroxyl group, a linear or branched C₁₋₄ alkyl group, a linear or branched C₁₋₆ alkoxy group and a linear or branched C₁₋₄ hydroxyalkyl group;
R⁴ is selected from the group consisting of a hydrogen atom and a linear or branched C₁₋₄ alkyl group;
R⁵ is selected from the group consisting of a hydrogen atom, a linear or branched C₁₋₁₀ alkyl group, a —O-(linear or branched C₁₋₁₀alkyl group), a —O—(CH₂)₀₋₃—(C₃₋₇ monocyclic cycloalkyl group), a —O—(CH₂)₀₋₃-(monocyclic or bicyclic C₆₋₁₄ aryl group), a —(CH₂)₀₋₃C(O)OR^{a} group and a —O—[(CH₂)₁₋₃—O]₁₋₅—R^{a} group;
   wherein the alkyl group is unsubstituted or substituted by one or more substituents selected from a halogen atom, hydroxyl group and an amino group;
R^{a} and R^{b} are independently selected from the group consisting of a hydrogen atom and a linear or branched C₁₋₄alkyl group; wherein the alkyl group is unsubstituted or substituted by one or more substituents selected from a halogen atom and hydroxyl group; and
L represents a direct bond, a —(CH₂)₀₋₄—O— group, a —(CH₂)₀₋₄—S— group, a —(CH₂)₀₋₄—N— group, a —C(O)NR^{a}— group, a —NR^{a}C(O)— group or a carbonyl group; characterised in that when R² represents a hydrogen atom, L represents a —(CH₂)₀₋₄—O— group or a —C(O)NR^{a}— group, and preferably wherein (a) L represents a direct bond, a —(CH₂)₀₋₄—O— group, a —(CH₂)₀₋₄—S— group, a —C(O)NR^{a}— group, a —NR^{a}C(O)— group or a carbonyl group; characterised in that when R² represents a hydrogen atom, L represents a —(CH₂)₀₋₄—O— group or a —C(O)NR^{a}— group, or (b) L represents a direct bond, a —(CH₂)₀₋₄—O— group, a —(CH₂)₀₋₄—S— group, a —(CH₂)₀₋₄—NRₐ— group, a —C(O)NR^{a}— group, a —NR^{a}C(O)— group or a carbonyl group; characterised in that when R² represents a hydrogen atom, L represents a —(CH₂)₀₋₄—O— group or a —C(O)NR^{a}— group.

Typically, the compound of Formula (I) is a compound of Formula (Ia) or a compound of Formula (Ib),

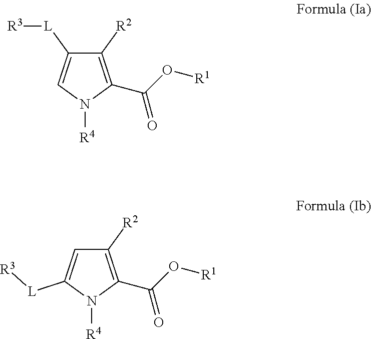

Preferably, the compound of Formula (I) is a compound of Formula (Ia).

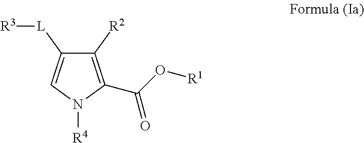

It is also preferred that the compound of Formula (I) is a compound of Formula (Ib).

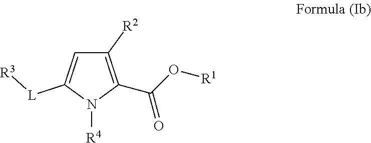

Typically, R¹ is selected from the group consisting of a hydrogen atom, a linear or branched C₁₋₄ alkyl group, a linear or branched C₁₋₄ haloalkyl group, a linear or branched C₂₋₁₀ hydroxyalkyl group, a cyclohexyl group, a —CH₂-phenyl group, a —(CH₂)₁₋₂-(5- to 6-membered heterocyclyl group containing at least one heteroatom selected from N, O and S), a —(CH₂CH₂O)₁₋₄—R^{a} group, a —(CR^{a}R^{b})₁₋₃—OC(O)—R⁵ group and a —(CH₂)₁₋₃—C(O)NR⁵R^{a} group, wherein the cyclohexyl, phenyl and heterocyclyl groups are unsubstituted or substituted by one or more substituents selected from a halogen atom, a linear or branched C₁₋₄alkyl group and an oxo group.

Preferably, R¹ is selected from the group consisting of a hydrogen atom, a linear or branched C₁₋₃ haloalkyl group, a linear or branched C₃₋₉ hydroxyalkyl group, a —(CH₂)₁₋₂-(5-membered heterocyclyl group containing at least one heteroatom selected from N and O), a —(CH₂CH₂O)₂—R^{a} group, a —(CR^{a}R^{b})—OC(O)—R⁵ group and a —(CH₂)—C(O)NR⁵R^{a} group, wherein the heterocyclyl group is unsubstituted or substituted by one or more substituents selected from a linear or branched C₁₋₄ alkyl group and an oxo group.

More preferably, R¹ is selected from the group consisting of a hydrogen atom, a —CH₂CF₃ group, a —(CH₂)₉—OH group, a —CH₂CH(OH)CH₂OH group, a —CH(CH₂OH)₂ group, a —(CH₂)₂-(2,5-dioxopyrrolidin-1-yl) group, a —(CH₂)-(5-methyl-2-oxo-1,3-dioxol-4-yl) group, a —(CH₂CH₂O)₂—R^{a} group, a —(CR^{a}H)₁₋₃—OC(O)—R⁵ group and a —CH₂—C(O)NR⁵R^{a} group,

Typically, R² represents a halogen atom, a methyl group or a hydrogen atom.

Preferably, R² represents a halogen atom.

More preferably, R² represents a fluorine or chlorine atom.

It is also preferred that R² represents a hydrogen atom, methyl group, fluorine atom, chlorine atom or bromine atom.

Typically, R³ represents a linear or branched C₉₋₂₀ alkyl group, wherein the alkyl group is unsubstituted or substituted by one or more substituents selected from a halogen atom, a hydroxyl group, a linear or branched alkyl group and a linear or branched C₁₋₃ alkoxy group.

Preferably, R³ represents a linear or branched C₁₀₋₁₇ alkyl group, wherein the alkyl group is unsubstituted or substituted by one or more substituents selected from a halogen atom, a hydroxyl group, a linear or branched alkyl group and a linear or branched C₁₋₃ alkoxy group.

More preferably, R³ represents a linear or branched C₁₀₋₁₇ alkyl group, wherein the alkyl group is unsubstituted or substituted by one or more substituents selected from a fluorine atom, a linear or branched C₁₋₄ alkyl group and a linear or branched C₁₋₃ alkoxy group.

Even more preferably, R³ represents a linear or branched C₁₀₋₁₇ alkyl group, wherein the alkyl group is unsubstituted or substituted by one or more substituents selected from a fluorine atom, methyl group and ethoxy group.

It is also preferred that R³ represents a linear or branched C₉₋₁₇ alkyl group, wherein the alkyl group is unsubstituted or substituted by one or more substituents selected from a fluorine atom, a linear or branched C₁₋₄ alkyl group and a linear or branched C₁₋₃ alkoxy group.

Typically, R⁴ represents a hydrogen atom and a linear or branched C₁₋₄alkyl group.

Preferably, R⁴ represents a hydrogen atom.

Typically, R⁵ is selected from the group consisting of a —O-(linear or branched C₁₋₁₀ alkyl group), a —O-cyclohexyl group, a —O—CH₂-phenyl group, a —(CH₂)₁₋₂C(O)OR^{a} group, a —O—(CH₂CH₂O)₁₋₃—R^{a} group and a —O—CH₂CH₂CH₂O—R^{a} group.

Preferably, R⁵ is selected from the group consisting of a —O-(linear or branched C₂₋₄ alkyl group), a —O-cyclohexyl group, a —O—CH₂-phenyl group, a —(CH₂)—C(O)OR^{a} group, a —O—(CH₂CH₂O)₁₋₂—R^{a} group and a —O—CH₂CH₂CH₂O—R^{a} group. More preferably, R⁵ is selected from the group consisting of a —O—CH(CH₃)₂ group, a —O—C(CH₃)₃ group, a —O-cyclohexyl group, a —O—CH₂-phenyl group, a —CH₂—C(O)OR^{a} group, a —O—(CH₂CH₂O)₁₋₂—R^{a} group and a —O—CH₂CH₂CH₂O—R^{a} group.

Typically, R^{a} is selected from the group consisting of a hydrogen atom and a linear or branched C₁₋₄alkyl group; wherein the alkyl group is unsubstituted or substituted by one or more substituents selected from a halogen atom and hydroxyl group.

Preferably, R^{a} is selected from the group consisting of a hydrogen atom and a linear or branched C₁₋₄alkyl group; wherein the alkyl group is unsubstituted or substituted by one or more hydroxyl groups.

More preferably, R^{a} is selected from the group consisting of a hydrogen atom and a linear or branched C₁₋₄ alkyl group.

Even more preferably, R^{a} is selected from the group consisting of a hydrogen atom and a linear or branched C₁₋₂ alkyl group.

It is also preferred, that R^{a} represents a hydrogen atom or a linear or branched C₁₋₃ alkyl group; wherein the alkyl group is unsubstituted or substituted by one or more hydroxyl groups.

Typically, R^{b} is selected from the group consisting of a hydrogen atom and a linear or branched C₁₋₄ alkyl group.

Preferably, R^{b} represents a hydrogen atom.

Typically, L represents a direct bond, a —(CH₂)₀₋₄—O— group, or a —(CH₂)₀₋₄—S— group, characterised in that when R² represents a hydrogen atom, L represents a —(CH₂)₀₋₄—O—.

Preferably, L represents a direct bond, —O— or —S—, characterised in that when R² represents a hydrogen atom, L represents —O—.

More preferably, L represents a direct bond or a —(CH₂)₀₋₄—O— group.

Even more preferably, L represents a direct bond or a —(CH₂)₀₋₁—O— group.

Still more preferably, L represents a direct bond or —O—.

It is particularly preferred that L represents a direct bond.

It is also particularly preferred that L represents —O—.

In a particular preferred embodiment, in the compound of formula (I)
R² represents a halogen atom, preferably R² represents a fluorine or chlorine atom;
R³ represents a linear or branched C₉₋₂₀alkyl group,
   wherein the alkyl group is unsubstituted or substituted by one or more substituents selected from a halogen atom, a hydroxyl group, a linear or branched C₁₋₄ alkyl group and a linear or branched C₁₋₃ alkoxy group; and
L represents a direct bond or —O—.

In one embodiment, the compound of Formula (I) is represented by Formula (Ia),

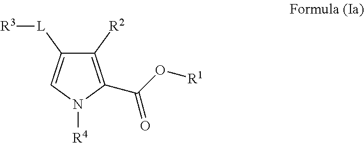

wherein:
R¹ is selected from the group consisting of a hydrogen atom, a linear or branched C₁₋₄ alkyl group, a linear or branched C₁₋₄ haloalkyl group, a linear or branched C₂₋₁₀ hydroxyalkyl group, a cyclohexyl group, a —CH₂-phenyl group, a —(CH₂)₁₋₂-(5- to 6-membered heterocyclyl group containing at least one heteroatom selected from N, O and S), a —(CH₂CH₂O)₁₋₄—R^{a} group, a —(CR^{a}R^{b})₁₋₃—OC(O)—R⁵ group and a —(CH₂)₁₋₃—C(O)NR⁵R^{a} group,
   wherein the cyclohexyl, phenyl and heterocyclyl groups are unsubstituted or substituted by one or more substituents selected from a halogen atom, a linear or branched C₁₋₄ alkyl group and an oxo group;
R² represents a halogen atom;
R³ represents a linear or branched C₁₀₋₁₇ alkyl group, wherein the alkyl group is unsubstituted or substituted by one or more substituents selected from a halogen atom, a hydroxyl group, a linear or branched C₁₋₄ alkyl group and a linear or branched C₁₋₃ alkoxy group;
R⁴ represents a hydrogen atom;
R⁵ is selected from the group consisting of a —O-(linear or branched C₁₋₁₀ alkyl group), a —O-cyclohexyl group, a —O—CH₂-phenyl group, a —(CH₂)₁₋₂C(O)OR^{a} group, a —O—(CH₂CH₂O)₁₋₃—R^{a} group and a —O—CH₂CH₂CH₂O—R^{a} group;
R^{a} is selected from the group consisting of a hydrogen atom and a linear or branched C₁₋₄ alkyl group, wherein the alkyl group is unsubstituted or substituted by one or more substituents selected from a halogen atom and a hydroxyl group;
R^{b} represents a hydrogen atom; and
L represents a direct bond or —O—.

In a preferred embodiment in the compound of formula (Ia),
R¹ is selected from the group consisting of a hydrogen atom, a linear or branched C₁₋₃ haloalkyl group, a linear or branched C₃₋₉ hydroxyalkyl group, a —(CH₂)₁₋₂-(5-membered heterocyclyl group containing at least one heteroatom selected from N and O), a —(CH₂CH₂O)₂—R^{a} group, a —(CR^{a}R^{b})—OC(O)—R⁵ group and a —(CH₂)—C(O)NR⁵R^{a} group,
   wherein the heterocyclyl group is unsubstituted or substituted by one or more substituents selected from a linear or branched C₁₋₄ alkyl group and an oxo group;
R² represents a fluorine atom or a chlorine atom;
R³ represents a linear or branched C₁₀₋₁₇ alkyl group, wherein the alkyl group is unsubstituted or substituted by one or more substituents selected from a fluorine atom, a linear or branched C₁₋₄alkyl group and a linear or branched C₁₋₃alkoxy group;
R⁵ is selected from the group consisting of a —O-(linear or branched C₂₋₄ alkyl group), a —O-cyclohexyl group, a —O—CH₂-phenyl group, a —(CH₂)—C(O)OR^{a} group, a —O—(CH₂CH₂O)₁₋₂—R^{a} group and a —O—CH₂CH₂CH₂O—R^{a} group;
R^{a} is selected from the group consisting of a hydrogen atom and a linear or branched C₁₋₄ alkyl group; wherein the alkyl group is unsubstituted or substituted by one or more substituents selected from a halogen atom and hydroxyl group.

In a still more preferred embodiment, in the compound of formula (Ia),
R¹ is selected from the group consisting of a hydrogen atom, a —CH₂CF₃ group, a —(CH₂)₉—OH group, a —CH₂CH(OH)CH₂OH group, a —CH(CH₂OH)₂ group, a —(CH₂)₂-(2,5-dioxopyrrolidin-1-yl) group, a —(CH₂)-(5-methyl-2-oxo-1,3-dioxol-4-yl) group, a —(CH₂CH₂O)₂—R^{a} group, a —(CR^{a}H)₁₋₃—OC(O)—R⁵ group and a —CH₂—C(O)NR⁵R^{a} group,
R³ represents a linear or branched C₁₀₋₁₇ alkyl group, wherein the alkyl group is unsubstituted or substituted by one or more substituents selected from a fluorine atom, methyl group and ethoxy group;
R⁵ is selected from the group consisting of a —O—CH(CH₃)₂ group, a —O—C(CH₃)₃ group, a —O-cyclohexyl group, a —O—CH₂-phenyl group, a —CH₂—C(O)OR^{a} group, a —O—(CH₂CH₂O)₁₋₂—R^{a} group and a —O—CH₂CH₂CH₂O—R^{a} group;
R^{a} is selected from the group consisting of a hydrogen atom and a linear or branched C₁₋₂ alkyl group.

In one embodiment, the compound of Formula (I) is represented by Formula (Ib),

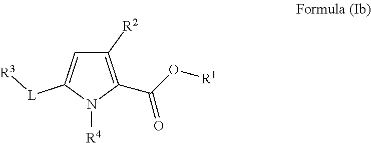

wherein:
R¹ is selected from the group consisting of a hydrogen atom, a linear or branched C₁₋₄ alkyl group, a linear or branched C₁₋₄ haloalkyl group, a linear or branched C₂₋₁₀ hydroxyalkyl group, a cyclohexyl group, a —CH₂-phenyl group, a —(CH₂)₁₋₂-(5- to 6-membered heterocyclyl group containing at least one heteroatom selected from N, O and S), a —(CH₂CH₂O)₁₋₄—R^{a} group, a —(CR^{a}R^{b})₁₋₃—OC(O)—R⁵ group and a —(CH₂)₁₋₃—C(O)NR⁵R^{a} group,
   wherein the cyclohexyl, phenyl and heterocyclyl groups are unsubstituted or substituted by one or more substituents selected from a halogen atom, a linear or branched C₁₋₄ alkyl group and an oxo group;
R² represents a halogen atom;
R³ represents a linear or branched C₁₀₋₁₇ alkyl group, wherein the alkyl group is unsubstituted or substituted by one or more substituents selected from a halogen atom, a hydroxyl group, a linear or branched C₁₋₄ alkyl group and a linear or branched C₁₋₃ alkoxy group;
R⁴ represents a hydrogen atom;
R⁵ is selected from the group consisting of a —O-(linear or branched C₁₋₁₀ alkyl group), a —O-cyclohexyl group, a —O—CH₂-phenyl group, a —(CH₂)₁₋₂C(O)OR^{a} group, a —O—(CH₂CH₂O)₁₋₃—R^{a} group and a —O—CH₂CH₂CH₂O—R^{a} group;
R^{a} is selected from the group consisting of a hydrogen atom and a linear or branched C₁₋₄ alkyl group, wherein the alkyl group is unsubstituted or substituted by one or more substituents selected from a halogen atom and a hydroxyl group;
R^{b} represents a hydrogen atom; and
L represents a direct bond or —O—.

In a preferred embodiment, in the compound of formula (Ib),
R¹ is selected from the group consisting of a hydrogen atom, a linear or branched C₁₋₃ haloalkyl group, a linear or branched C₃₋₉ hydroxyalkyl group, a —(CH₂)₁₋₂-(5-membered heterocyclyl group containing at least one heteroatom selected from N and O), a —(CH₂CH₂O)₂—R^{a} group, a —(CR^{a}R^{b})—OC(O)—R⁵ group and a —(CH₂)—C(O)NR⁵R^{a} group,
   wherein the heterocyclyl group is unsubstituted or substituted by one or more substituents selected from a linear or branched C₁₋₄ alkyl group and an oxo group;
R² represents a fluorine atom or a chlorine atom;
R³ represents a linear or branched C₁₀₋₁₇ alkyl group, wherein the alkyl group is unsubstituted or substituted by one or more substituents selected from a fluorine atom, a linear or branched C₁₋₄alkyl group and a linear or branched C₁₋₆ alkoxy group;
R⁵ is selected from the group consisting of a —O-(linear or branched C₂₋₄ alkyl group), a —O-cyclohexyl group, a —O—CH₂-phenyl group, a —(CH₂)—C(O)OR^{a} group, a —O—(CH₂CH₂O)₁₋₂—R^{a} group and a —O—CH₂CH₂CH₂O—R^{a} group;
R^{a} is selected from the group consisting of a hydrogen atom and a linear or branched C₁₋₄ alkyl group; wherein the alkyl group is unsubstituted or substituted by one or more substituents selected from a halogen atom and a hydroxyl group.

In a more preferred embodiment, in the compound of formula (Ib),
R¹ is selected from the group consisting of a hydrogen atom, a —CH₂CF₃ group, a —(CH₂)₉—OH group, a —CH₂CH(OH)CH₂OH group, a —CH(CH₂OH)₂ group, a —(CH₂)₂-(2,5-dioxopyrrolidin-1-yl) group, a —(CH₂)-(5-methyl-2-oxo-1,3-dioxol-4-yl) group, a —(CH₂CH₂O)₂—R^{a} group, a —(CR^{a}H)₁₋₃—OC(O)—R⁵ group and a —CH₂—C(O)NR⁵R^{a} group,
R³ represents a linear or branched C₁₀₋₁₇ alkyl group, wherein the alkyl group is unsubstituted or substituted by one or more substituents selected from a fluorine atom, methyl group and ethoxy group;
R⁵ is selected from the group consisting of a —O—CH(CH₃)₂ group, a —O—C(CH₃)₃ group, a —O-cyclohexyl group, a —O—CH₂-phenyl group, a —CH₂—C(O)OR^{a} group, a —O—(CH₂CH₂O)₁₋₂—R^{a} group and a —O—CH₂CH₂CH₂O—R^{a} group;
R^{a} is selected from the group consisting of a hydrogen atom and a linear or branched C₁₋₂ alkyl group.

In one embodiment, in the compound of formula (I),
R¹ is selected from the group consisting of a hydrogen atom, a linear or branched C₁₋₄ alkyl group, a —CH₂CF₃ group, a —(CH₂)₂₋₉—OH group, a —CH₂—CH(OH)—CH₂—OH, a —CH(CH₂OH)₂ group, a cyclohexyl group, a —(CH₂)₂-(2,5-dioxopyrrolidin-1-yl) group, a —(CH₂)₂-(2-oxopyrrolidin-1-yl) group, a —(CH₂)-(5-methyl-2-oxo-1,3-dioxol-4-yl) group, a —CH₂-phenyl group, a —(CH₂CH₂O)₂₋₄—R^{a} group, a —CH(CH₃)—OC(O)OCH(CH₃)₂ group, a —CH(CH₃)—OC(O)OC(CH₃)₃ group, a —CH(CH₃)—OC(O)O(CH₂)₈CH₃ group, a —CH(CH₃)—OC(O)O-cyclohexyl group, a —CH(CH₃)—OC(O)O—CH₂-phenyl group, a —CH(CH₃)—OC(O)O(CH₂CH₂O)₁₋₂—R^{a} group, a —CH(CH₃)—OC(O)O(CH₂)₃OH group, a —(CH₂)₂—OC(O)C(NH₂)—CH(CH₃)₂ group and a —CH₂—C(O)N(CH₃)CH₂CO₂R^{a} group;
R² represents a hydrogen atom, methyl group, fluorine atom, chlorine atom, bromine atom or a —CN group;
R³ represents a linear C₉₋₁₈ alkyl group,
   wherein the alkyl group is unsubstituted or substituted by one or more substituents selected from a fluorine atom, a linear or branched C₁₋₄ alkyl group, and a linear or branched C₁₋₃alkoxy group;
R⁴ is selected from the group consisting of a hydrogen atom and a linear or branched C₁₋₄ alkyl group;
R^{a} is selected from the group consisting of a hydrogen atom and a linear or branched C₁₋₄ alkyl group;
L represents a direct bond, —O—, —S— or a carbonyl group; characterized in that when R² represents a hydrogen atom, L represents a —O—.

In one embodiment, in the compound of formula (I),
R¹ is selected from the group consisting of a hydrogen atom, a linear or branched C₁₋₄ alkyl group, a —CH₂CF₃ group, a —(CH₂)₂₋₉—OH group, a —CH₂—CH(OH)—CH₂—OH, a —CH(CH₂OH)₂ group, a cyclohexyl group, a —(CH₂)₂-(2,5-dioxopyrrolidin-1-yl) group, a —(CH₂)₂-(2-oxopyrrolidin-1-yl) group, a —(CH₂)-(5-methyl-2-oxo-1,3-dioxol-4-yl) group, a —CH₂-phenyl group, a —(CH₂CH₂O)₂₋₃—R^{a} group, a —CH(CH₃)—OC(O)OCH(CH₃)₂ group, a —CH(CH₃)—OC(O)OC(CH₃)₃ group, a —CH(CH₃)—OC(O)O(CH₂)₈CH₃ group, a —CH(CH₃)—OC(O)O-cyclohexyl group, a —CH(CH₃)—OC(O)O—CH₂-phenyl group, a —CH(CH₃)—OC(O)O(CH₂CH₂O)₁₋₂—R^{a} group, a —CH(CH₃)—OC(O)O(CH₂)₃OH group, a —(CH₂)₂—OC(O)C(NH₂)—CH(CH₃)₂ group and a —CH₂—C(O)N(CH₃)CH₂CO₂R^{a} group;
R² represents a hydrogen atom, methyl group, fluorine atom, chlorine atom or bromine atom;
R³ represents a linear C₉₋₁₇ alkyl group,
   wherein the alkyl group is unsubstituted or substituted by one or more substituents selected from a fluorine atom, a linear or branched C₁₋₄ alkyl group, and a linear or branched C₁₋₃ alkoxy group;
R⁴ is selected from the group consisting of a hydrogen atom and a linear or branched C₁₋₄ alkyl group;
R^{a} is selected from the group consisting of a hydrogen atom and a linear or branched C₁₋₄ alkyl group;
L represents a direct bond, —O— or —S—; characterized in that when R² represents a hydrogen atom, L represents a —O—.

In a particular embodiment, it is preferred that,
R³ represents a linear C₉₋₁₇ alkyl group,
   wherein the alkyl group is unsubstituted or substituted by one or more substituents selected from a fluorine atom, a methyl group, and an ethoxy group;
R⁴ is selected from the group consisting of a hydrogen atom and a linear or branched C₁₋₄ alkyl group; preferably R⁴ is selected from the group consisting of a hydrogen atom, i-propyl group and n-butyl group.

In a particular embodiment, it is preferred that the compound of formula (I) is represented by Formula (Ia).

In a particular embodiment, it is preferred that the compound of formula (I) is represented by Formula (Ib).

Particular individual compounds of the invention include:
4-(Dodecyloxy)-1H-pyrrole-2-carboxylic acid
Ethyl 4-(dodecyloxy)-1H-pyrrole-2-carboxylate
2-(2,5-Dioxopyrrolidin-1-yl)ethyl 4-(dodecyloxy)-1H-pyrrole-2-carboxylate
2-(2-Oxopyrrolidin-1-yl)ethyl 4-(dodecyloxy)-1H-pyrrole-2-carboxylate
2,2,2-trifluoroethyl 4-(dodecyloxy)-1H-pyrrole-2-carboxylate
2-Hydroxyethyl 4-(dodecyloxy)-1H-pyrrole-2-carboxylate
2-(2-(2-(2-hydroxyethoxy)ethoxy)ethoxy)ethyl 4-(dodecyloxy)-1H-pyrrole-2-carboxylate
1-((isopropoxycarbonyl)oxy)ethyl 4-(dodecyloxy)-1H-pyrrole-2-carboxylate
2-((2-ethoxy-2-oxoethyl)(methyl)amino)-2-oxoethyl 4-(dodecyloxy)-1H-pyrrole-2-carboxylate
2-((L-valyl)oxy)ethyl 4-(dodecyloxy)-1H-pyrrole-2-carboxylate
(5-methyl-2-oxo-1,3-dioxol-4-yl)methyl 4-(dodecyloxy)-1H-pyrrole-2-carboxylate
4-Decyl-3-fluoro-1H-pyrrole-2-carboxylic acid
3-Fluoro-4-undecyl-1H-pyrrole-2-carboxylic acid
4-Dodecyl-3-fluoro-1H-pyrrole-2-carboxylic acid
2,2,2-trifluoroethyl 4-dodecyl-3-fluoro-1H-pyrrole-2-carboxylate
2-(2-ethoxyethoxy)ethyl 4-dodecyl-3-fluoro-1H-pyrrole-2-carboxylate
1-((Isopropoxycarbonyl)oxy)ethyl 4-dodecyl-3-fluoro-1H-pyrrole-2-carboxylate
1-(((2-Methoxyethoxy)carbonyl)oxy)ethyl 4-dodecyl-3-fluoro-1H-pyrrole-2-carboxylate
4-oxo-3,5,8,11-tetraoxatridecan-2-yl 4-dodecyl-3-fluoro-1H-pyrrole-2-carboxylate
Ethyl 3-fluoro-4-tridecyl-1H-pyrrole-2-carboxylate
3-Fluoro-4-tridecyl-1H-pyrrole-2-carboxylic acid
Methyl 3-fluoro-4-tridecyl-1H-pyrrole-2-carboxylate
Isopropyl 3-fluoro-4-tridecyl-1H-pyrrole-2-carboxylate
Tert-butyl 3-fluoro-4-tridecyl-1H-pyrrole-2-carboxylate
Cyclohexyl 3-fluoro-4-tridecyl-1H-pyrrole-2-carboxylate
Benzyl 3-fluoro-4-tridecyl-1H-pyrrole-2-carboxylate
2,2,2-Trifluoroethyl 3-fluoro-4-tridecyl-1H-pyrrole-2-carboxylate
2-(2,5-Dioxopyrrolidin-1-yl)ethyl 3-fluoro-4-tridecyl-1H-pyrrole-2-carboxylate
2-(2-Oxopyrrolidin-1-yl)ethyl 3-fluoro-4-tridecyl-1H-pyrrole-2-carboxylate
(5-Methyl-2-oxo-1,3-dioxol-4-yl)methyl 3-fluoro-4-tridecyl-1H-pyrrole-2-carboxylate
2-((2-Ethoxy-2-oxoethyl)(methyl)amino)-2-oxoethyl 3-fluoro-4-tridecyl-1H-pyrrole-2-carboxylate
2-Hydroxyethyl 3-fluoro-4-tridecyl-1H-pyrrole-2-carboxylate
3-Hydroxypropyl 3-fluoro-4-tridecyl-1H-pyrrole-2-carboxylate
4-Hydroxybutyl 3-fluoro-4-tridecyl-1H-pyrrole-2-carboxylate
5-Hydroxypentyl 3-fluoro-4-tridecyl-1H-pyrrole-2-carboxylate
6-Hydroxyhexyl 3-fluoro-4-tridecyl-1H-pyrrole-2-carboxylate
7-Hydroxyheptyl 3-fluoro-4-tridecyl-1H-pyrrole-2-carboxylate
8-Hydroxyoctyl 3-fluoro-4-tridecyl-1H-pyrrole-2-carboxylate
9-Hydroxynonyl 3-fluoro-4-tridecyl-1H-pyrrole-2-carboxylate
2,3-Dihydroxypropyl 3-fluoro-4-tridecyl-1H-pyrrole-2-carboxylate
1,3-Dihydroxypropan-2-yl 3-fluoro-4-tridecyl-1H-pyrrole-2-carboxylate
2-(2-(2-(2-Hydroxyethoxy)ethoxy)ethoxy)ethyl 3-fluoro-4-tridecyl-1H-pyrrole-2-carboxylate
2-(2-Ethoxyethoxy)ethyl 3-fluoro-4-tridecyl-1H-pyrrole-2-carboxylate
1-((Isopropoxycarbonyl)oxy)ethyl 3-fluoro-4-tridecyl-1H-pyrrole-2-carboxylate
1-((Tert-butoxycarbonyl)oxy)ethyl 3-fluoro-4-tridecyl-1H-pyrrole-2-carboxylate
1-(((Nonyloxy)carbonyl)oxy)ethyl 3-fluoro-4-tridecyl-1H-pyrrole-2-carboxylate
1-(((Cyclohexyloxy)carbonyl)oxy)ethyl 3-fluoro-4-tridecyl-1H-pyrrole-2-carboxylate
1-(((Benzyloxy)carbonyl)oxy)ethyl 3-fluoro-4-tridecyl-1H-pyrrole-2-carboxylate
1-(((2-Methoxyethoxy)carbonyl)oxy)ethyl 3-fluoro-4-tridecyl-1H-pyrrole-2-carboxylate
1-(((3-Hydroxypropoxy)carbonyl)oxy)ethyl 3-fluoro-4-tridecyl-1H-pyrrole-2-carboxylate
4-Oxo-3,5,8,11-tetraoxatridecan-2-yl 3-fluoro-4-tridecyl-1H-pyrrole-2-carboxylate
3-Fluoro-4-tetradecyl-1H-pyrrole-2-carboxylic acid
3-Fluoro-4-pentadecyl-1H-pyrrole-2-carboxylic acid
3-Fluoro-4-heptadecyl-1H-pyrrole-2-carboxylic acid
5-Dodecyl-3-fluoro-1H-pyrrole-2-carboxylic acid
3-Chloro-4-decyl-1H-pyrrole-2-carboxylic acid
3-Chloro-4-undecyl-1H-pyrrole-2-carboxylic acid
3-Chloro-4-dodecyl-1H-pyrrole-2-carboxylic acid
9-Hydroxynonyl 3-chloro-4-dodecyl-1H-pyrrole-2-carboxylate
2-(2,5-dioxopyrrolidin-1-yl)ethyl 3-chloro-4-dodecyl-1H-pyrrole-2-carboxylate
3-Chloro-4-tridecyl-1H-pyrrole-2-carboxylic acid
3-Chloro-4-pentadecyl-1H-pyrrole-2-carboxylic acid
3-Chloro-4-hexadecyl-1H-pyrrole-2-carboxylic acid
3-Chloro-5-undecyl-1H-pyrrole-2-carboxylic acid
3-Chloro-5-dodecyl-1H-pyrrole-2-carboxylic acid
3-Chloro-5-tridecyl-1H-pyrrole-2-carboxylic acid
3-chloro-5-tetradecyl-1H-pyrrole-2-carboxylic acid
3-Bromo-4-tridecyl-1H-pyrrole-2-carboxylic acid
1-Butyl-3-fluoro-4-tridecyl-1H-pyrrole-2-carboxylic acid
3-Fluoro-1-isopropyl-4-tridecyl-1H-pyrrole-2-carboxylic acid
4-(Decyloxy)-3-fluoro-1H-pyrrole-2-carboxylic acid
3-Fluoro-4-(undecyloxy)-1H-pyrrole-2-carboxylic acid
4-(Dodecyloxy)-3-fluoro-1H-pyrrole-2-carboxylic acid
3-Fluoro-4-(tridecyloxy)-1H-pyrrole-2-carboxylic acid
3-Fluoro-4-(tetradecyloxy)-1H-pyrrole-2-carboxylic acid
4-(Dodecylthio)-3-fluoro-1H-pyrrole-2-carboxylic acid
3-Chloro-4-(nonyloxy)-1H-pyrrole-2-carboxylic acid
3-Chloro-4-(decyloxy)-1H-pyrrole-2-carboxylic acid
3-chloro-4-(undecyloxy)-1H-pyrrole-2-carboxylic acid
3-Chloro-4-(dodecyloxy)-1H-pyrrole-2-carboxylic acid
2,2,2-trifluoroethyl 3-chloro-4-(dodecyloxy)-1H-pyrrole-2-carboxylate
9-hydroxynonyl 3-chloro-4-(dodecyloxy)-1H-pyrrole-2-carboxylate
2-(2-ethoxyethoxy)ethyl 3-chloro-4-(dodecyloxy)-1H-pyrrole-2-carboxylate
2,3-Dihydroxypropyl 3-chloro-4-(dodecyloxy)-1H-pyrrole-2-carboxylate
1-((isopropoxycarbonyl)oxy)ethyl 3-chloro-4-(dodecyloxy)-1H-pyrrole-2-carboxylate
4-Oxo-3,5,8,11-tetraoxatridecan-2-yl 3-chloro-4-(dodecyloxy)-1H-pyrrole-2-carboxylate
1-(((3-hydroxypropoxy)carbonyl)oxy)ethyl 3-chloro-4-(dodecyloxy)-1H-pyrrole-2-carboxylate
(5-methyl-2-oxo-1,3-dioxol-4-yl)methyl 3-chloro-4-(dodecyloxy)-1H-pyrrole-2-carboxylate
3-Chloro-4-(tridecyloxy)-1H-pyrrole-2-carboxylic acid
3-Chloro-4-(tetradecyloxy)-1H-pyrrole-2-carboxylic acid
3-Fluoro-4-pentadecanoyl-1H-pyrrole-2-carboxylic acid
4-(12-Ethoxydodecyl)-3-fluoro-1H-pyrrole-2-carboxylic acid
3-fluoro-4-(2-fluorotridecyl)-1H-pyrrole-2-carboxylic acid
4-(2,2-Difluorotridecyl)-3-fluoro-1H-pyrrole-2-carboxylic acid
4-(3,3-dimethyldodecyl)-3-fluoro-1H-pyrrole-2-carboxylic acid
4-((2,2-dimethyltridecyl)oxy)-3-fluoro-1H-pyrrole-2-carboxylic acid
4-((2,2-difluorotetradecyl)oxy)-3-fluoro-1H-pyrrole-2-carboxylic acid
4-((2,2-difluoroundecyl)oxy)-3-fluoro-1H-pyrrole-2-carboxylic acid
3-chloro-4-((2-fluorotetradecyl)oxy)-1H-pyrrole-2-carboxylic acid
3-chloro-4-((9-ethoxynonyl)oxy)-1H-pyrrole-2-carboxylic acid
3-Methyl-4-tridecyl-1H-pyrrole-2-carboxylic acid
4-(2,2-Dimethyldodecyl)-3-fluoro-1H-pyrrole-2-carboxylic acid
2,2,2-Trifluoroethyl 3-fluoro-4-(undecyloxy)-1H-pyrrole-2-carboxylate
2-(2-Ethoxyethoxy)ethyl 3-fluoro-4-(undecyloxy)-1H-pyrrole-2-carboxylate
1-((Isopropoxycarbonyl)oxy)ethyl 3-fluoro-4-(undecyloxy)-1H-pyrrole-2-carboxylate
1-(((2-Methoxyethoxy)carbonyl)oxy)ethyl 3-fluoro-4-(undecyloxy)-1H-pyrrole-2-carboxylate
4-Oxo-3,5,8,11-tetraoxatridecan-2-yl 3-fluoro-4-(undecyloxy)-1H-pyrrole-2-carboxylate
2,2,2-Trifluoroethyl 3-chloro-4-tridecyl-1H-pyrrole-2-carboxylate
2-(2-Ethoxyethoxy)ethyl 3-chloro-4-tridecyl-1H-pyrrole-2-carboxylate
1-((Isopropoxycarbonyl)oxy)ethyl 3-chloro-4-tridecyl-1H-pyrrole-2-carboxylate
1-(((2-methoxyethoxy)carbonyl)oxy)ethyl 3-chloro-4-tridecyl-1H-pyrrole-2-carboxylate
4-Oxo-3,5,8,11-tetraoxatridecan-2-yl 3-chloro-4-tridecyl-1H-pyrrole-2-carboxylate
2,2,2-trifluoroethyl 3-chloro-5-dodecyl-1H-pyrrole-2-carboxylate
2-(2-ethoxyethoxy)ethyl 3-chloro-5-dodecyl-1H-pyrrole-2-carboxylate
1-((isopropoxycarbonyl)oxy)ethyl 3-chloro-5-dodecyl-1H-pyrrole-2-carboxylate
1-(((2-methoxyethoxy)carbonyl)oxy)ethyl 3-chloro-5-dodecyl-1H-pyrrole-2-carboxylate
4-oxo-3,5,8,11-tetraoxatridecan-2-yl 3-chloro-5-dodecyl-1H-pyrrole-2-carboxylate
2,3-dihydroxypropyl 3-chloro-5-dodecyl-1H-pyrrole-2-carboxylate
3-Fluoro-5-undecyl-1H-pyrrole-2-carboxylic acid
3-Fluoro-5-tridecyl-1H-pyrrole-2-carboxylic acid
3-Fluoro-5-tetradecyl-1H-pyrrole-2-carboxylic acid
3-Fluoro-5-pentadecyl-1H-pyrrole-2-carboxylic acid
3-Fluoro-5-hexadecyl-1H-pyrrole-2-carboxylic acid
3-Fluoro-5-heptadecyl-1H-pyrrole-2-carboxylic acid
3-Fluoro-5-octadecyl-1H-pyrrole-2-carboxylic acid
3-Fluoro-5-octadecyl-1H-pyrrole-2-carboxylic acid
3-Chloro-5-(2,2-dimethyldodecyl)-1H-pyrrole-2-carboxylic acid
3-Chloro-5-(3,3-difluorododecyl)-1H-pyrrole-2-carboxylic acid
3-Cyano-5-dodecyl-1H-pyrrole-2-carboxylic acid
3-Chloro-5-dodecyl-1-methyl-1H-pyrrole-2-carboxylic acid
3-Fluoro-5-(14-fluorotetradecyl)-1H-pyrrole-2-carboxylic acid
3-Fluoro-4-hexadecyl-1H-pyrrole-2-carboxylic acid
   or a pharmaceutically acceptable salt, or a solvate, or a N-oxide, or a tautomer, or a stereoisomer, or an isotopically-labelled derivative thereof.

Of particular interest are the compounds:
4-Decyl-3-fluoro-1H-pyrrole-2-carboxylic acid
3-Fluoro-4-undecyl-1H-pyrrole-2-carboxylic acid
4-Dodecyl-3-fluoro-1H-pyrrole-2-carboxylic acid
2,2,2-trifluoroethyl 4-dodecyl-3-fluoro-1H-pyrrole-2-carboxylate
2-(2-ethoxyethoxy)ethyl 4-dodecyl-3-fluoro-1H-pyrrole-2-carboxylate
1-((Isopropoxycarbonyl)oxy)ethyl 4-dodecyl-3-fluoro-1H-pyrrole-2-carboxylate
4-oxo-3,5,8,11-tetraoxatridecan-2-yl 4-dodecyl-3-fluoro-1H-pyrrole-2-carboxylate
3-Fluoro-4-tridecyl-1H-pyrrole-2-carboxylic acid
2,2,2-Trifluoroethyl 3-fluoro-4-tridecyl-1H-pyrrole-2-carboxylate
2-(2,5-Dioxopyrrolidin-1-yl)ethyl 3-fluoro-4-tridecyl-1H-pyrrole-2-carboxylate
(5-Methyl-2-oxo-1,3-dioxol-4-yl)methyl 3-fluoro-4-tridecyl-1H-pyrrole-2-carboxylate
2-((2-Ethoxy-2-oxoethyl)(methyl)amino)-2-oxoethyl 3-fluoro-4-tridecyl-1H-pyrrole-2-carboxylate
9-Hydroxynonyl 3-fluoro-4-tridecyl-1H-pyrrole-2-carboxylate
2,3-Dihydroxypropyl 3-fluoro-4-tridecyl-1H-pyrrole-2-carboxylate
1,3-Dihydroxypropan-2-yl 3-fluoro-4-tridecyl-1H-pyrrole-2-carboxylate
2-(2-Ethoxyethoxy)ethyl 3-fluoro-4-tridecyl-1H-pyrrole-2-carboxylate
1-((Isopropoxycarbonyl)oxy)ethyl 3-fluoro-4-tridecyl-1H-pyrrole-2-carboxylate
1-((Tert-butoxycarbonyl)oxy)ethyl 3-fluoro-4-tridecyl-1H-pyrrole-2-carboxylate
1-(((Cyclohexyloxy)carbonyl)oxy)ethyl 3-fluoro-4-tridecyl-1H-pyrrole-2-carboxylate
1-(((Benzyloxy)carbonyl)oxy)ethyl 3-fluoro-4-tridecyl-1H-pyrrole-2-carboxylate
1-(((2-Methoxyethoxy)carbonyl)oxy)ethyl 3-fluoro-4-tridecyl-1H-pyrrole-2-carboxylate
1-(((3-Hydroxypropoxy)carbonyl)oxy)ethyl 3-fluoro-4-tridecyl-1H-pyrrole-2-carboxylate
4-Oxo-3,5,8,11-tetraoxatridecan-2-yl 3-fluoro-4-tridecyl-1H-pyrrole-2-carboxylate
3-Fluoro-4-tetradecyl-1H-pyrrole-2-carboxylic acid
3-Fluoro-4-pentadecyl-1H-pyrrole-2-carboxylic acid
3-Fluoro-4-heptadecyl-1H-pyrrole-2-carboxylic acid
5-Dodecyl-3-fluoro-1H-pyrrole-2-carboxylic acid
3-Chloro-4-undecyl-1H-pyrrole-2-carboxylic acid
3-Chloro-4-dodecyl-1H-pyrrole-2-carboxylic acid
3-Chloro-4-tridecyl-1H-pyrrole-2-carboxylic acid
3-Chloro-5-undecyl-1H-pyrrole-2-carboxylic acid
3-Chloro-5-dodecyl-1H-pyrrole-2-carboxylic acid
3-Chloro-5-tridecyl-1H-pyrrole-2-carboxylic acid
4-(Decyloxy)-3-fluoro-1H-pyrrole-2-carboxylic acid
3-Fluoro-4-(undecyloxy)-1H-pyrrole-2-carboxylic acid
4-(Dodecyloxy)-3-fluoro-1H-pyrrole-2-carboxylic acid
3-Fluoro-4-(tridecyloxy)-1H-pyrrole-2-carboxylic acid
3-Fluoro-4-(tetradecyloxy)-1H-pyrrole-2-carboxylic acid
3-Chloro-4-(decyloxy)-1H-pyrrole-2-carboxylic acid
3-chloro-4-(undecyloxy)-1H-pyrrole-2-carboxylic acid
3-Chloro-4-(dodecyloxy)-1H-pyrrole-2-carboxylic acid
1-((isopropoxycarbonyl)oxy)ethyl 3-chloro-4-(dodecyloxy)-1H-pyrrole-2-carboxylate
4-Oxo-3,5,8,11-tetraoxatridecan-2-yl 3-chloro-4-(dodecyloxy)-1H-pyrrole-2-carboxylate
3-Chloro-4-(tridecyloxy)-1H-pyrrole-2-carboxylic acid
3-Chloro-4-(tetradecyloxy)-1H-pyrrole-2-carboxylic acid
4-(12-Ethoxydodecyl)-3-fluoro-1H-pyrrole-2-carboxylic acid
3-fluoro-4-(2-fluorotridecyl)-1H-pyrrole-2-carboxylic acid
4-(3,3-dimethyldodecyl)-3-fluoro-1H-pyrrole-2-carboxylic acid
4-((2,2-dimethyltridecyl)oxy)-3-fluoro-1H-pyrrole-2-carboxylic acid
4-((2,2-difluorotetradecyl)oxy)-3-fluoro-1H-pyrrole-2-carboxylic acid
4-(2,2-Dimethyldodecyl)-3-fluoro-1H-pyrrole-2-carboxylic acid
2,2,2-Trifluoroethyl 3-fluoro-4-(undecyloxy)-1H-pyrrole-2-carboxylate
1-((Isopropoxycarbonyl)oxy)ethyl 3-fluoro-4-(undecyloxy)-1H-pyrrole-2-carboxylate
1-(((2-Methoxyethoxy)carbonyl)oxy)ethyl 3-fluoro-4-(undecyloxy)-1H-pyrrole-2-carboxylate
4-Oxo-3,5,8,11-tetraoxatridecan-2-yl 3-fluoro-4-(undecyloxy)-1H-pyrrole-2-carboxylate
1-(((2-methoxyethoxy)carbonyl)oxy)ethyl 3-chloro-5-dodecyl-1H-pyrrole-2-carboxylate
4-oxo-3,5,8,11-tetraoxatridecan-2-yl 3-chloro-5-dodecyl-1H-pyrrole-2-carboxylate
3-Fluoro-5-undecyl-1H-pyrrole-2-carboxylic acid
3-Fluoro-5-tridecyl-1H-pyrrole-2-carboxylic acid
3-Fluoro-5-tetradecyl-1H-pyrrole-2-carboxylic acid
3-Fluoro-5-pentadecyl-1H-pyrrole-2-carboxylic acid
3-Fluoro-5-hexadecyl-1H-pyrrole-2-carboxylic acid
3-Fluoro-5-heptadecyl-1H-pyrrole-2-carboxylic acid
3-Fluoro-5-octadecyl-1H-pyrrole-2-carboxylic acid
3-Fluoro-5-octadecyl-1H-pyrrole-2-carboxylic acid
3-Fluoro-4-hexadecyl-1H-pyrrole-2-carboxylic acid
   or a pharmaceutically acceptable salt, or a solvate, or a N-oxide, or a tautomer, or a stereoisomer, or an isotopically-labelled derivative thereof.

General Synthetic Procedures

The compounds of the invention can be prepared using the methods and procedures described herein, or using similar methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Starting compounds are commercially available or may be obtained following the conventional synthetic methods already known in the art.

Additionally, as will be apparent to those skilled in the art, conventional protecting groups may be necessary to prevent certain functional groups from undergoing undesired reactions. The choice of a suitable protecting group for a particular functional group, as well as suitable conditions for protection and deprotection, are well known in the art. For example, numerous protecting groups, and their introduction and removal are described in T. W. Greene and G. M. Wuts, Protecting Groups in Organic Synthesis, Third Edition, Wiley, New York, 1999, and references cited therein.

Processes for preparing compounds of the invention are provided as further embodiments of the invention and are illustrated by the procedures below.

Specific synthetic processes not covered by Schemes 1-8 are described in detail in the experimental section.

According to one embodiment of the present invention, compounds of general formula (I′) and (I″), subsets of general formula (I), wherein R¹-R⁴ and L are as defined in the claims, may be prepared by the following synthetic route as illustrated in Scheme 1:

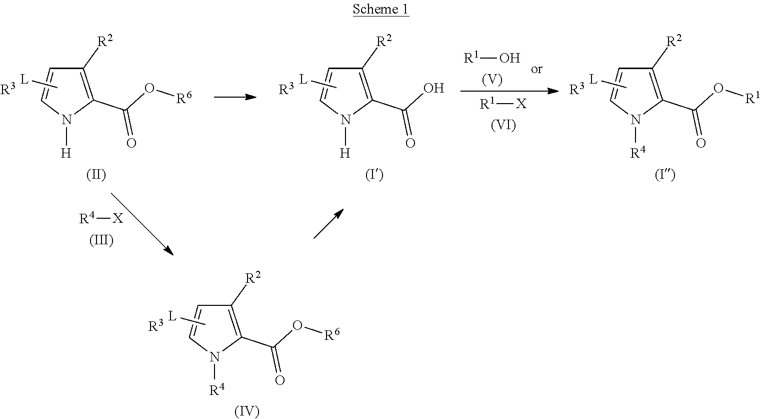

Compounds of general formula (I″), a subset of general formula (I), wherein R¹ is other than a hydrogen atom, may be obtained from compounds of general formula (I′), a subset of general formula (I), wherein R¹ is a hydrogen atom, by reaction with alcohols of formula (V) in the presence of a base such as 4-dimethylaminopyridine or triethylamine and a coupling reagent such as 3-((ethylimino) methyleneamino)-N,N-dimethylpropan-1-aminium chloride (EDCl-HCl) or dicyclohexylcarbodiimide (DCC), in a solvent such as methylene chloride at room temperature. Compounds of formula (I″) may also be prepared from compounds of formula (I′) following a different synthetic approach. Reaction of compounds of formula (I′) with a suitable chlorinating reagent such as oxalyl chloride in the presence of a catalytic amount of N,N-dimethylformamide in a solvent such as methylene chloride at room temperature gives rise to intermediate acid chlorides which may be treated with alcohols of general formula (V) without the presence of a base or in the presence of a base such as triethylamine, without the use of a solvent or in a solvent such as methylene chloride at temperatures ranging from 0° C. to room temperature to provide compounds of formula (I″). Alternatively, compounds of formula (I″) may also be obtained by reaction of compounds of formula (I′) with haloderivatives of formula (VI), wherein X represents a halogen atom, in the presence of a base such as potassium carbonate or triethylamine, in a solvent such as acetonitrile or N,N-dimethylformamide at temperatures ranging from room temperature to reflux.

In a particular case, compounds of formula (I″), in which the residue at R¹ contains an alcohol or diol moiety functionalized with an appropriate protecting group such as benzyl (Bn) or benzylidene acetal, may be deprotected at the alcohol or diol moiety under standard conditions (Greene's Protective Groups in Organic Synthesis, ISBN; 0471697540).

In another particular case, compounds of formula (I″), in which the residue at R¹ contains an amine moiety functionalized with an appropriate protecting group such as tert-butoxycarbonyl (BOC), may be deprotected at the amine moiety under standard conditions (Greene's Protective Groups in Organic Synthesis, ISBN; 0471697540).

Compounds of formula (I′), a subset of formula (I), wherein R¹ is a hydrogen atom, may be obtained from compounds of formulas (II) and (IV). Compounds of formulas (II) and (IV), wherein R⁶ represents an alkyl group such as methyl or ethyl group, may be treated with a suitable base such as lithium hydroxide, sodium hydroxide or potassium hydroxide, in a solvent such as methanol, ethanol or tetrahydrofuran, with or without the presence of water as co-solvent, at temperatures ranging from ambient temperature to reflux, to furnish compounds of formula (I′). Esters of formula (IV), wherein R⁴ is a C₁₋₄alkyl group, may be prepared from compounds of formula (II) by treatment with a suitable base such as sodium hydride in a solvent such as N,N-dimethylformamide, followed by addition of an haloderivative of formula (III), wherein X represents a halogen atom, such as 1-iodobutane or 2-iodopropane, at temperatures ranging from 0° C. to room temperature.

In a particular case, compounds of general formulas (IIa) and (IIb), a subset of general formula (II), wherein L is a direct bond, R⁷ represents a linear or branched C₈₋₁₉ alkyl group which may be substituted by one or more halogen atoms and R² is as defined in the claims, may be prepared by the following synthetic route as illustrated in Scheme 2:

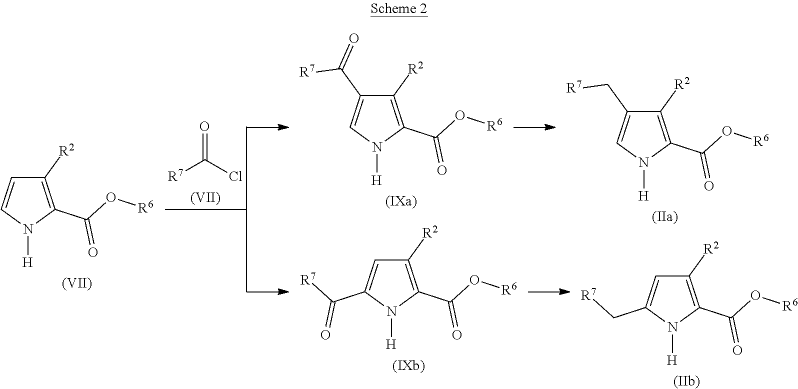

Pyrroles of formula (VII) may be reacted with acid chlorides of formula (VIII) in the presence of a Lewis acid such as zinc(II) chloride, aluminium(III) chloride, tin(IV) chloride or boron trifluoride diethyl etherate, in a solvent such as methylene chloride, 1,2-dichloroethane or benzene, at temperatures ranging from 0° C. to room temperature, to furnish ketones of formulas (IXa) and (IXb). The ratio among regioisomers (IXa) and (IXb) may vary depending on the Lewis acid and the reaction conditions used. Reduction of ketones of formulas (IXa) and (IXb) by treatment with triethylsilane and trifluoroacetic acid, with or without the use of a Lewis acid such as boron trifluoride diethyl etherate, at room temperature, furnishes compounds of formulas (IIa) and (IIb) respectively.

In another particular case, compounds of formula (IIb), wherein R² is a fluorine or chlorine atom or a cyano group, may also be obtained as illustrated in Scheme 3:

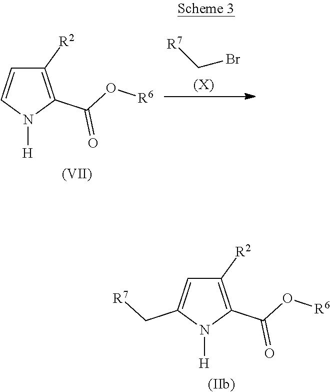

Reaction of pyrroles of formula (VII) with bromoderivatives of formula (X) in the presence of norbornene, a palladium catalyst such as dichlorobis(acetonitrile)palladium (II) and a base such as potassium hydrogenphosphate, in a solvent such as N,N-dimethylacetamide, at temperatures ranging from 60° C. to reflux, furnishes compounds of formula (IIb).

Alternatively, compounds of formula (IIb), wherein R² is a chlorine atom, may also be prepared by an alternative synthetic route as illustrated in Scheme 4:

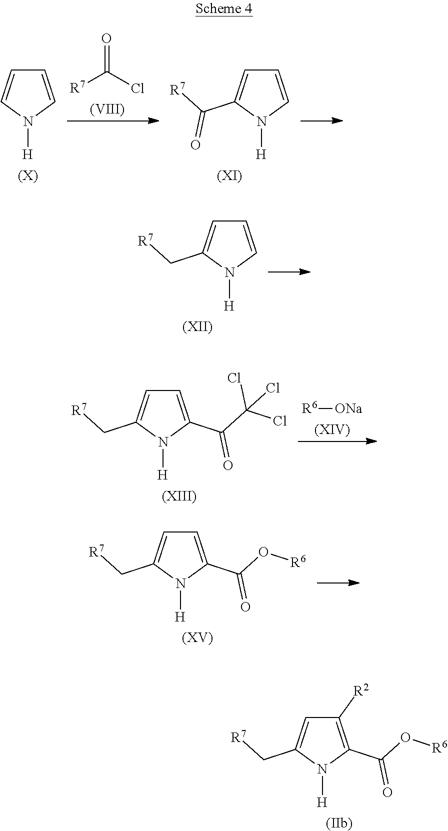

Reaction of pyrrole (XI) with acid chlorides of formula (VIII) in the presence of zinc in a solvent such as toluene at room temperature provides ketones of formula (XII). Treatment of molecules of formula (XII) with hydrazine hydrate in the presence of a base such as potassium hydroxide in a solvent such as diethylenglycol at 200° C. gives rise to compounds of formula (XIII), which may be converted into trichloroketones of formula (XIV) by reaction with 2,2,2-trichloroacetyl chloride in the presence of a base such as 2,6-lutidine, in a solvent such as 1,4-dioxane at 85° C. Reaction of trichloroketones of formula (XIV) with sodium alcoxides of formula (XV) such as sodium methoxide or sodium ethoxide in a solvent such as methanol or ethanol at room temperature, furnishes esters of formula (XVI) which may be converted into compounds of formula (IIb), wherein R² is a chlorine atom, by reaction with a chlorinating agent, such as N-chlorosuccinimide, in a solvent such as chloroform at 40° C.

In a particular case, compounds of general formula (IIe), wherein L is an oxygen atom and R² and R³ are as defined in the claims, may be prepared by the following synthetic route as illustrated in Scheme 5:

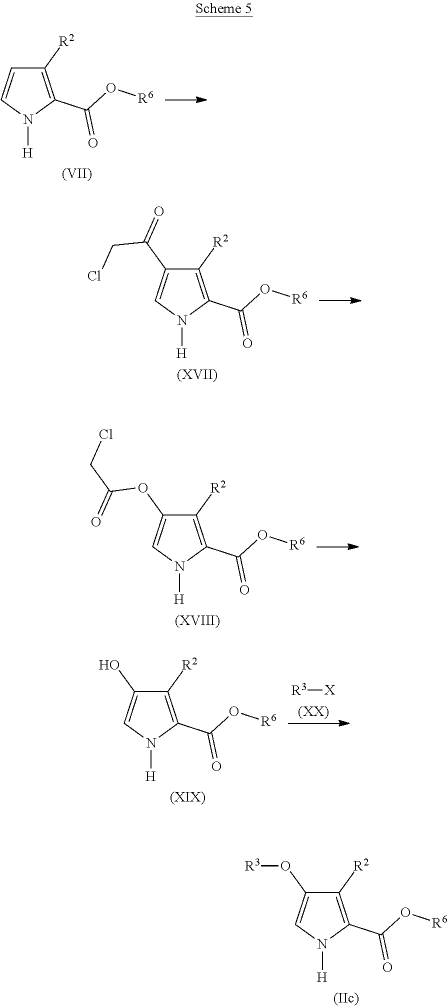

Reaction of pyrroles of formula (VII) with 2-chloroacetyl chloride in the presence of a Lewis acid such as aluminium(III) chloride, in a solvent such as methylene chloride at room temperature, provides chloroketones of formula (XVII) which may be converted into 2-chloroacetyl esters of formula (XVIII) by treatment with 3-chloroperbenzoic acid in the presence of sodium hydrogen carbonate, in a solvent such as methylene chloride at room temperature. Treatment of esters of formula (XVIII) with a suitable base such as potassium carbonate in a mixture of methanol and water as solvents at room temperature, furnishes compounds of formula (XIX). Selective O-alkylation of compounds of formula (XIX) may be achieved by reaction with haloderivatives of formula (XX), wherein X is a halogen atom, in the presence of a base such as potassium carbonate in a solvent such as N,N-dimethylformamide at 100° C. to yield compounds of general formula (IIe).

In another particular case, compounds of formula (IId), wherein L is a sulphur atom and R² and R³ are as defined in the claims, may be prepared by the following synthetic route as illustrated in Scheme 6:

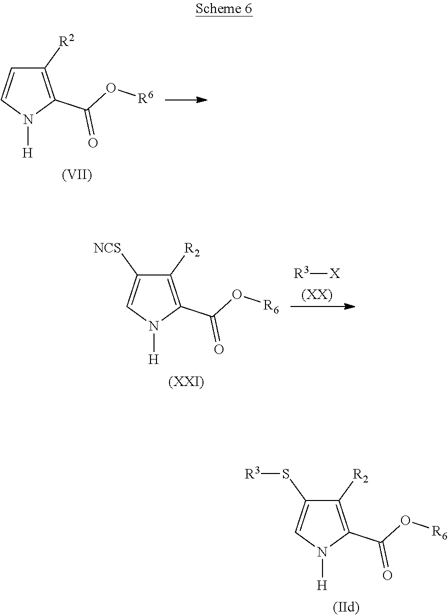

Pyrroles of formula (VII) may be reacted with a mixture of potassium thiocyanate and bromine in a solvent such as methanol at temperatures ranging from −78° C. to room temperature, to give rise to thiocyanates of formula (XXI). Thioethers of formula (IId) may be prepared from thiocyanates of formula (XXI) by reaction with haloderivatives of formula (XX), wherein X is a halogen atom, in the presence of a base such as sodium hydroxide in a mixture of tert-butanol and water as solvents at 60° C.

In yet another particular case, compounds of formulas (IIe) and (IIf), wherein L is a direct bond and R⁸ represents a linear or branched C₇₋₁₈ alkyl group and R² is as defined in the claims, may be prepared by the following synthetic route as illustrated in Scheme 7:

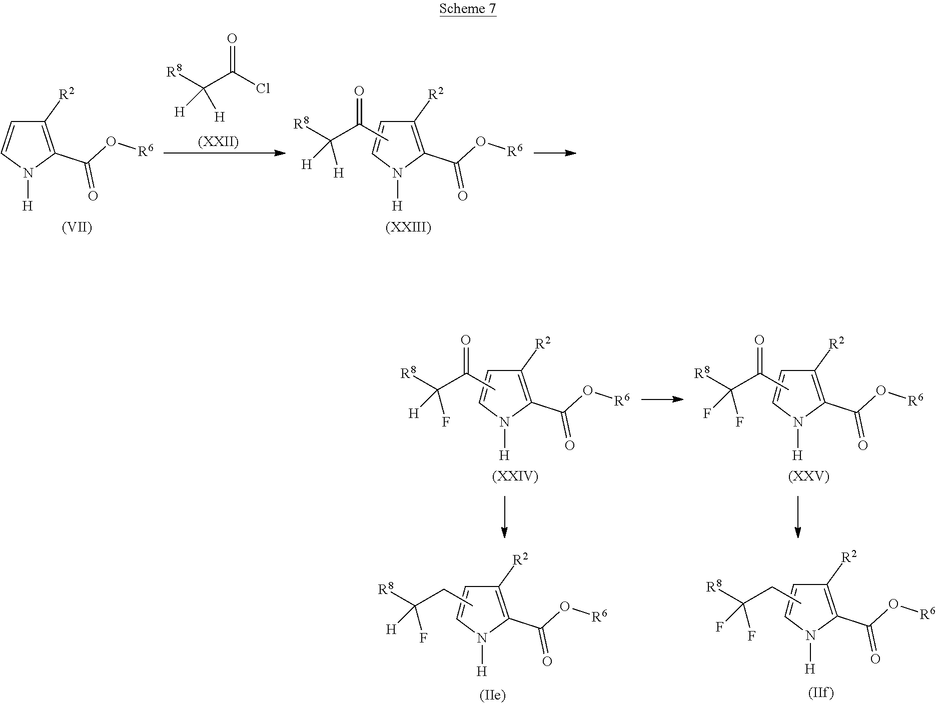

Pyrroles of formula (VII) may be reacted with acid chlorides of formula (XXII) in the presence of a Lewis acid such as aluminium(III) chloride, in a solvent such as methylene chloride at temperatures ranging from 0° C. to room temperature to yield ketones of formula (XXIII). Treatment of ketones of formula (XXIII) with a suitable base such as lithium diisopropylamide (LDA) in a solvent such as tetrahydrofuran followed by the addition of N-fluorobenzenesulfonimide at temperatures ranging from −78° C. to room temperature, gives rise to fluorocompounds of formula (XXIV). Reagents and reaction conditions used in the previous synthetic step may be also used to convert fluorocompounds of formula (XXIV) into difluoroderivatives of formula (XXV). Reaction of ketones of formulas (XXIV) and (XXV) with triethylsilane and trifluoroacetic acid at room temperature provides compounds of formulas (IIe) and (IIf).

In yet another particular case, compounds of formula (IIg), wherein R⁹ and R¹⁰ represents a linear or branched C₁₋₆ alkyl group and R² is as defined in the claims, may be prepared by the following synthetic route as illustrated in Scheme 8:

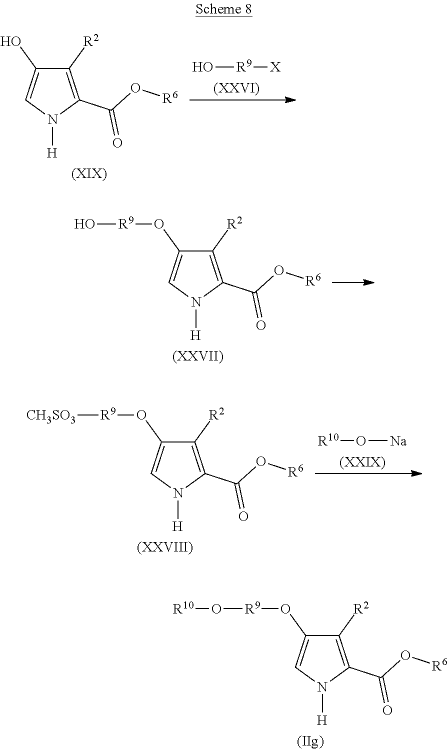

Selective O-alkylation of compounds of formula (XIX) may be achieved by reaction with haloalcohols of formula (XXVI), wherein X is a halogen atom, in the presence of a base such as potassium carbonate in a solvent such as N,N-dimethylformamide at 100° C. to yield compounds of general formula (XXVII). Alcohols of formula (XXVII) may be converted into methansulfonates of formula (XXVIII) by reaction with methanesulfonyl chloride in a solvent such as pyridine at 0° C. Methansulfonates of formula (XXVIII) may be reacted with sodium alcoxides of formula (XXIX) in a solvent such as methanol or ethanol at temperatures ranging from 0° C. to reflux to furnish compounds of formula (IIg).

### EXAMPLES

The syntheses of the compounds of the invention are illustrated by the following Examples (1 to 132) including Intermediates (1 to 64) which do not limit the scope of the invention in any way.

General

Reagents, starting materials, and solvents were purchased from commercial suppliers and used as received. Commercial intermediates are referred to in the experimental section by their IUPAC name. Ether refers to diethyl ether, unless otherwise specified. Concentration or evaporation refer to evaporation under vacuum using a Büchi rotatory evaporator. Reaction products were purified, when necessary, by flash chromatography on silica gel (40-63 μm) with the solvent system indicated. Purifications in reverse phase were made in a Biotage Isolera® automated purification system equipped with a C18 column and using a gradient, unless otherwise stated, of water-acetonitrile/MeOH (1:1) (0.1% v/v ammonium formate both phases) from 0% to 100% acetonitrile/MeOH (1:1) in 40 column volumes. The conditions “formic acid buffer” refer to the use of 0.1% v/v formic acid in both phases. The appropriate fractions were collected and the solvents evaporated under reduced pressure and/or lyophilized. Purifications in reverse phase were also made in a Biotage SP1® automated purification system equipped with a C18 column and using a gradient of, unless otherwise stated, water-acetonitrile/MeOH (1:1) (0.1% v/v ammonium formate both phases) from 0% to 100% acetonitrile/MeOH (1:1) in 80 column volumes. The conditions “formic acid buffer” refer to the use of 0.1% v/v formic acid in both phases. The appropriate fractions were collected and freeze dried.

Gas chromatography was performed using a Thermo Trace Ultra gas chromatograph, coupled to a DSQ mass detector. Injections were performed on a split/splitless injector and a HP-1 MS was the capillary column. Mass spectra were obtained by electron impact ionisation at 70 eV. Preparative HPLC-MS were performed on a Waters instrument equipped with a 2767 injector/collector, a 2525 binary gradient pump, a 2996 PDA detector, a 515 pump as a make-up pump and a ZQ4000 Mass spectrometer detector or on a Agilent 1200 Series coupled to an Agilent 6120 Mass spectrometer detector. Both systems were equipped with a Symmetry Prep C18 (19×300 mm, 7 μm) column or a XBridge Prep C18 (19×100 mm, 5 μm) column. The mobile phase was formic acid (0.4 mL), ammonia (0.1 mL), methanol (500 mL) and acetonitrile (500 mL) (B) and formic acid (0.5 mL), ammonia (0.125 mL) and water (1000 mL) (A), the specific gradients used are specified in each particular case. The flow rate was 20 ml/min. The UPLC chromatographic separations were obtained using a Waters Acquity UPLC system coupled to a SQD mass spectrometer detector. The system was equipped with an ACQUITY UPLC BEH C-18 (2.1×50 mm, 1.7 mm) column. The mobile phase was formic acid (0.4 ml), ammonia (0.1 ml), methanol (500 ml) and acetonitrile (500 ml) (B) and formic acid (0.5 ml), ammonia (0.125 ml) and water (1000 ml) (A). A gradient between 0 to 95% of B was used. The run time was 3 or 6 minutes. The injection volume was 0.5 microliter. Chromatograms were processed at 210 nM or 254 nM. Mass spectra of the chromatograms were acquired using positive and negative electrospray ionization.

1H Nuclear Magnetic Resonance Spectra were recorded on a Varian Mercury plus operating at a frequency of 400 MHz or a Varian VNMRS operating at 600 MHz and equipped with a cold probe for the 1H spectra. Samples were dissolved in the specified deuterated solvent.

Tetramethylsilane was used as reference.

Standard synthetic methods are described the first time they are used. Compounds synthesized with similar methods are referred to only by their starting materials, without full experimental detail. Slight modifications to the general experimental methods used are permitted in these cases. Specific synthetic transformations already described in the literature are referred to only by their bibliographical reference. Other specific methods are also described in full.

### Abbreviations

ACN Acetonitrile
br Broad
CDCl₃ Deuterated chloroform
CD₃OD Deuterated methanol
Celite® Diatomaceous earth
d Doublet
DCC Dicyclohexylcarbodiimide
DCE 1,2-Dichloroethane
DCM Dichloromethane, methylene chloride
dd Doublet of doublets
DIEA Diisopropylethyamine
DMAP Dimethylaminopyridine
DMF N,N-Dimethylformamide
DMSO Dimethylsulfoxide
DMSO-d₆ Deuterated Dimethylsulfoxide
EDC.HCl 3-((Ethylimino)methyleneamino)-N,N-dimethylpropan-1-aminium chloride
EtOAc Ethyl acetate
h Hour
hept Heptuplet
HPLC High-performance liquid chromatography
m Multiplet
mCPBA 3-chloroperbenzoic acid
min Minutes
MS Mass spectrometry
NCS N-Chlorosuccinimide
NMR Nuclear magnetic resonance
q Quartet
s Singlet
t Triplet
td Triple doublet
TEA Triethylamine
TFA Trifluoroacetic acid
THF Tetrahydrofuran

### Intermediate 1

### Methyl 4-(dodecyloxy)-1H-pyrrole-2-carboxylate

### a) Methyl 4-(2-chloroacetyl)-1H-pyrrole-2-carboxylate

To a cooled (0° C.) solution of aluminium(III) chloride (2398 mg, 17.98 mmol) in DCM (20 mL) was added dropwise 2-chloroacetyl chloride (1.49 mL, 18.78 mmol) and a solution of methyl 1H-pyrrole-2-carboxylate (500 mg, 4.0 mmol) in DCM (5 mL) and the resulting mixture was stirred at room temperature for 1 h. After cooling to 0° C. brine was added, the organic layer was separated and the aqueous layer was extracted with DCM (×3). The combined organic phases were washed with water and saturated sodium hydrogen carbonate solution, dried over magnesium sulfate, filtered and the solvent was evaporated to dryness to yield the title compound (681 mg, 85%).

MS (m/z): 202, 204 [M+1, M+3]⁺

¹H NMR δ (400 MHz, DMSO-d₆): 3.85 (s, 3H), 4.92 (s, 2H), 7.17-7.32 (m, 1H), 7.80-7.95 (m, 1H).

### b) Methyl 4-(2-chloroacetoxy)-1H-pyrrole-2-carboxylate

To a suspension of methyl 4-(2-chloroacetyl)-1H-pyrrole-2-carboxylate (Intermediate 1a, 681 mg, 3.38 mmol) in DCM (10 mL) were added sodium hydrogen phosphate (1966 mg, 13.85 mmol) and mCPBA (1915 mg, 7.77 mmol) and the resulting mixture was stirred at room temperature for 3 h. Water was added and the organic phase was separated, washed with water and saturated sodium hydrogen carbonate solution and dried over magnesium sulfate. The solvent was evaporated to dryness and the resulting residue was purified by flash chromatography (hexanes/EtOAc) to yield the title compound (441 mg, 59%).

MS (m/z): 218,220 [M+1, M+3]⁺

¹H NMR δ (400 MHz, CDCl₃): 3.86 (s, 3H), 4.25 (s, 2H), 6.77 (dd, J=2 and 1 Hz, 1H), 7.10 (dd, J=3 and 1 Hz, 1H).

### c) Methyl 4-hydroxy-1H-pyrrole-2-carboxylate

To a solution of methyl 4-(2-chloroacetoxy)-1H-pyrrole-2-carboxylate (Intermediate 1b, 441 mg, 2.02 mmol) in methanol (7 mL) were added water (1 mL) and potassium carbonate (420 mg, 3.04 mmol) and the resulting mixture was stirred at room temperature for 5 minutes. The organic solvent was evaporated, additional water was added and pH was adjusted to 5-6 by addition of 1M hydrochloric acid solution. The aqueous phase was extracted with EtOAc (×3) and the combined organic layers were washed with water, dried over magnesium sulfate, filtered and the solvent was evaporated to dryness. The crude was purified by flash chromatography (hexanes/EtOAc) to yield the title compound (233 mg, 81%).

MS (m/z): 142 [M+1]⁺

¹H NMR δ (400 MHz, CDCl₃): 3.83 (s, 4H), 6.50 (dd, J=2 and 1 Hz, 1H), 6.59 (dd, J=3 and 1 Hz, 1H).

### d) Methyl 4-(dodecyloxy)-1H-pyrrole-2-carboxylate

To a solution of methyl 4-hydroxy-1H-pyrrole-2-carboxylate (Intermediate 1c, 1950 mg, 13.82 mmol) in DMF (30 mL) were added potassium carbonate (3820 mg, 27.6 mmol) and 1-bromododecane (3.38 mL, 13.82 mmol) and the resulting mixture was heated at 100° C. for 20 h. After cooling to room temperature, the mixture was neutralized by addition of 1M hydrochloric acid solution before being partitioned between water and EtOAc. The organic layer was separated and the aqueous layer was washed with EtOAc (×2). The combined organic phases were washed with water and brine, dried over magnesium sulfate, filtered and the solvent was evaporated to dryness. The resulting crude was purified by flash chromatography (hexanes/EtOAc) and reverse phase chromatography (water/ACN both with 0.5% of formic acid) to yield the title compound (1450 mg, 34%).

MS (m/z): 310 [M+1]⁺

¹H NMR δ (400 MHz, CDCl₃): 0.74-1.01 (m, 3H), 1.15-1.51 (m, 18H), 1.66-1.80 (m, 2H), 3.83 (s, 3H), 3.86 (t, J=6 Hz, 2H), 6.43-6.61 (m, 2H).

### Intermediate 2

### Ethyl N-(2-chloroacetyl)-N-methylglycinate

To a cooled (0° C.) solution of ethyl methylglycinate (2.0 g, 17.07 mmol) and TEA (9.52 mL, 68.2 mmol) in DCM (40 mL) was carefully added chloroacetyl chloride (1.63 mL, 20.49 mmol) and the resulting mixture was stirred at room temperature for 1 h 30 minutes. The reaction mixture was then partitioned between 1M hydrochloric acid solution and DCM. The aqueous layer was separated and washed with DCM (×4). The combined organic phases were dried over magnesium sulfate, filtered and the solvent was evaporated to dryness to yield the title compound (2490 mg, 60%) which was used in the next synthetic step without any further purification.

MS (m/z): 194,196 [M+1/M+3]⁺

¹H NMR δ (400 MHz, CDCl₃): 1.28 (t, J=7 Hz, 2H), 3.16 (s, 3H), 4.13 (s, 2H), 4.14 (s, 2H), 4.20 (q, J=7 Hz, 2H).

### Intermediate 3

### Ethyl 4-decyl-3-fluoro-1H-pyrrole-2-carboxylate

### a) Decanoyl Chloride

To a cooled (0° C.) solution of decanoic acid (275 mg, 1.59 mmol) in DCM (8 mL) was added dropwise oxalyl chloride (0.56 mL, 6.39 mmol) and DMF (3 drops) and the mixture was stirred at room temperature for 4 h. The solvent was removed under reduced pressure to yield the title compound as a yellow oil (315 mg, 100%) which was used in the next synthetic step without further purification.

### b) Ethyl 4-decanoyl-3-fluoro-1H-pyrrole-2-carboxylate

To a solution of decanoyl chloride (Intermediate 3a, 291 mg, 1.53 mmol) in DCM (4 mL) under argon atmosphere at 0° C. was added portionwise aluminium(III) chloride (373 mg, 2.8 mmol) followed by a solution of ethyl 3-fluoro-1H-pyrrole-2-carboxylate (200 mg, 1.27 mmol) in DCM (4 mL) and the mixture was stirred at room temperature for three days. After cooling to 0° C., 1N hydrochloric acid solution (1 mL) was added dropwise and the reaction mixture was partitioned between EtOAc and water. The organic phase was separated and the aqueous phase was extracted with EtOAc (×2). The combined organic extracts were washed with brine, dried over magnesium sulfate, filtered and the solvent was evaporated to dryness. The residue was purified by flash chromatography (hexanes/diethyl ether) to yield the title compound (89 mg, 22%) as a white solid.

MS (m/z): 312 [M+1]⁺

¹H-NMR δ (400 MHz, CDCl₃): 0.87 (t, J=8 Hz, 3H), 1.48-1.13 (m, 15H), 1.68 (m, 2H), 2.77 (t, J=7 Hz, 2H), 4.47-4.31 (m, 2H), 7.35 (s, 1H), 9.10 (s, 1H).

### c) Ethyl 4-decyl-3-fluoro-1H-pyrrole-2-carboxylate

To a solution of ethyl 4-decanoyl-3-fluoro-1H-pyrrole-2-carboxylate (Intermediate 3b, 84 mg, 0.27 mmol) in TFA (2 mL) was added dropwise triethylsilane (0.09 mL, 0.59 mmol) and the mixture was stirred at room temperature for 3 h. TFA was evaporated and the residue was partitioned between DCM and saturated aqueous sodium hydrogen carbonate solution. The organic layer was separated, washed with saturated aqueous sodium hydrogen carbonate solution (×2) and brine, dried over magnesium sulfate, filtered and the solvent was evaporated to dryness. The residue was purified by flash chromatography (hexanes/diethyl ether) to yield the title compound (45 mg, 56%) as a white solid.

MS (m/z): 298 [M+1]⁺

¹H-NMR δ (400 MHz, CDCl₃): 0.92-0.84 (m, 3H), 1.44-1.20 (m, 17H), 1.53 (d, J=7 Hz, 2H), 2.41 (t, J=8 Hz, 2H), 4.33 (q, J=7 Hz, 2H), 6.59-6.53 (m, 1H), 8.37 (s, 1H).

### Intermediate 4

### Ethyl 3-fluoro-4-undecyl-1H-pyrrole-2-carboxylate

### a) Undecanoyl Chloride

Obtained as an oil (100%) from undecanoic acid following the experimental procedure described in Intermediate 3a.

¹H NMR δ (400 MHz, CDCl₃): 0.81-0.94 (m, 3H), 1.28 (d, J=15.0 Hz, 15H), 1.71 (dt, J=15 and 7 Hz, 3H), 2.88 (t, J=7.3 Hz, 2H).

### b) Ethyl 3-fluoro-4-undecanoyl-1H-pyrrole-2-carboxylate

Obtained (59%) from undecanoyl chloride (Intermediate 4a) and ethyl 3-fluoro-1H-pyrrole-2-carboxylate following the experimental procedure described in Intermediate 3b followed by purification of the crude product by reverse phase chromatography (water/ACN both with 0.5% of formic acid).

MS (m/z): 326 [M+1]⁺

¹H NMR δ (400 MHz, CDCl₃): 0.83-0.93 (m, 3H), 1.22-1.45 (m, 17H), 1.68 (p, J=7 Hz, 2H), 2.71-2.82 (m, 2H), 4.38 (q, J=7 Hz, 2H), 7.34 (t, J=4 Hz, 1H), 9.02 (s, 1H).

### c) Ethyl 3-fluoro-4-undecyl-1H-pyrrole-2-carboxylate

Obtained (71%) from ethyl 3-fluoro-4-undecanoyl-1H-pyrrole-2-carboxylate (Intermediate 4b) following the experimental procedure described in Intermediate 3c followed by purification of the crude product by flash chromatography (hexanes/EtOAc).

MS (m/z): 312 [M+1]⁺

¹H NMR δ (400 MHz, CDCl₃) 0.81-0.93 (m, 3H), 1.27 (d, J=14 Hz, 16H), 1.36 (t, J=7 Hz, 3H), 1.49-1.56 (m, 2H), 2.36-2.45 (m, 2H), 4.33 (q, J=7 Hz, 2H), 6.54 (dd, J=4.6 and 3.6 Hz, 1H), 8.38 (s, 1H).

### Intermediate 5

### Ethyl 4-dodecyl-3-fluoro-1H-pyrrole-2-carboxylate

### a) Ethyl 4-dodecanoyl-3-fluoro-1H-pyrrole-2-carboxylate

Obtained (40%) from dodecanoyl chloride and ethyl 3-fluoro-1H-pyrrole-2-carboxylate following the experimental procedure described in Intermediate 3b followed by purification of the crude product by flash chromatography (hexanes/DCM).

MS (m/z): 340 [M+1]⁺

¹H NMR δ (400 MHz, CDCl₃): 0.83-0.93 (m, 3H), 1.17-1.45 (m, 19H), 1.68 (p, J=7.3 Hz, 2H), 2.69-2.82 (m, 2H), 4.38 (q, J=7 Hz, 2H), 7.35 (t, J=4 Hz, 1H), 9.05 (s, 1H).

### b) Ethyl 4-dodecyl-3-fluoro-1H-pyrrole-2-carboxylate

Obtained (62%) from ethyl 4-dodecanoyl-3-fluoro-1H-pyrrole-2-carboxylate (Intermediate 5a) following the experimental procedure described in Intermediate 3c followed by purification of the crude product by flash chromatography (hexanes/DCM).

MS (m/z) 326 [M+1]⁺

¹H NMR δ (400 MHz, CDCl₃) 0.84-0.95 (m, 3H), 1.22-1.33 (m, 18H), 1.36 (t, J=7 Hz, 3H), 1.49-1.55 (m, 2H), 2.36-2.46 (m, 2H), 4.33 (q, J=7 Hz, 2H), 6.53-6.56 (m, 1H), 8.37 (s, 1H).

### Intermediate 6

### 1-Chloroethyl (2-methoxyethyl) carbonate

To a cooled (0° C.) solution of 1-chloroethyl carbonochloridate (250 mg, 1.75 mmol) and 2-methoxyethan-1-ol (121 mg, 1.59 mmol) in DCM (2 mL) pyridine (141 μL, 1.75 mmol) was added dropwise and the resulting mixture was stirred at room temperature for 20 h. The reaction mixture was diluted with DCM and washed with 1N hydrochloric acid solution, water and saturated sodium hydrogen carbonate solution. The organic phase was dried over magnesium sulfate, filtered and solvent was evaporated to dryness to yield the title compound (250 mg, 86%) as a clear oil which was used in the next synthetic step without further purification.

¹H-NMR δ (400 MHz, CDCl₃): 1.83 (d, J=6 Hz, 3H), 3.39 (s, 3H), 3.65-3.61 (m, 2H), 4.34 (ddd, J=7.5 and 4 Hz, 2H), 6.42 (q, J=6 Hz, 1H).

### Intermediate 7

### 1-Chloroethyl (2-(2-ethoxyethoxy)ethyl) carbonate

To a cooled (−78° C.) solution of 2-(2-ethoxyethoxy)ethanol (0.49 mL, 3.5 mmol) and pyridine (0.32 mL, 4.02 mmol) in DCM (5 mL) was slowly added 1-chloroethyl carbonochloridate (0.38 mL, 3.5 mmol) and the reaction mixture was stirred at −78° C. for 3 h. After warming to room temperature, the reaction mixture was filtered and the solid was washed with DCM. The combined organic fractions were washed with water and brine, dried over magnesium sulfate, filtered and the solvent was evaporated to yield the title compound (750 mg, 89%) as a colourless oil which was used in the next synthetic step without further purification.

¹H-NMR δ (400 MHz, CDCl₃): 1.21 (t, J=7 Hz, 3H), 1.83 (d, J=6 Hz, 3H), 3.53 (q, J=7 Hz, 2H), 3.61-3.57 (m, 2H), 3.67-3.63 (m, 2H), 3.77-3.73 (m, 2H), 4.39-4.31 (m, 2H), 6.42 (q, J=6 Hz, 1H).

### Intermediate 8

### Ethyl 3-fluoro-4-tridecanoyl-1H-pyrrole-2-carboxylate

### a) Tridecanoyl Chloride

Obtained as an oil (100%) from tridecanoic acid following the experimental procedure described in Intermediate 3a.

### b) Ethyl 3-fluoro-4-tridecanoyl-1H-pyrrole-2-carboxylate

Obtained as a white solid (68%) from tridecanoyl chloride (Intermediate 8a) and ethyl 3-fluoro-1H-pyrrole-2-carboxylate following the experimental procedure described in Intermediate 3b followed by purification of the crude product by flash chromatography (hexanes/diethyl ether).

MS (m/z): 354 [M+1]⁺

¹H-NMR δ (400 MHz, CDCl₃): 0.96-0.72 (m, 3H), 1.26 (s, 18H), 1.39 (t, J=7 Hz, 3H), 1.68 (p, J=8 Hz, 2H), 2.77 (t, J=8 Hz, 2H), 4.38 (q, J=7 Hz, 2H), 7.35 (s, 1H), 9.03 (brs, 1H).

### Intermediate 9

### Tert-butyl (1-chloroethyl) carbonate

To a cooled (−78° C.) solution of tert-butanol (0.18 mL, 1.84 mmol) and pyridine (0.16 mL, 2.01 mmol) in DCM (3 mL) was slowly added 1-chloroethyl carbonochloridate (0.19 mL, 1.75 mmol) and the mixture was stirred at room temperature overnight. The reaction mixture was then diluted with DCM, washed with 0.5N hydrochloric acid solution and water, dried over magnesium sulfate, filtered and the solvent was evaporated to give the title compound (163 mg, 52%) as a colourless oil which was used in the next synthetic step without further purification.

¹H-NMR δ (400 MHz, CDCl₃): 1.52 (s, 9H), 1.81 (d, J=6 Hz, 3H), 6.39 (q, J=6 Hz, 1H).

### Intermediate 10

### 1-Chloroethyl nonyl carbonate

Obtained as a brown oil (46%) from nonan-1-ol and 1-chloroethyl carbonochloridate following the experimental procedure described in Intermediate 7.

¹H-NMR δ (400 MHz, CDCl₃): 0.80-0.96 (m, 3H), 1.18-1.43 (m, 12H), 1.69 (p, J=7 Hz, 2H), 1.83 (d, J=6 Hz, 3H), 4.20 (t, J=8 Hz, 2H), 6.43 (q, J=6 Hz, 1H).

### Intermediate 11

### Benzyl (1-chloroethyl) carbonate

To a cooled (0° C.) solution of 1-chloroethyl carbonochloridate (250 mg, 1.75 mmol) and phenylmethanol (172 mg, 1.59 mmol) in DCM (5 mL) was added dropwise pyridine (141 μL, 1.75 mmol) and the mixture was stirred at room temperature for 20 h. 1N Hydrochloric acid solution was then added and phases were separated. The organic phase was washed with water and saturated aqueous sodium hydrogen carbonate solution, dried over magnesium sulfate, filtered and solvent was evaporated to yield the title compound (317 mg, 93%) as a clear oil which was used in the next synthetic step without further purification.

¹H-NMR δ (400 MHz, CDCl₃): 1.82 (d, J=6 Hz, 3H), 5.20 (d, J=12 Hz, 1H), 5.24 (d, J=12 Hz, 1H), 6.44 (q, J=6 Hz, 1H), 7.33-7.41 (m, 5H).

### Intermediate 12

### 3-(Benzyloxy)propyl (1-chloroethyl) carbonate

Obtained (98%) from 1-chloroethyl carbonochloridate and 3-(benzyloxy)propan-1-ol following the experimental procedure described in Intermediate 7 followed by purification of the crude product by flash chromatography (hexanes/DCM).

MS (m/z): 273 [M+1]⁺

¹H NMR δ (400 MHz, CDCl₃): 1.75 (d, J=5.8 Hz, 3H), 1.84-2.00 (m, 2H), 3.50 (t, J=6 Hz, 2H), 4.27 (t, J=6 Hz, 2H), 4.44 (s, 3H), 6.25-6.51 (m, 1H), 7.13-7.40 (m, 5H).

### Intermediate 13

### Ethyl 3-fluoro-4-tetradecyl-1H-pyrrole-2-carboxylate

### a) Tetradecanoyl Chloride

Obtained as an oil (100%) from tetradecanoic acid following the experimental procedure described in Intermediate 3a.

### b) Ethyl 3-fluoro-4-tetradecanoyl-1H-pyrrole-2-carboxylate

Obtained (53%) from tetradecanoyl chloride (Intermediate 13a) and ethyl 3-fluoro-1H-pyrrole-2-carboxylate following the experimental procedure described in Intermediate 3b followed by purification of the crude product by reverse phase chromatography (water/ACN both with 0.5% of formic acid).

MS (m/z): 368 [M+1]⁺

¹H NMR δ (400 MHz, CDCl₃): 0.95-0.80 (m, 3H), 1.17-1.50 (m, 23H), 1.68 (p, J=7.4 Hz, 2H), 2.77 (t, J=8.1 Hz, 2H), 4.38 (q, J=7.1 Hz, 2H), 7.35 (t, J=4.1 Hz, 1H), 9.03 (s, 1H).

### c) Ethyl 3-fluoro-4-tetradecyl-1H-pyrrole-2-carboxylate

Obtained (68%) from ethyl 3-fluoro-4-tetradecanoyl-1H-pyrrole-2-carboxylate (Intermediate 13b) following the experimental procedure described in Intermediate 3c followed by purification of the crude product by reverse phase chromatography (water/ACN both with 0.5% of formic acid).

MS (m/z): 354 [M+1]⁺

¹H NMR δ (400 MHz, CDCl₃); 0.88 (t, J=6.8 Hz, 3H), 1.23-1.34 (m, 22H), 1.36 (t, J=7.1 Hz, 2H), 1.47-1.59 (m, 2H), 2.41 (t, J=7.6 Hz, 2H), 4.33 (q, J=7.1 Hz, 2H), 6.43-6.65 (m, 1H), 8.34 (s, 1H).

### Intermediate 14

### Ethyl 3-fluoro-4-pentadecyl-1H-pyrrole-2-carboxylate

### a) Ethyl 3-fluoro-4-pentadecanoyl-1H-pyrrole-2-carboxylate

Obtained as a white solid (100%) from ethyl 3-fluoro-1H-pyrrole-2-carboxylate and pentadecanoyl chloride following the experimental procedure described in Intermediate 3b.

MS (m/z): 382 [M+1]⁺.

¹H-NMR δ (600 MHz, CDCl₃): 0.87 (t, J=7.0 Hz, 3H), 1.21-1.32 (m, 20H), 1.38 (t, J=7.1 Hz, 3H), 1.58-1.72 (m, 3H), 2.35 (t, J=7.5 Hz, 1H), 2.74-2.81 (m, 2H), 4.37 (q, J=7.1 Hz, 2H), 7.36 (t, J=4.0 Hz, 1H), 9.28 (bs, 1H).

### b) Ethyl 3-fluoro-4-pentadecyl-1H-pyrrole-2-carboxylate

Obtained as a white solid (99%) from ethyl 3-fluoro-4-pentadecanoyl-1H-pyrrole-2-carboxylate (Intermediate 14a) following the experimental procedure described in Intermediate 3c followed by purification of the crude product by flash chromatography (hexanes to diethyl ether).

MS (m/z): 368 [M+1]⁺.

¹H-NMR δ (600 MHz, CDCl₃): 0.88 (t, J=7.0 Hz, 3H), 1.24-1.30 (m, 24H), 1.36 (t, J=7.1 Hz, 3H), 1.50-1.59 (m, 2H), 2.36-2.45 (m, 2H), 4.33 (q, J=7.1 Hz, 2H), 6.53-6.56 (m, 1H), 8.35 (bs, 1H).

### Intermediate 15

### Ethyl 3-fluoro-4-heptadecyl-1H-pyrrole-2-carboxylate

### a) Ethyl 3-fluoro-4-heptadecanoyl-1H-pyrrole-2-carboxylate

Obtained as an off-white solid (44%) from ethyl 3-fluoro-1H-pyrrole-2-carboxylate and heptadecanoyl chloride following the experimental procedure described in Intermediate 3b followed by purification by flash chromatography (hexanes to diethyl ether).

MS (m/z): 410 [M+1]⁺.

¹H-NMR δ (400 MHz, CDCl₃): 0.88 (t, J=6.8 Hz, 3H), 1.22-1.32 (m, 26H), 1.39 (t, J=7.1 Hz, 3H), 1.63-1.72 (m, 3H), 2.35 (t, J=7.5 Hz, 1H), 2.71-2.84 (m, 2H), 4.38 (q, J=7.1 Hz, 2H), 7.34 (t, J=4.1 Hz, 1H), 9.03 (bs, 1H).

### b) Ethyl 3-fluoro-4-heptadecyl-1H-pyrrole-2-carboxylate

Obtained as a white solid (58%) from ethyl 3-fluoro-4-heptadecanoyl-1H-pyrrole-2-carboxylate (Intermediate 15a) following the experimental procedure described in Intermediate 3c followed by purification by flash chromatography (hexanes to diethyl ether).

MS (m/z): 397 [M+1]⁺.

¹H-NMR δ (400 MHz, CDCl₃): 0.88 (t, J=6.8 Hz, 3H), 1.21-1.31 (m, 30H), 1.36 (t, J=7.1 Hz, 3H), 2.33-2.48 (m, 2H), 4.33 (q, J=7.1 Hz, 2H), 6.35-6.66 (m, 1H), 8.32 (bs, 1H).

### Intermediate 16

### Ethyl 5-dodecyl-3-fluoro-1H-pyrrole-2-carboxylate

### a) Ethyl 5-dodecanoyl-3-fluoro-1H-pyrrole-2-carboxylate

To a cooled (0° C.) solution of dodecanoyl chloride (1.47 mL, 6.35 mmol) in DCM (10 mL) under an argon atmosphere were added portionwise zinc(II) chloride (867 mg, 6.36 mmol) and a solution of ethyl 3-fluoro-1H-pyrrole-2-carboxylate (500 mg, 3.18 mmol) in DCM (5 mL) and the resulting mixture was stirred at room temperature overnight. The reaction mixture was poured into ice/water and extracted with DCM (×2). The combined organic extracts were washed with saturated aqueous sodium hydrogen carbonate solution and brine, dried over magnesium sulfate, filtered and the solvent was evaporated. The residue was purified by flash chromatography (hexanes/diethyl ether) to yield the title compound (353 mg, 33%) as a yellow solid.

MS (m/z): 340 [M+1]⁺

¹H-NMR δ (400 MHz, CDCl₃): 0.78-1.01 (m, 3H), 1.23-1.41 (m, 21H), 1.61-1.78 (m, 2H), 2.42-2.81 (m, 2H), 4.19-4.52 (m, 2H), 6.22-6.64 (m, 1H), 9.40 (s, 1H).

Ethyl 4-dodecanoyl-3-fluoro-1H-pyrrole-2-carboxylate (Intermediate 5a, 249 mg, 23%) was also isolated from the reaction mixture.

### b) Ethyl 5-dodecyl-3-fluoro-1H-pyrrole-2-carboxylate

To a cooled (0° C.) solution of ethyl 5-dodecanoyl-3-fluoro-1H-pyrrole-2-carboxylate (Intermediate 16a, 343 mg, 1.04 mmol) in TFA (10 mL) were added triethylsilane (500 μL, 3.13 mmol) and boron trifluoride etherate (5.0 mL, 40.5 mmol) and the mixture was stirred at room temperature for 2 h. Water (3 mL) was then added and the reaction mixture was extracted with EtOAc (×3). The combined organic extracts were washed with brine, dried over magnesium sulfate, filtered and the solvent was evaporated. The residue was purified by flash chromatography (hexanes/diethyl ether) to yield the title compound (177 mg, 52%) as a yellow solid.

MS (m/z): 326 [M+1]⁺

¹H-NMR δ (400 MHz, CDCl₃): 0.88 (t, J=7 Hz, 3H), 1.11-1.43 (m, 19H), 1.62 (dt, J=19 and 7 Hz, 2H), 2.36 (t, J=6 Hz, 2H), 2.53 (t, J=8 Hz, 2H), 4.32 (q, J=7 Hz, 1H), 5.62-5.79 (m, 1H), 8.58 (s, 1H).

### Intermediate 17

### Methyl 3-chloro-4-decyl-1H-pyrrole-2-carboxylate

### a) Methyl 3-chloro-4-decanoyl-1H-pyrrole-2-carboxylate

To a cooled (0° C.) solution of decanoyl chloride (Intermediate 3a, 308 mg, 1.62 mmol) in DCM (4 mL) was added aluminium(III) chloride (395 mg, 2.96 mmol) followed by the addition of a solution of methyl 3-chloro-1H-pyrrole-2-carboxylate (215 mg, 1.35 mmol) in DCM (4 mL) and the resulting mixture was stirred at room temperature for three days. 1N Hydrochloric acid solution (1 mL) was then added and the mixture was extracted with EtOAc (×3). The combined organic extracts were washed with water and brine, dried over magnesium sulfate, filtered and the solvent was evaporated to dryness. The residue was purified by flash chromatography (hexanes/diethyl ether) to yield the title compound (300 mg, 71%) as a white solid.

MS (m/z): 314/316 [M+1/M+3]⁺

¹H-NMR δ (400 MHz, CDCl₃): 0.98-0.69 (m, 3H), 1.44-1.18 (m, 12H), 1.69 (p, J=7 Hz, 2H), 2.97-2.76 (m, 2H), 3.93 (s, 3H), 7.52 (d, J=4 Hz, 1H), 9.36 (s, 1H).

### b) Methyl 3-chloro-4-decyl-1H-pyrrole-2-carboxylate

To a solution of methyl 3-chloro-4-decanoyl-1H-pyrrole-2-carboxylate (Intermediate 17a, 295 mg, 0.94 mmol) in TFA (8 mL) was added triethylsilane (0.33 mL, 2.07 mmol) and the mixture was stirred at room temperature for 4 h. The volatiles were removed under reduced pressure and the crude was partitioned between DCM and aqueous saturated sodium hydrogen carbonate solution. The organic phase was separated and washed with aqueous saturated sodium hydrogen carbonate solution, water and brine, dried over magnesium sulfate, filtered and the solvent was evaporated. The residue was purified by flash chromatography (hexanes/diethyl ether) to give the title compound (178 mg, 63%) as a white solid.

MS (m/z): 300/302 [M+1/M+3]⁺

¹H-NMR δ (400 MHz, CDCl₃): 0.96-0.79 (m, 3H), 1.28 (d, J=17 Hz, 14H), 1.56-1.48 (m, 2H), 2.52-2.39 (m, 2H), 3.88 (s, 3H), 6.70 (d, J=3 Hz, 1H), 8.86 (s, 1H).

### Intermediate 18

### Methyl 3-chloro-4-undecyl-1H-pyrrole-2-carboxylate

### a) Methyl 3-chloro-4-undecanoyl-1H-pyrrole-2-carboxylate

Obtained (59%) from undecanoyl chloride (Intermediate 4a) and methyl 3-chloro-1H-pyrrole-2-carboxylate following the experimental procedure as described in Intermediate 17a followed by purification of the crude product by flash chromatography (hexanes/DCM).

MS (m/z): 328,330 [M+1/M+3]⁺

¹H NMR δ (400 MHz, CDCl₃): 0.81-0.95 (m, 3H), 1.17-1.47 (m, 14H), 1.69 (p, J=7 Hz, 2H), 2.81-2.91 (m, 2H), 3.93 (s, 3H), 7.52 (d, J=4 Hz, 1H), 9.37 (s, 1H).

### b) Methyl 3-chloro-4-undecyl-1H-pyrrole-2-carboxylate

Obtained (53%) from methyl 3-chloro-4-undecanoyl-1H-pyrrole-2-carboxylate (Intermediate 18a) following the experimental procedure as described in Intermediate 17b followed by purification of the crude product by flash chromatography (hexanes/diethyl ether) followed by reverse phase chromatography (water/ACN both with 0.5% of formic acid).

MS (m/z): 314, 316 [M+1/M+3]⁺

¹H NMR δ (400 MHz, CDCl₃): 0.83-0.97 (m, 3H), 1.21-1.41 (m, 16H), 1.50-1.59 (m, 2H), 2.40-2.50 (m, 2H), 3.88 (s, 3H), 6.70 (d, J=3 Hz, 1H), 8.86 (s, 1H).

### Intermediate 19

### Methyl 3-chloro-4-dodecyl-1H-pyrrole-2-carboxylate

### a) Methyl 3-chloro-4-dodecanoyl-1H-pyrrole-2-carboxylate

Obtained as a light brown solid (57%) from methyl 3-chloro-1H-pyrrole-2-carboxylate and dodecanoyl chloride following the procedure described in Intermediate 17a. The resulting solid was triturated with hexane, filtered and dried to give the title compound.

MS (m/z): 342 [M+1]⁺.

### b) Methyl 3-chloro-4-dodecyl-1H-pyrrole-2-carboxylate

Obtained as a white solid (88%) from methyl 3-chloro-4-dodecanoyl-1H-pyrrole-2-carboxylate (Intermediate 19a) following the procedure described in Intermediate 17b. The solid was triturated with diethyl ether, filtered and dried to give the title compound.

MS (m/z): 328 [M+1]⁺.

### Intermediate 20

### Methyl 3-chloro-4-tridecyl-1H-pyrrole-2-carboxylate

### a) Methyl 3-chloro-4-tridecanoyl-1H-pyrrole-2-carboxylate

Obtained as white solid (15%) from methyl 3-chloro-1H-pyrrole-2-carboxylate and tridecanoyl chloride (Intermediate 8a) following the experimental procedure described in Intermediate 17a followed by purification by flash chromatography (hexanes to EtOAc).

MS (m/z): 356 [M+1]⁺.

¹H-NMR δ (600 MHz, CDCl₃): 0.87 (t, J=7.1 Hz, 3H), 1.25-1.37 (m, 18H), 1.61-1.71 (m, 2H), 2.84-2.87 (m, 2H), 3.93 (s, 3H), 7.53 (d, J=3.8 Hz, 1H), 9.40 (brs, 1H).

### b) Methyl 3-chloro-4-tridecyl-1H-pyrrole-2-carboxylate

Obtained as a white solid (54%) from methyl 3-chloro-4-tridecanoyl-1H-pyrrole-2-carboxylate (Intermediate 20a) following the experimental procedure described in Intermediate 17b followed by purification by flash chromatography (hexanes to EtOAc).

MS (m/z): 342 [M+1]⁺.

¹H-NMR δ (600 MHz, CDCl₃): 0.87 (t, J=7.02 Hz, 3H), 1.25-1.33 (m, 20H), 1.51-1.55 (m, 2H), 2.42-2.45 (m, 2H), 3.88 (s, 3H), 6.70 (d, J=3.2 Hz, 2H), 8.86 (brs, 1H).

### Intermediate 21

### Methyl 3-chloro-4-pentadecyl-1H-pyrrole-2-carboxylate

### a) Methyl 3-chloro-4-pentadecanoyl-1H-pyrrole-2-carboxylate

Obtained as a white solid (53%) from methyl 3-chloro-1H-pyrrole-2-carboxylate and pentadecanoyl chloride following the procedure described in Intermediate 17a. The crude product was purified using SP1® Purification System (hexanes to diethyl ether) to give the title compound.

MS (m/z): 384 [M+1]⁺.

¹H NMR δ (400 MHz, CDCl₃): 0.99-0.79 (m, 3H), 1.25 (m, 22H), 1.69 (p, J=7 Hz, 2H), 2.94-2.77 (m, 2H), 3.93 (s, 3H), 7.52 (d, J=4 Hz, 1H), 9.36 (brs, 1H).

### b) Methyl 3-chloro-4-pentadecyl-1H-pyrrole-2-carboxylate

Obtained as a white solid (73%) from methyl 3-chloro-4-pentadecanoyl-1H-pyrrole-2-carboxylate (Intermediate 21a) following the procedure described in Intermediate 17b.

MS (m/z): 370 [M+1]⁺.

¹H NMR δ (400 MHz, CDCl₃): 0.99-0.79 (m, 3H), 1.25 (m, 22H), 1.69 (p, J=7 Hz, 2H), 2.94-2.77 (m, 2H), 3.93 (s, 3H), 7.52 (d, J=4 Hz, 1H), 9.36 (brs, 1H).

### Intermediate 22

### Methyl 3-chloro-4-hexadecyl-1H-pyrrole-2-carboxylate

### a) Methyl 3-chloro-4-palmitoyl-1H-pyrrole-2-carboxylate

Obtained as a white solid (61%) from methyl 3-chloro-1H-pyrrole-2-carboxylate and palmitoyl chloride following the procedure described in Intermediate 17a. The crude product was purified using SP1® Purification System (hexanes to EtOAc) to give the title compound.

MS (m/z): 398 [M+1]⁺.

¹H NMR δ (400 MHz, CDCl₃): 0.87 (t, J=7 Hz, 3H), 1.38-1.22 (m, 24H), 1.69 (p, J=7 Hz, 2H), 2.86 (t, J=7 Hz, 2H), 3.93 (s, 3H), 7.52 (d, J=4 Hz, 1H), 9.38 (brs, 1H).

### b) Methyl 3-chloro-4-hexadecyl-1H-pyrrole-2-carboxylate

Obtained as a solid (71%) from methyl 3-chloro-4-palmitoyl-1H-pyrrole-2-carboxylate (Intermediate 22a) following the procedure described in Intermediate 17b.

MS (m/z): 384 [M+1]⁺.

¹H NMR δ (400 MHz, CDCl₃): 0.88 (t, J=7 Hz, 3H), 1.35-1.23 (m, 26H), 1.58-1.50 (m, 2H), 2.44 (t, J=8 Hz, 2H), 3.88 (s, 3H), 6.70 (d, J=3 Hz, 1H), 8.86 (brs, 1H).

### Intermediate 23

### Methyl 3-chloro-5-undecyl-1H-pyrrole-2-carboxylate

### a) Methyl 3-chloro-5-undecanoyl-1H-pyrrole-2-carboxylate

To a solution of undecanoyl chloride (Intermediate 4a, 512 mg, 2.5 mmol) in DCE (4 mL) was added zinc(II) chloride (342 mg, 2.5 mmol) and the mixture was cooled to 0° C. Then a solution of methyl 3-chloro-1H-pyrrole-2-carboxylate (200 mg, 1.25 mmol) in DCE (4 mL) was added and the mixture was stirred at room temperature for 18 h. Ice and DCM were added to the reaction mixture and phases were separated. The organic phase was washed with aqueous saturated sodium hydrogen carbonate solution and brine, dried over magnesium sulfate, filtered and solvent evaporated. Purification of the residue by flash chromatography (hexanes/EtOAc) gave the title compound (100 mg, 24%) as a yellow solid.

MS (m/z): 328/330 [M+1]⁺.

¹H-NMR δ (400 MHz, CDCl₃): 0.88 (t, J=7 Hz, 3H), 1.18-1.38 (14H), 1.70 (p, J=7 Hz, 2H), 2.75 (t, J=7 Hz, 2H), 3.93 (s, 3H), 6.80 (d, J=3 Hz, 1H), 9.80 (brs, 1H).

### b) Methyl 3-chloro-5-undecyl-1H-pyrrole-2-carboxylate

To a solution of methyl 3-chloro-5-undecanoyl-1H-pyrrole-2-carboxylate (Intermediate 23a, 100 mg, 0.3 mmol) in TFA (4 mL) was added triethylsilane (190 μL, 1.19 mmol) and the mixture was stirred at room temperature for 20 h. Solvent was removed and the residue was purified by flash chromatography (hexanes/EtOAc) to yield the title compound (30 mg, 32%) as an off white solid.

MS (m/z): 314/316 [M+1]⁺.

¹H-NMR δ (400 MHz, CDCl₃): 0.88 (t, J=7 Hz, 3H), 1.22-1.38 (16H), 1.60 (p, J=7 Hz, 2H), 2.54 (t, J=7 Hz, 2H), 3.87 (s, 3H), 5.97 (d, J=3 Hz, 1H), 8.76 (brs, 1H).

### Intermediate 24

### Ethyl 3-chloro-5-dodecyl-1H-pyrrole-2-carboxylate

### a) 1-(1H-Pyrrol-2-yl)dodecan-1-one

To a solution of 1H-pyrrole (1.0 g, 14.9 mmol) and dodecanoyl chloride (4.14 mL, 17.9 mmol) in toluene (24 mL) was added zinc (1.95 g, 29.8 mmol) and the mixture was stirred at room temperature for 2 h. The reaction mixture was then partitioned between aqueous saturated sodium hydrogen carbonate solution and EtOAc. The organic layer was separated, washed with water, dried over magnesium sulfate, filtered and the solvent was evaporated. Purification of the residue by flash chromatography (hexanes/EtOAc) gave the title compound (1.6 g, 42%) as a dark brown solid.

MS (m/z): 250 [M+1]⁺.

¹H-NMR δ (400 MHz, CDCl₃): 0.88 (t, J=7 Hz, 3H), 1.18-1.39 (m, 16H), 1.71 (p, J=8 Hz, 2H), 2.75 (t, J=8 Hz, 2H), 6.27 (dt, J=4 and 3 Hz, 1H), 6.92 (ddd, J=4, 3 and 1 Hz, 1H), 7.03 (td, J=3 and 1 Hz, 1H), 9.63 (brs, 1H).

### b) 2-Dodecyl-1H-pyrrole

To a suspension of 1-(1H-pyrrol-2-yl)dodecan-1-one (Intermediate 24a, 1.6 g, 6.34 mmol) in diethylenglycol (24 mL) were added potassium hydroxide (4.8 g, 86 mmol) and hydrazine hydrate (7.3 g, 146 mmol) and the mixture was heated at 200° C. for 2 h. The reaction mixture was cooled down and water was added. The precipitate formed was filtered off, washed with water and dried in a vacuum oven to yield the title compound (1.15 g, 77%) as a light brown solid.

MS (m/z): 236 [M+1]⁺.

¹H-NMR δ (400 MHz, CDCl₃): 0.88 (t, J=7 Hz, 3H), 1.22-1.39 (m, 18H), 1.62 (p, J=8 Hz, 2H), 2.59 (t, J=8 Hz, 2H), 5.90-5.91 (m, 1H), 6.11-6.14 (m, 1H), 6.65-6.67 (mz, 1H), 7.88 (brs, 1H).

### c) 2,2,2-Trichloro-1-(5-dodecyl-1H-pyrrol-2-yl)ethan-1-one

A mixture of 2-dodecyl-1H-pyrrole (Intermediate 24b, 1.15 g, 4.9 mmol), 2,6-lutidine (683 μL, 5.86 mmol) and 2,2,2-trichloroacetyl chloride (654 μL, 5.86 mmol) in dioxane (6 mL) was heated at 85° C. for 16 h. After cooling to room temperature, the reaction mixture was partitioned between EtOAc and 1N hydrochloric acid solution. The organic phase was separated, washed with brine, dried over magnesium sulfate, filtered and solvent was evaporated. Purification of the residue by flash chromatography (hexanes/diethyl ether) gave the title compound (948 mg, 51%) as a light brown solid.

MS (m/z): 380/382/384 [M+1/M+3/M+5]⁺.

¹H-NMR δ (400 MHz, CDCl₃): 0.88 (t, J=7 Hz, 3H), 1.22-1.39 (m, 18H), 1.67 (p, J=7 Hz, 2H), 2.67 (t, J=8 Hz, 2H), 6.10-6.12 (m, 1H), 7.32 (dd, J=4 and 2 Hz, 1H), 9.19 (brs, 1H).

### d) Ethyl 5-dodecyl-1H-pyrrole-2-carboxylate

To a solution of sodium (68.2 mg, 2.97 mmol) in ethanol (25 mL) was added 2,2,2-trichloro-1-(5-dodecyl-1H-pyrrol-2-yl)ethan-1-one (Intermediate 24c, 942 mg, 2.47 mmol) and the dark brown solution was stirred at room temperature for 30 min. Ethanol was removed and the mixture was partitioned between 1N hydrochloric acid solution and EtOAc. The organic phase was separated, dried over magnesium sulfate, filtered and solvent was evaporated to yield the title compound (680 mg, 89%) as a brown solid.

MS (m/z): 308 [M+1]⁺.

¹H-NMR δ (400 MHz, CDCl₃): 0.88 (t, J=7 Hz, 3H), 1.22-1.37 (m, 21H), 1.62 (p, J=8 Hz, 2H), 2.60 (t, J=8 Hz, 2H), 4.29 (q, J=7 Hz, 2H), 5.96 (dd, J=4 and 3 Hz, 1H), 6.82 (dd, J=4 and 3 Hz, 1H), 8.84 (brs, 1H).

### e) Ethyl 3-chloro-5-dodecyl-1H-pyrrole-2-carboxylate

To a solution of ethyl 5-dodecyl-1H-pyrrole-2-carboxylate (Intermediate 24d, 380 mg, 1.24 mmol) in chloroform (6 mL) was added NCS (165 mg, 1.24 mmol) and the mixture was heated at 40° C. for 1 h. The reaction mixture was cooled down and poured into a cooled (0° C.) aqueous 5% sodium hydroxide solution. Additional chloroform was added and phases were separated. The organic phase was washed with brine, dried over magnesium sulfate, filtered and solvent was evaporated. Purification of the residue by reverse chromatography (water/ACN both containing 0.01% of formic acid) gave ethyl 3-chloro-5-dodecyl-1H-pyrrole-2-carboxylate (40 mg, 9%) as a white solid.

MS (m/z): 342/344 [M+1/M+3]⁺.

¹H-NMR δ (400 MHz, CDCl₃): 0.88 (t, J=7 Hz, 3H), 1.22-1.34 (m, 18H), 1.37 (t, J=7 Hz, 3H), 1.55-1.63 (m, 2H), 2.55 (t, J=8 Hz, 2H), 4.34 (q, J=7 Hz, 2H), 5.96 (d, J=3 Hz, 1H), 8.89 (brs, 1H).

and ethyl 4-chloro-5-dodecyl-1H-pyrrole-2-carboxylate (200 mg, 59%) as a white solid.

MS (m/z): 342/344 [M+1/M+3]⁺.

¹H-NMR δ (400 MHz, CDCl₃): 0.88 (t, J=7 Hz, 3H), 1.21-1.35 (m, 18H), 1.34 (t, J=7 Hz, 3H), 1.61 (p, J=7 Hz, 2H), 2.61 (t, J=8 Hz, 2H), 4.30 (q, J=7 Hz, 2H), 6.76 (d, J=3 Hz, 1H), 8.85 (brs, 1H).

### Intermediate 25

### Methyl 3-chloro-5-tridecyl-1H-pyrrole-2-carboxylate

### a) Methyl 3-chloro-5-tridecanoyl-1H-pyrrole-2-carboxylate

Obtained as a yellow solid (20%) from tridecanoyl chloride (Intermediate 8a) and methyl 3-chloro-1H-pyrrole-2-carboxylate following the procedure described in Intermediate 23a followed by purification of the crude product by flash chromatography (hexanes/diethyl ether).

MS (m/z): 356/358 [M+1, Cl]⁺

¹H NMR δ (400 MHz, CDCl₃): 0.85-0.91 (m, 3H), 1.18-1.34 (m, 18H), 1.66-1.74 (m, 2H), 2.71-2.78 (m, 2H), 3.93 (s, 3H), 6.79 (s, 1H), 9.76 (s, 1H).

Its regioisomer 3-chloro-4-tridecanoyl-1H-pyrrole-2-carboxylate (Intermediate 20a, 34%) was also isolated in this reaction.

### b) Methyl 3-chloro-5-tridecyl-1H-pyrrole-2-carboxylate

Obtained as a white solid (52%) from methyl 3-chloro-5-tridecanoyl-1H-pyrrole-2-carboxylate (Intermediate 25a) following the procedure described in Intermediate 23b followed by purification of the crude product by flash chromatography (hexanes/diethyl ether and then hexanes/EtOAc).

MS (m/z): 342/344 [M+1, Cl]⁺

¹H NMR δ (400 MHz, CDCl₃): 0.87-0.92 (m, 3H), 1.15-1.26 (m, 20H), 1.52-1.67 (m, 2H), 2.55 (t, J=8 Hz, 2H), 3.87 (s, 3H), 5.97 (d, J=3 Hz, 1H), 8.76 (s, 1H).

### Intermediate 26

### Methyl 3-chloro-5-tetradecyl-1H-pyrrole-2-carboxylate

### a) Methyl 3-chloro-5-tetradecanoyl-1H-pyrrole-2-carboxylate

Obtained as a pink solid (21%) from tetradecanoyl chloride (Intermediate 13a) and methyl 3-chloro-1H-pyrrole-2-carboxylate following the procedure described in Intermediate 23a followed by purification of the crude product by flash chromatography (hexanes/EtOAc).

MS (m/z): 370/372 [M+1]⁺.

¹H-NMR δ (400 MHz, CDCl₃): 0.88 (t, J=7 Hz, 3H), 1.20-1.40 (m, 20H), 1.70 (p, J=7 Hz, 2H), 2.75 (t, J=7 Hz, 2H), 3.93 (s, 3H), 6.79 (d, J=3 Hz, 1H), 9.74 (brs, 1H).

### b) Methyl 3-chloro-5-tetradecyl-1H-pyrrole-2-carboxylate

Obtained as an off white solid (33%) from methyl 3-chloro-5-tetradecanoyl-1H-pyrrole-2-carboxylate (Intermediate 26a) following the experimental procedure described in Intermediate 23b followed by purification of the crude product by flash chromatography (hexanes/EtOAc).

MS (m/z): 356/358 [M+1]⁺.

¹H-NMR δ (400 MHz, CDCl₃): 0.87 (t, J=7 Hz, 3H), 1.20-1.36 (m, 22H), 1.60 (p, J=7 Hz, 2H), 2.54 (t, J=7 Hz, 2H), 3.87 (s, 3H), 5.97 (d, J=3 Hz, 1H), 8.71 (brs, 1H).

### Intermediate 27

### Methyl 3-bromo-4-tridecyl-1H-pyrrole-2-carboxylate

### a) Methyl 3-bromo-4-tridecanoyl-1H-pyrrole-2-carboxylate

Obtained as a white solid (54%) from methyl 3-bromo-1H-pyrrole-2-carboxylate and tridecanoyl chloride (Intermediate 8a) following the procedure described in Intermediate 3b.

MS (m/z): 400, 402 [M+1]⁺

### b) Methyl 3-bromo-4-tridecyl-1H-pyrrole-2-carboxylate

Obtained as a solid (85%) from methyl 3-bromo-4-tridecanoyl-1H-pyrrole-2-carboxylate (Intermediate 27a) following the procedure described in Intermediate 3c.

MS (m/z): 384, 386 [M−1]⁺.

¹H NMR δ (400 MHz, CDCl₃): 0.92-0.82 (m, 3H), 1.26 (m, 20H), 1.55 (p, J=7.4 Hz, 2H), 2.55-2.29 (m, 2H), 3.89 (s, 3H), 6.73 (d, J=3.2 Hz, 1H), 9.16 (s, 1H).

### Intermediate 28

### Ethyl 4-(decyloxy)-3-fluoro-1H-pyrrole-2-carboxylate

### a) Ethyl 4-(2-chloroacetyl)-3-fluoro-1H-pyrrole-2-carboxylate

Obtained as a grey solid (95%) from ethyl 3-fluoro-1H-pyrrole-2-carboxylate and 2-chloroacetyl chloride following the experimental procedure described in Intermediate 1a.

MS (m/z): 234, 236 [M+1, M+3]⁺.

¹H-NMR δ (600 MHz, CDCl₃): 1.39 (t, J=7.1 Hz, 3H), 4.39 (q, J=7.1 Hz, 2H), 4.53 (s, 2H), 7.47 (t, J=4.0 Hz, 1H), 9.35 (bs, 1H).

### b) Ethyl 4-(2-chloroacetoxy)-3-fluoro-1H-pyrrole-2-carboxylate

Obtained as a white solid (34%) from ethyl 4-(2-chloroacetyl)-3-fluoro-1H-pyrrole-2-carboxylate (Intermediate 28a) following the experimental procedure described in Intermediate 1b.

MS (m/z): 250, 252 [M+1, M+3]⁺.

¹H-NMR δ (400 MHz, CDCl₃): 1.37 (t, J=7.1 Hz, 3H), 4.31 (s, 2H), 4.36 (q, J=7.1 Hz, 2H), 6.96 (t, J=3.8 Hz, 1H), 8.64 (bs, 1H).

### c) Ethyl 3-fluoro-4-hydroxy-1H-pyrrole-2-carboxylate

Obtained as a light purple solid (77%) from ethyl 4-(2-chloroacetoxy)-3-fluoro-1H-pyrrole-2-carboxylate (Intermediate 28b) following the experimental procedure described in Intermediate 1c.

MS (m/z): 174 [M+1]⁺.

¹H-NMR δ (400 MHz, CDCl₃): 1.36 (t, J=7.1 Hz, 3H), 4.34 (q, J=7.1 Hz, 2H), 4.64 (bs, 1H), 6.41-6.56 (m, 1H), 8.29 (bs, 1H).

### d) Ethyl 4-(decyloxy)-3-fluoro-1H-pyrrole-2-carboxylate

Obtained as a colourless oil (31%) from ethyl 3-fluoro-4-hydroxy-1H-pyrrole-2-carboxylate (Intermediate 28c) and 1-bromodecane following the experimental procedure described in Intermediate 1d followed by purification by flash chromatography (hexanes to diethyl ether).

MS (m/z): 314 [M+1]⁺.

¹H-NMR δ (400 MHz, CDCl₃): 0.85-0.91 (m, 3H), 1.23-1.33 (m, 12H), 1.36 (t, J=7.1 Hz, 3H), 1.73 (dt, J=14.7, 6.6 Hz, 3H), 3.64 (t, J=6.6 Hz, 2H), 3.91 (t, J=6.6 Hz, 2H), 4.34 (q, J=7.1 Hz, 2H), 6.41-6.47 (m, 1H), 8.09 (bs, 1H).

### Intermediate 29

### Ethyl 3-fluoro-4-(undecyloxy)-1H-pyrrole-2-carboxylate

Obtained as a colourless oil (29%) from ethyl 3-fluoro-4-hydroxy-1H-pyrrole-2-carboxylate (Intermediate 28c) and 1-bromoundecane following the experimental procedure described in Intermediate 1d followed by purification of the crude product by flash chromatography (hexanes to diethyl ether).

MS (m/z): 328 [M+1]⁺.

¹H-NMR δ (400 MHz, CDCl₃): 0.84-0.92 (m, 3H), 1.23-1.33 (m, 16H), 1.36 (t, J=7.1 Hz, 3H), 1.67-1.78 (m, 2H), 3.91 (t, J=6.6 Hz, 2H), 4.34 (q, J=7.1 Hz, 2H), 6.40-6.49 (m, 1H), 8.13 (bs, 1H).

### Intermediate 30

### Ethyl 4-(dodecyloxy)-3-fluoro-1H-pyrrole-2-carboxylate

Obtained as a colourless oil (26%) from ethyl 3-fluoro-4-hydroxy-1H-pyrrole-2-carboxylate (Intermediate 28c) and 1-bromododecane following the experimental procedure described in Intermediate 1d followed by purification of the crude product by flash chromatography (hexanes to diethyl ether).

MS (m/z): 342 [M+1]⁺.

¹H-NMR δ (600 MHz, CDCl₃): 0.88 (t, J=7.1 Hz, 3H), 1.24-1.31 (m, 16H), 1.36 (t, J=7.1 Hz, 3H), 1.41 (m, 2H), 1.70-1.76 (m, 2H), 3.91 (t, J=6.6 Hz, 2H), 4.34 (q, J=7.1 Hz, 2H), 6.43-6.46 (m, 1H), 8.05 (bs, 1H).

### Intermediate 31

### Ethyl 3-fluoro-4-(tridecyloxy)-1H-pyrrole-2-carboxylate

Obtained as a colourless oil (41%) from ethyl 3-fluoro-4-hydroxy-1H-pyrrole-2-carboxylate (Intermediate 28c) and 1-bromotridecane following the experimental procedure described in Intermediate 1d followed by purification of the crude product by flash chromatography (hexanes/DCM).

MS (m/z): 356 [M+1]⁺

¹H NMR δ (400 MHz, CDCl₃): 0.85-0.90 (m, 3H), 1.22-1.39 (m, 16H), 1.39-1.46 (m, 2H), 1.67-1.78 (m, 2H), 3.91 (t, J=6.6 Hz, 2H), 4.34 (q, J=7.1 Hz, 2H), 6.39-6.48 (m, 1H), 8.09 (s, 1H).

### Intermediate 32

### Ethyl 3-fluoro-4-(tetradecyloxy)-1H-pyrrole-2-carboxylate

Obtained as a colourless oil (29%) from ethyl 3-fluoro-4-hydroxy-1H-pyrrole-2-carboxylate (Intermediate 28c) and 1-bromotetradecane following the experimental procedure described in Intermediate 1d followed by purification of the crude product by flash chromatography (hexanes to diethyl ether).

MS (m/z): 368 [M−1]⁺.

¹H-NMR δ (400 MHz, CDCl₃): 0.88 (t, J=6.9 Hz, 3H), 1.23-1.34 (m, 20H), 1.36 (t, J=7.1 Hz, 3H), 1.39-1.45 (m, 2H), 1.73 (dt, J=14.6, 6.6 Hz, 2H), 3.91 (t, J=6.6 Hz, 2H), 4.34 (q, J=7.1 Hz, 2H), 6.41-6.47 (m, 1H), 8.07 (bs, 1H).

### Intermediate 33

### Ethyl 4-(dodecylthio)-3-fluoro-1H-pyrrole-2-carboxylate

### a) Ethyl 3-fluoro-4-thiocyanato-1H-pyrrole-2-carboxylate

To a cooled (−78° C.) suspension of potassium thiocyanate (865 mg, 8.9 mmol) in methanol (2 mL) was added a solution of bromine (229 μL, 4.45 mmol) in methanol (3 mL). The temperature was allowed to rise to −30° C. and a solution of ethyl 3-fluoro-1H-pyrrole-2-carboxylate (700 mg, 4.45 mmol) in methanol (3 mL) was added dropwise. The temperature was allowed to rise to 20° C. and the reaction mixture was poured into cold water (60 mL). The resulting cloudy solution was kept at −5° C. for 1 h and the precipitate formed was filtered off, washed with water and dried. Purification of the solid by flash chromatography (hexanes/diethyl ether) and reverse phase chromatography (water/methanol) gave the title compound (280 mg, 29%) as a white solid.

MS (m/z): 213 [M−1]⁻.

¹H-NMR δ (400 MHz, CDCl₃): 1.39 (t, J=7.1 Hz, 3H), 4.39 (q, J=7.1 Hz, 2H), 7.09 (t, J=3.8 Hz, 1H), 9.14 (bs, 1H).

### b) Ethyl 4-(dodecylthio)-3-fluoro-1H-pyrrole-2-carboxylate

To a stirred mixture of ethyl 3-fluoro-4-thiocyanato-1H-pyrrole-2-carboxylate (Intermediate 33a, 99 mg, 0.461 mmol) and 1-bromododecane (115 mg, 0.461 mmol) in tert-butanol (1 mL) was added an aqueous 4N sodium hydroxide solution (0.268 mL, 1.072 mmol) and the resulting mixture was heated at 60° C. for 4 h. After cooling to room temperature, the solvent was evaporated and the residue was partitioned between water and DCM. The organic phase was separated, washed with brine, dried over magnesium sulfate, filtered and the solvent was evaporated to dryness. Purification of the residue by flash chromatography (hexane/EtOAc) gave the title compound (60 mg, 36%) as a colourless oil.

MS (m/z): 356 [M−1]⁻.

¹H-NMR δ (400 MHz, CDCl₃): 0.88 (t, J=6.9 Hz, 3H), 1.21-1.30 (m, 18H), 1.37 (t, J=7.1 Hz, 3H), 1.48-1.55 (m, 2H), 2.59-2.69 (m, 2H), 4.35 (q, J=7.1 Hz, 2H), 6.83 (t, J=3.9 Hz, 1H), 8.68 (bs, 1H).

### Intermediate 34

### Methyl 3-chloro-4-(nonyloxy)-1H-pyrrole-2-carboxylate

### a) Methyl 3-chloro-4-(2-chloroacetyl)-1H-pyrrole-2-carboxylate

Obtained (84%) from 2-chloroacetyl chloride and methyl 3-chloro-1H-pyrrole-2-carboxylate following the experimental procedure described in Intermediate 1a.

MS (m/z): 236, 238 [M+1, M+3]⁺

¹H NMR δ (400 MHz, DMSO-d6): 3.83 (s, 3H), 4.86 (s, 2H), 7.97 (d, J=3.9 Hz, 1H), 12.89 (s, 1H)

### b) Methyl 3-chloro-4-(2-chloroacetoxy)-1H-pyrrole-2-carboxylate

Obtained (49%) from methyl 3-chloro-4-(2-chloroacetyl)-1H-pyrrole-2-carboxylate (Intermediate 34a) following the experimental procedure described in Intermediate 1b followed by purification of the crude product by flash chromatography (hexanes/EtOAc).

MS (m/z): 252, 354 [M+1, M+3]⁺

¹H NMR δ (400 MHz, CDCl₃): 3.92 (s, 3H), 4.33 (s, 2H), 7.12 (d, J=3.6 Hz, 1H), 9.03 (s, 1H).

### c) Methyl 3-chloro-4-hydroxy-1H-pyrrole-2-carboxylate

Obtained (82%) from methyl 3-chloro-4-(2-chloroacetoxy)-1H-pyrrole-2-carboxylate (Intermediate 34b) following the experimental procedure described in Intermediate 1c followed by purification of the crude product by flash chromatography (hexanes/EtOAc).

MS (m/z): 176,178 [M+1, M+3]⁺

¹H NMR δ (400 MHz, CDCl₃): 3.89 (s, 3H), 6.61 (d, J=3.4 Hz, 1H), 8.66 (s, 1H).

### d) Methyl 3-chloro-4-(nonyloxy)-1H-pyrrole-2-carboxylate

Obtained (9%) from 1-bromononane and methyl 3-chloro-4-hydroxy-1H-pyrrole-2-carboxylate (Intermediate 34c) following the experimental procedure described in Intermediate 1d followed by purification of the crude product by flash chromatography (hexanes/diethyl ether).

¹H NMR δ (400 MHz, CDCl₃) 0.81 (t, J=6.8 Hz, 3H), 1.16-1.42 (m, 12H), 1.70 (p, J=6.8 Hz, 2H), 3.77-3.86 (m, 5H), 6.45 (d, J=3.4 Hz, 1H), 8.54 (s, 1H).

### Intermediate 35

### Methyl 3-chloro-4-(decyloxy)-1H-pyrrole-2-carboxylate

Obtained as a brown solid (18%) from methyl 3-chloro-4-hydroxy-1H-pyrrole-2-carboxylate (Intermediate 34c) and 1-bromodecane following the experimental procedure described in Intermediate 1d followed by purification of the crude product by flash chromatography (hexanes/diethyl ether).

MS (m/z): 316 [M+1]⁺.

¹H-NMR δ (400 MHz, CDCl₃): 0.88 (t, J=6.8 Hz, 3H), 1.21-1.36 (m, 16H), 1.39-1.49 (m, 2H), 1.73-1.81 (m, 2H), 3.89 (s, 3H), 6.52 (d, J=3.5 Hz, 1H), 8.59 (bs, 1H).

### Intermediate 36

### Methyl 3-chloro-4-(undecyloxy)-1H-pyrrole-2-carboxylate

Obtained as a colourless oil (23%) from methyl 3-chloro-4-hydroxy-1H-pyrrole-2-carboxylate (Intermediate 34c) and 1-bromoundecane following the experimental procedure described in Intermediate 1d followed by purification of the crude product by flash chromatography (hexanes/diethyl ether).

MS (m/z): 328 [M−1]⁺.

¹H-NMR δ (400 MHz, CDCl₃): 0.84-0.91 (m, 3H), 1.22-1.37 (m, 16H), 1.39-1.49 (m, 2H), 1.72-1.82 (m, 2H), 3.89 (s, 3H), 6.52 (d, J=3.5 Hz, 1H), 8.59 (bs, 1H).

### Intermediate 37

### Methyl 3-chloro-4-(dodecyloxy)-1H-pyrrole-2-carboxylate

Obtained as an orange solid (46%) from methyl 3-chloro-4-hydroxy-1H-pyrrole-2-carboxylate (Intermediate 34c) and 1-bromododecane following the experimental procedure described in Intermediate 1d followed by purification of the crude product by flash chromatography (hexanes/diethyl ether).

MS (m/z): 342 [M−1]⁻.

¹H-NMR δ (400 MHz, CDCl₃): 0.85-0.91 (m, 3H), 1.21-1.36 (m, 18H), 1.39-1.48 (m, 2H), 1.72-1.82 (m, 2H), 3.89 (s, 3H), 6.52 (d, J=3.5 Hz, 1H), 8.60 (bs, 1H).

### Intermediate 38

### Methyl 3-chloro-4-(tridecyloxy)-1H-pyrrole-2-carboxylate

Obtained as a white solid (37%) from methyl 3-chloro-4-hydroxy-1H-pyrrole-2-carboxylate (Intermediate 34c) and 1-bromotridecane following the experimental procedure as described in Intermediate 1d.

MS (m/z): 358/360 [M+1/M+3]⁺.

¹H-NMR δ (400 MHz, CDCl₃): 0.88 (t, J=7 Hz, 3H), 1.21-1.38 (m, 18H), 1.39-1.47 (m, 2H), 1.73-1.80 (m, 2H), 3.87-3.91 (m, 5H), 6.52 (d, J=4 Hz, 1H), 8.60 (br s, 1H).

### Intermediate 39

### Methyl 3-chloro-4-(tetradecyloxy)-1H-pyrrole-2-carboxylate

Obtained as a white solid (35%) from methyl 3-chloro-4-hydroxy-1H-pyrrole-2-carboxylate (Intermediate 34c) and 1-bromotetradecane following the experimental procedure as described in Intermediate 1d followed by purification of the crude product by flash chromatography (hexanes/diethyl ether).

1H-NMR δ (400 MHz, CDCl₃): 0.87 (t, J=7 Hz, 3H), 1.26 (s, 20H), 1.39-1.48 (m, 2H), 1.77 (p, J=7 Hz, 2H), 3.79-3.97 (m, 5H), 6.45-6.59 (m, 1H), 8.59 (s, 1H).

### Intermediate 40

### Ethyl 4-(12-bromododecyl)-3-fluoro-1H-pyrrole-2-carboxylate

### a) 12-Bromododecanoyl Chloride

To a solution of 12-bromododecanoic acid (100 mg, 0.36 mmol) in DCM (2 mL) was added DMF (1 drop) followed by oxalyl chloride (0.03 mL, 0.36 mmol) and the mixture was stirred at room temperature for 16 h. The volatiles were removed under reduced pressure to yield the title compound (120 mg, 100%) as a yellow-orange oil which was used in the next step synthetic step without further purification.

### b) Ethyl 4-(12-bromododecanoyl)-3-fluoro-1H-pyrrole-2-carboxylate

Obtained as a white solid (17%) from 12-bromododecanoyl chloride (Intermediate 40a) and ethyl 3-fluoro-1H-pyrrole-2-carboxylate following the experimental procedure as described in Intermediate 3b followed by purification of the crude product by flash chromatography (hexanes/diethyl ether).

MS (m/z): 418/420 [M+1/M+3]⁺

¹H-NMR δ (400 MHz, CDCl₃): 1.48-1.17 (m, 17H), 1.74-1.64 (m, 2H), 1.85 (dt, J=15 and 7 Hz, 2H), 2.77 (t, J=8 Hz, 2H), 3.41 (t, J=7 Hz, 2H), 4.38 (q, J=7 Hz, 2H), 7.35 (t, J=4 Hz, 1H).

### c) Ethyl 4-(12-bromododecyl)-3-fluoro-1H-pyrrole-2-carboxylate

Obtained as a white solid (50%) from ethyl 4-(12-bromododecanoyl)-3-fluoro-1H-pyrrole-2-carboxylate (Intermediate 40b) following the experimental procedure as described in Intermediate 3c followed by purification of the crude product by flash chromatography (hexanes/diethyl ether).

MS (m/z): 405/407 [M+1/M+3]⁺

¹H-NMR δ (400 MHz, CDCl₃): 1.40-1.15 (m, 17H), 1.47-1.37 (m, 2H), 1.56-1.48 (m, 2H), 1.85 (dt, J=15 and 7 Hz, 2H), 2.41 (t, J=8 Hz, 2H), 3.41 (t, J=7 Hz, 2H), 4.33 (q, J=7 Hz, 2H), 6.61-6.50 (m, 1H), 8.39 (brs, 1H).

### Intermediate 41

### Ethyl 3-fluoro-4-(2-fluorotridecyl)-1H-pyrrole-2-carboxylate

### a) Ethyl 3-fluoro-4-(2-fluorotridecanoyl)-1H-pyrrole-2-carboxylate

To a cooled (−78° C.) solution of diisopropylamine (0.77 mL, 5.38 mmol) in THF (5 mL) was added dropwise n-butyl lithium (1.6M solution in hexanes, 3.36 mL, 5.38 mmol) and the resulting mixture was stirred at −78° C. for 30 minutes. A solution of ethyl 3-fluoro-4-tridecanoyl-1H-pyrrole-2-carboxylate (Intermediate 8b, 500 mg, 1.41 mmol) in THF (8 mL) was added dropwise and the mixture was stirred for 1 h at −40° C. After cooling again to −78° C., a solution of N-fluorobenzenesulfonimide (1338 mg, 4.24 mmol) in THF (2 mL) was added dropwise and the mixture was stirred at −78° C. for 2 h and then allowed to warm to room temperature. After stirring for 3 h at room temperature, water was slowly added and the reaction mixture was acidified to pH=2-3 by addition of 1N hydrochloric acid solution. The reaction mixture was extracted with EtOAc (×3) and the combined organic extracts were washed with brine, dried over magnesium sulfate, filtered and the solvent was evaporated. The residue was purified by flash chromatography (hexanes/diethyl ether) to yield the title product (340 mg, 65%) as a white solid

MS (m/z): 372 [M+1]⁺

¹H-NMR δ (400 MHz, CDCl₃): 0.87 (t, J=7 Hz, 3H), 1.27 (s, 18H), 1.40 (t, J=7 Hz, 3H), 1.59-1.48 (m, 2H), 2.09-1.78 (m, 2H), 4.39 (q, J=7 Hz, 2H), 5.24 (ddd, J=50, 8 and 4 Hz, 1H), 7.53 (s, 1H), 9.47 (s, 1H).

### b) Ethyl 3-fluoro-4-(2-fluoro-1-hydroxytridecyl)-1H-pyrrole-2-carboxylate

To a cooled (0° C.) solution of ethyl 3-fluoro-4-(2-fluorotridecanoyl)-1H-pyrrole-2-carboxylate (Intermediate 41a, 310 mg, 0.83 mmol) in ethanol (5 mL) was added portionwise sodium borohydride (31.6 mg, 0.83 mmol) and the resulting mixture was stirred at 0° C. for 1 h. Aqueous saturated ammonium chloride solution was then added and the reaction mixture was extracted with EtOAc (×3). The combined organic fractions were washed with brine, dried over magnesium sulfate, filtered and the solvent was evaporated. The residue was purified by flash chromatography (hexanes/diethyl ether) to yield the title compound (205 mg, 66%, mixture of 2 diastereoisomers) as a colourless oil which solidifies upon standing at room temperature.

MS (m/z): 374 [M+1]⁺

¹H-NMR δ (400 MHz, CDCl₃, only one stereoisomer is described): 0.88 (t, J=7 Hz, 3H), 1.12-1.43 (m, 21H), 1.42-1.68 (m, 2H), 2.18 (d, J=5 Hz, 1H), 4.35 (q, J=7 Hz, 2H), 4.58-4.94 (m, 2H), 6.84 (t, J=4 Hz, 1H), 8.70 (s, 1H).

### c) Ethyl 3-fluoro-4-(2-fluorotridecyl)-1H-pyrrole-2-carboxylate

To a solution of ethyl 3-fluoro-4-(2-fluoro-1-hydroxytridecyl)-1H-pyrrole-2-carboxylate (Intermediate 41b, 205 mg, 0.55 mmol) in DCM (5 mL) were added TFA (0.21 mL, 2.75 mmol) and triethylsilane (0.26 mL, 1.65 mmol) and the resulting mixture was stirred at room temperature for 3 h. The reaction mixture was diluted with DCM, washed with saturated aqueous sodium hydrogen carbonate solution (×2) and brine, dried over magnesium sulfate, filtered and the solvent was evaporated. The residue was purified by flash chromatography (hexanes/diethyl ether) to yield the title compound (44 mg, 22%) as a white solid.

MS (m/z): 358 [M+1]⁺

¹H-NMR δ (400 MHz, CDCl₃): 0.87 (m, 3H), 1.42-1.20 (m, 21H), 1.72-1.51 (m, 2H), 2.73 (dd, J=23 and 7 Hz, 2H), 4.34 (q, J=7 Hz, 2H), 4.61 (dd, J=53 and 10 Hz, 1H), 6.77-6.61 (m, 1H), 8.62 (s, 1H).

### Intermediate 42

### Ethyl 4-(2,2-difluorotridecanoyl)-3-fluoro-1H-pyrrole-2-carboxylate

To a cooled (−78° C.) solution of diisopropylamine (0.29 mL, 2.06 mmol) in THF (3 mL) was added dropwise n-butyl lithium (1.6 M solution in hexanes, 1.29 mL, 2.06 mmol) and the resulting mixture was stirred at −78° C. for 30 minutes. A solution of ethyl 3-fluoro-4-(2-fluorotridecanoyl)-1H-pyrrole-2-carboxylate (Intermediate 42a, 255 mg, 0.69 mmol) in THF (3 mL) was added dropwise and the mixture was stirred for 1 h at −40° C. After cooling again to −78° C., a solution of N-fluorobenzenesulfonimide (520 mg, 1.65 mmol) in THF (2 mL) was added dropwise and the mixture was stirred at −78° C. for 2 h and then allowed to warm to room temperature. After stirring for 2 h at room temperature, water was slowly added and the reaction mixture was acidified to pH=2-3 by addition of 1N hydrochloric acid solution. The reaction mixture was extracted with EtOAc (×3) and the combined organic extracts were washed with brine, dried over magnesium sulfate, filtered and the solvent was evaporated. The residue was purified by flash chromatography (hexanes/diethyl ether) to yield the title compound (177 mg, 66%) as a slightly yellow solid.

MS (m/z): 390 [M+1]⁺

¹H-NMR δ (400 MHz, CDCl₃): 0.96-0.79 (m, 3H), 1.44-1.18 (m, 19H), 1.56-1.47 (m, 2H), 2.10 (q, J=16 and 14 Hz, 2H), 4.39 (q, J=7 Hz, 2H), 7.53 (d, J=2 Hz, 1H), 9.19 (brs, 1H).

### Intermediate 43

### Ethyl 4-(3,3-dimethyldodecyl)-3-fluoro-1H-pyrrole-2-carboxylate

### a) 3,3-Dimethyldodecanoic acid

To a solution of copper(I) iodide (167 mg, 0.87 mmol), trimethylsilyl chloride (1.3 mL, 10.24 mmol) and methyl 3-methylbut-2-enoate (1.0 mL, 8.23 mmol) in THF (60 mL) at −15° C. was added dropwise bromo(nonyl)magnesium (1M in diethyl ether solution, 10.5 mL, 10.5 mmol) and the resulting mixture was stirred at room temperature overnight. Saturated aqueous ammonium chloride was added to the reaction mixture, volatiles were removed under reduced pressure and the crude was partitioned between hexanes and water. The organic layer was separated, dried over magnesium sulfate and the solvent evaporated to yield methyl 3,3-dimethyldodecanoate (2.0 g, 100%) as a colourless oil which was used in the next synthetic step without further purification.

¹H-NMR δ (400 MHz, CDCl₃): 0.85-0.92 (m, 3H), 0.97 (s, 6H), 1.22-1.32 (m, 16H), 2.19 (s, 2H), 3.64 (s, 3H).

To a solution of methyl 3,3-dimethyldodecanoate (2.0 g, 8.23 mmol) in ethanol (20 mL) was added potassium hydroxide (2.45 g, 41.15 mmol) followed by water (2 mL) and the resulting mixture was heated at reflux for 18 h. After cooling to room temperature, the solvent was removed under reduced pressure and the crude was partitioned between water and hexanes. Phases were separated, the aqueous phase was acidified to pH=2-3 by addition of 1N hydrochloric acid solution and extracted with diethyl ether (×3). The combined organic extracts were washed with brine, dried over magnesium sulfate, filtered and the solvent was evaporated to yield the title product (1.34 g, 71%) as an orange oil.

¹H-NMR δ (400 MHz, CDCl₃): 0.81-0.93 (m, 3H), 1.01 (s, 6H), 1.26 (m, 16H), 2.22 (s, 2H).

### b) 3,3-Dimethyldodecanoyl chloride

To a solution of 3,3-dimethyldodecanoic acid (Intermediate 43a, 600 mg, 2.62 mmol) in DCM (11 mL) was added oxalyl chloride (520 μL, 6.05 mmol) and DMF (1 drop) and the resulting mixture was stirred at room temperature overnight. Solvent was then evaporated to yield the title compound (680 mg, 100%) as an oil which was used in the next synthetic step without further purification.

¹H-NMR δ (400 MHz, CDCl₃): 0.83-0.95 (m, 3H), 1.03 (s, 6H), 1.14-1.40 (m, 16H), 2.83 (s, 2H).

### c) Ethyl 4-(3,3-dimethyldodecanoyl)-3-fluoro-1H-pyrrole-2-carboxylate

To a solution of ethyl 3-fluoro-1H-pyrrole-2-carboxylate (150 mg, 0.95 mmol) in benzene (1.5 mL) under an argon atmosphere was added a solution of 3,3-dimethyldodecanoyl chloride (Intermediate 43b, 470 mg, 1.9 mmol) in benzene (1 mL) followed by tin(IV) tetrachloride (168 μL, 0.37 mmol) and the resulting solution was stirred at room temperature for 1 h 30 min. 1N hydrochloric acid solution (1 mL) was then added and the reaction mixture was extracted with EtOAc (×3). The combined organic extracts were washed with brine, dried over magnesium sulfate, filtered and the solvent was evaporated. The residue was purified by flash chromatography (hexanes/diethyl ether) to yield the title compound (265 mg, 75%) as a colorless oil.

MS (m/z): 368 [M+1]⁺

¹H-NMR δ (400 MHz, CDCl₃): 0.81-0.93 (m, 3H), 1.01 (s, 6H), 1.19-1.30 (m, 16H), 1.38 (t, J=7 Hz, 3H), 2.66 (s, 2H), 4.37 (q, J=7 Hz, 2H), 7.32 (t, J=4 Hz, 1H), 9.05 (s, 1H).

### d) Ethyl 4-(3,3-dimethyldodecyl)-3-fluoro-1H-pyrrole-2-carboxylate

To a solution of ethyl 4-(3,3-dimethyldodecanoyl)-3-fluoro-1H-pyrrole-2-carboxylate (Intermediate 43c, 120 mg, 0.33 mmol) in TFA (2.5 mL) was added dropwise triethylsilane (157 μL, 0.98 mmol) and the resulting mixture was stirred at room temperature overnight. Additional triethylsilane (50 μL) was added and the reaction mixture was stirred at room temperature for further 24 h. The volatiles were removed under reduced pressure, the residue was dissolved in DCM and washed with saturated aqueous solution of sodium hydrogen carbonate and brine. The organic solution was dried over magnesium sulfate, filtered and the solvent was evaporated. The residue was purified by flash chromatography (hexanes/EtOAc) to yield the title product (68 mg, 59%) as a slightly yellow oil.

MS (m/z): 354 [M+1]⁺

¹H-NMR δ (400 MHz, CDCl₃): 0.85-0.92 (m, 9H), 1.19-1.30 (m, 16H), 1.36 (t, J=7 Hz, 3H), 1.40-1.48 (m, 2H), 2.30-2.38 (m, 2H), 4.33 (q, J=7 Hz, 2H), 6.48-6.58 (m, 1H), 8.46 (s, 1H).

### Intermediate 44

### Ethyl 4-((2,2-dimethyltridecyl)oxy)-3-fluoro-1H-pyrrole-2-carboxylate

### a) Ethyl 2,2-dimethyltridecanoate

To a solution of diisopropylamine (2.6 mL, 18.6 mmol) in THF (14 mL) at −78° C. was added n-butyl lithium (2.5 M in hexanes, 7.2 mL, 18.00 mmol). The temperature was allowed to rise to 0° C. and the reaction mixture was stirred at this temperature for 30 min. The solution was cooled to −78° C. and ethyl isobutyrate (2.00 g, 17.13 mmol) was added dropwise. Stirring was continued for 1 h at −78° C. and then 1-bromoundecane (4.17 g, 17.72 mmol) was added. After stirring overnight at room temperature, the reaction mixture was poured into ice/water containing 20 mL of saturated aqueous solution of ammonium chloride. The mixture was then extracted with diethyl ether (×3). The combined organic extracts were washed with brine, dried over anhydrous magnesium sulfate and the solvent was evaporated under reduced pressure to give the title compound (4.65 g, 100%) as a yellow oil which was used in the next synthetic step without further purification.

### b) 2,2-Dimethyltridecanoic acid

To a solution of ethyl 2,2-dimethyltridecanoate (Intermediate 44a, 2.00 g, 7.39 mmol) in ethanol (20 mL) was added a solution of potassium hydroxide (2.06 g, 36.71 mmol) in water (4 mL) and the reaction mixture was stirred at 70° C. overnight. The solvent was evaporated under reduced pressure, water was added and the aqueous solution was washed with diethyl ether (×2). The organic layer was discarded and the aqueous phase was acidified to pH=5 by addition of 5N aqueous hydrochloric acid solution. The aqueous phase was extracted with diethyl ether (×3). The combined organic extracts were washed with brine, dried over magnesium sulfate, filtered and the solvent was evaporated under reduced pressure to give the title compound (1.09 g, 61%) as a yellow semisolid.

¹H-NMR δ (400 MHz, CDCl₃): 0.88 (t, J=6.9 Hz, 3H), 1.19 (s, 6H), 1.26-1.29 (m, 20H).

### c) 2,2-Dimethyltridecan-1-ol

To a suspension of lithium aluminium hydride (313 mg, 8.24 mmol) in THF (5 mL) and under argon atmosphere was added dropwise a solution of 2,2-dimethyltridecanoic acid (Intermediate 44b, 500 mg, 2.06 mmol) in THF (5 mL) and the mixture was heated at 60° C. for 2 h. The reaction was cooled to room temperature and water (0.33 mL), aqueous 4N sodium hydroxide solution (0.33 mL) and water (1 mL) were slowly added in this order. The reaction mixture was diluted with EtOAc and solids were filtered. The organic layer was separated and the aqueous phase was extracted with EtOAc (×2). The combined organic layers were washed with brine, dried over magnesium sulfate and the solvent was removed under reduced pressure to give the title compound (385 mg, 82%) as a colourless oil which was used in the next synthetic step without further purification.

¹H-NMR δ (400 MHz, CDCl₃): 0.86 (s, 6H), 0.88 (t, J=6.9 Hz, 3H), 1.24-1.29 (m, 20H), 3.31 (s, 2H).

### d) 2,2-Dimethyltridecyl trifluoromethanesulfonate

To a solution of 2,2-dimethyltridecan-1-ol (Intermediate 44c, 200 mg, 0.88 mmol) and pyridine (73 μL, 0.96 mmol) in DCM (5 mL) under argon atmosphere at 0° C. was added trifluoromethylsulfonyl trifluoromethanesulfonate (162 μL, 0.96 mmol) and the resulting solution was stirred at room temperature for 45 min. After cooling to 0° C., water was added and the reaction mixture was partitioned between water and DCM. The aqueous layer was separated and washed with DCM (×3). The combined organic layers were filtered through a Phase Separator and the solvent was removed under reduced pressure to give the title compound (307 mg, 97%) as a light yellow oil which was used in the next synthetic step without further purification.

¹H-NMR δ (400 MHz, CDCl₃): 0.88 (t, J=6.8 Hz, 3H), 0.98 (s, 6H), 1.25-1.30 (m, 20H), 4.20 (s, 2H).

### e) Ethyl 4-((2,2-dimethyltridecyl)oxy)-3-fluoro-1H-pyrrole-2-carboxylate

Obtained as a colourless oil (14%) from ethyl 3-fluoro-4-hydroxy-1H-pyrrole-2-carboxylate (Intermediate 28c) and 2,2-dimethyltridecyl trifluoromethane sulfonate (Intermediate 44d) following the experimental procedure described in Intermediate 1d.

¹H-NMR δ (400 MHz, CDCl₃): 0.78-0.86 (m, 3H), 1.07 (s, 6H), 1.17-1.25 (m, 20H), 1.29 (t, J=7.1 Hz, 3H), 3.51 (s, 2H), 4.27 (q, J=7.1 Hz, 2H), 6.32-6.41 (m, 1H).

### Intermediate 45

### Ethyl 4-((2,2-difluorotetradecyl)oxy)-3-fluoro-1H-pyrrole-2-carboxylate

### a) 2,2-Difluorotetradecan-1-ol

To a solution of tetradecanal (500 mg, 2.35 mmol) in THF (20 mL) were added pyrrolidine-2-carboxylic acid (542 mg, 4.71 mmol) and N-fluoro-N-(phenylsulfonyl)benzenesulfonamide (1.85 g, 5.86 mmol) and the mixture was stirred at room temperature for 20 h. Saturated aqueous solution of potassium hydrogencarbonate (20 mL) was then added and the resulting mixture was stirred vigorously for 10 min. The resulting precipitate was filtered, washed with water and the filtrate was extracted with EtOAc (×3). The combined organic layers were washed with saturated aqueous solution of potassium carbonate, dried over magnesium sulfate and the solvent was removed under reduced pressure. The resulting oil was dissolved in a mixture of DCM/methanol (14 mL/9 mL) and sodium borohydride (267 mg, 7.06 mmol) was added. The mixture was stirred at room temperature for 2 h. After cooling to 0° C., saturated aqueous solution of sodium potassium tartrate (10 mL) was added and the mixture was vigorously stirred for 20 min before being extracted with DCM (×3). The combined organic extracts were washed with brine, dried over magnesium sulfate and the solvent was removed under reduced pressure. The residue was purified by flash chromatography (hexanes to diethyl ether) to yield the title compound (310 mg, 53%) as a colourless oil.

¹H-NMR δ (400 MHz, CDCl₃): 0.88 (t, J=6.9 Hz, 3H), 1.23-1.35 (m, 18H), 1.42-1.54 (m, 1H), 1.76-1.99 (m, 3H), 3.67-3.79 (m, 2H).

### b) 2,2-Difluorotetradecyl trifluoromethanesulfonate

Obtained as a yellow oil (100%) from 2,2-difluorotetradecan-1-ol (Intermediate 45a) following the experimental procedure described in Intermediate 44d.

¹H-NMR δ (400 MHz, CDCl₃): 0.87 (t, J=6.9 Hz, 3H), 1.22-1.40 (m, 18H), 1.45-1.55 (m, 2H), 1.80-2.08 (m, 2H), 4.51 (t, J=11.2 Hz, 2H).

### c) Ethyl 4-((2,2-difluorotetradecyl)oxy)-3-fluoro-1H-pyrrole-2-carboxylate

Obtained as a colourless oil (31%) from ethyl 3-fluoro-4-hydroxy-1H-pyrrole-2-carboxylate (Intermediate 28c) and 2,2-difluorotetradecyl trifluoromethane sulfonate (Intermediate 45b) following the experimental procedure described in Intermediate 1d.

¹H-NMR δ (400 MHz, CDCl₃): 0.85-0.91 (m, 3H), 1.22-1.33 (m, 18H), 1.36 (t, J=7.1 Hz, 3H), 1.52 (m, 2H), 1.91-2.08 (m, 2H), 4.08 (t, J=11.9 Hz, 2H), 4.35 (q, J=7.1 Hz, 2H), 6.53 (t, J=4.0 Hz, 1H), 8.24 (s, 1H).

### Intermediate 46

### Ethyl 4-((2,2-difluoroundecyl)oxy)-3-fluoro-1H-pyrrole-2-carboxylate

### a) 2,2-Difluoroundecan-1-ol

Obtained as a white solid (53%) from undecanal following the experimental procedure described in Intermediate 45a.

¹H-NMR δ (400 MHz, CDCl₃): 0.84-0.92 (m, 3H), 1.25-1.36 (m, 12H), 1.44-1.53 (m, 2H), 1.81-1.95 (m, 2H), 3.73 (td, J=12.8, 6.9 Hz, 2H).

### b) 2,2-Difluoroundecyl trifluoromethanesulfonate

Obtained as a light brown solid (99%) from 2,2-difluoroundecan-1-ol (Intermediate 46a) following the experimental procedure described in Intermediate 44d.

¹H-NMR δ (400 MHz, CDCl₃): 0.85-0.91 (m, 3H), 1.23-1.39 (m, 12H), 1.46-1.54 (m, 2H), 1.86-2.04 (m, 2H), 4.51 (t, J=11.2 Hz, 2H).

### c) Ethyl 4-((2,2-difluoroundecyl)oxy)-3-fluoro-1H-pyrrole-2-carboxylate

Obtained as a beige solid (30%) from ethyl 3-fluoro-4-hydroxy-1H-pyrrole-2-carboxylate (Intermediate 28c) and 2,2-difluoroundecyl trifluoromethane sulfonate (Intermediate 46b) following the experimental procedure described in Intermediate 1d.

¹H-NMR δ (400 MHz, CDCl₃): 0.85-0.91 (m, 3H), 1.25-1.33 (m, 12H), 1.36 (t, J=7.1 Hz, 3H), 1.51 (m, 2H), 1.92-2.09 (m, 2H), 4.08 (t, J=11.9 Hz, 2H), 4.35 (q, J=7.1 Hz, 2H), 6.53 (t, J=4.0 Hz, 1H), 8.23 (s, 1H).

### Intermediate 47

### Methyl 3-chloro-4-((2-fluorotetradecyl)oxy)-1H-pyrrole-2-carboxylate

### a) 1-Bromo-2-fluorotetradecane

To a solution of tetradec-1-ene (5.00 g, 25.45 mmol) in DCM (55 mL) was added a solution of triethylamine hydrofluoride (12.31 g, 76.36 mmol) in DCM (5 mL) and the resulting solution was cooled to 0° C. and protected from the light. N-bromosuccinimide (4.98 g, 27.98 mmol) was then added in portions and the reaction mixture was stirred at room temperature for 6 h, poured into a mixture of ice/water and extracted with DCM (×3). The combined organic layers were washed with 0.5N hydrochloric acid solution, 4% aqueous sodium hydrogen carbonate solution and brine, dried over magnesium sulfate and the solvent was evaporated under reduced pressure to give the title compound (7.50 g, 100%) as a colourless oil.

¹H-NMR δ (400 MHz, CDCl₃): 0.84-0.92 (m, 3H), 1.23-1.29 (m, 18H), 1.36-1.52 (m, 2H), 1.69-1.81 (m, 2H), 3.48 (m, 2H), 4.50-4.78 (m, 1H).

### b) Methyl 3-chloro-4-((2-fluorotetradecyl)oxy)-1H-pyrrole-2-carboxylate

Obtained (11%) from methyl 3-chloro-4-hydroxy-1H-pyrrole-2-carboxylate (Intermediate 34c) and 1-bromo-2-fluorotetradecane (Intermediate 47a) following the experimental procedure described in Intermediate 1d followed by purification of the crude product by flash chromatography (hexanes/DCM).

¹H NMR δ (400 MHz, CDCl₃) 0.83-0.92 (m, 3H), 1.26 (s, 20H), 1.58-1.84 (m, 2H), 3.89 (s, 3H), 3.97-4.03 (m, 1H), 4.03-4.08 (m, 1H), 4.64-4.98 (m, 1H), 6.59 (d, J=3.5 Hz, 1H), 8.62 (s, 1H).

### Intermediate 48

### Ethyl 3-chloro-4-((9-ethoxynonyl)oxy)-1H-pyrrole-2-carboxylate

### a) Methyl 3-chloro-4-((9-hydroxynonyl)oxy)-1H-pyrrole-2-carboxylate

A mixture of methyl 3-chloro-4-hydroxy-1H-pyrrole-2-carboxylate (Intermediate 34c, 250 mg, 1.42 mmol), 9-bromononan-1-ol (318 mg, 1.42 mmol) and potassium carbonate (394 mg, 2.85 mmol) in DMF (4 mL) was heated at 100° C. for 16 h. After cooling to room temperature, 1M Hydrochloric acid solution was added until an acidic pH was reached and the reaction mixture was extracted with EtOAc (×3). The combined organic extracts were washed with brine, dried over magnesium sulfate, filtered and the solvent was evaporated to dryness. The resulting crude was purified by flash chromatography (hexanes/diethyl ether) to yield the title compound (180 mg, 37%) as a colourless oil.

MS (m/z): 318 [M+1]⁺

¹H NMR δ (400 MHz, CDCl₃) 0.25-1.51 (m, 12H), 1.51-1.66 (m, 2H), 1.69-1.88 (m, 2H), 3.57-3.73 (m, 2H), 3.82-4.01 (m, 5H), 6.52 (d, J=3.5 Hz, 1H), 8.61 (s, 1H).

### b) Methyl 3-chloro-4-((9-((methylsulfonyl)oxy)nonyl)oxy)-1H-pyrrole-2-carboxylate

To a cooled (0° C.) solution of methyl 3-chloro-4-((9-hydroxynonyl)oxy)-1H-pyrrole-2-carboxylate (Intermediate 48a, 180 mg, 0.56 mmol) in pyridine (3 mL) was added methanesulfonyl chloride (48 μL, 0.62 mmol) and the resulting mixture was stirred at 0° C. for 2 h 30 min. Ice and water were added and the reaction mixture was extracted with diethyl ether (×3). The combined organic phases were washed with 6M hydrochloric acid solution, dried over magnesium sulfate, filtered and the solvent was evaporated. The crude was purified by flash chromatography (hexanes/DCM) to yield the title compound (75 mg, 33%).

MS (m/z): 396 [M+1]⁺

¹H NMR δ (400 MHz, CDCl₃) 1.29-1.49 (m, 10H), 1.69-1.82 (m, 4H), 3.00 (s, 3H), 3.84-3.97 (m, 5H), 4.22 (t, J=6.6 Hz, 2H), 6.52 (d, J=3.5 Hz, 1H), 8.64 (s, 1H).

### c) Ethyl 3-chloro-4-((9-ethoxynonyl)oxy)-1H-pyrrole-2-carboxylate

To a solution of sodium (23 mg, 5.06 mmol) in ethanol (3 mL) was added methyl 3-chloro-4-(9-methylsulfonyloxynonoxy)-1H-pyrrole-2-carboxylate (Intermediate 48b, 75 mg, 0.19 mmol) and the resulting mixture was heated at 70° C. for 4 h. The solvent was evaporated and the residue was partitioned between water and DCM. The organic layer was separated and the aqueous layer was extracted with DCM (×3). The combined organic extracts were dried over magnesium sulfate, filtered and the solvent was evaporated to dryness. The crude was purified by flash chromatography (DCM/methanol) to give the title compound (33 mg, 48%) as an oil.

MS (m/z): 360 [M+1]⁺

¹H NMR δ (400 MHz, CDCl₃) 1.19 (t, J=7.0 Hz, 3H), 1.27-1.49 (m, 13H), 1.50-1.62 (m, 2H), 1.70-1.82 (m, 2H), 3.40 (t, J=6.8 Hz, 2H), 3.46 (q, J=7.0 Hz, 2H), 3.89 (t, J=6.6 Hz, 2H), 4.35 (q, J=7.1 Hz, 2H), 6.51 (d, J=3.5 Hz, 1H), 8.73 (s, 1H).

### Intermediate 49

### Ethyl 3-methyl-4-tridecyl-1H-pyrrole-2-carboxylate

### a) Ethyl 3-methyl-4-tridecanoyl-1H-pyrrole-2-carboxylate

Obtained (67%) from ethyl 3-methyl-1H-pyrrole-2-carboxylate and tridecanoyl chloride (Intermediate 8a) following the experimental procedure described in Intermediate 3b followed by purification of the crude product by flash chromatography (hexanes/EtOAc).

MS (m/z): 350 [M+1]⁺

¹H NMR δ (400 MHz, CDCl₃): 0.81-0.94 (m, 3H), 1.16-1.43 (m, 21H), 1.62-1.76 (m, 2H), 2.63 (s, 3H), 2.71 (m, 3H), 4.35 (q, J=7 Hz, 2H), 7.44 (d, J=3 Hz, 1H).

### b) Ethyl 3-methyl-4-tridecyl-1H-pyrrole-2-carboxylate

Obtained (50%) from ethyl 3-methyl-4-tridecanoyl-1H-pyrrole-2-carboxylate (Intermediate 49a) following the experimental procedure described in Intermediate 3c followed by purification of the crude product by flash chromatography (hexanes/EtOAc).

MS (m/z): 336 [M+1]⁺

¹H NMR δ (400 MHz, CDCl₃): 0.84-0.91 (m, 3H), 1.19-1.39 (m, 23H), 1.45-1.54 (m, 2H), 2.28 (s, 3H), 2.34-2.42 (m, 2H), 4.30 (q, J=7 Hz, 2H), 6.65 (d, J=3 Hz, 1H), 8.70 (s, 1H, bb).

### Intermediate 50

### Ethyl 4-(2,2-dimethyldodecyl)-3-fluoro-1H-pyrrole-2-carboxylate

### a) 2,2-Dimethyldodecanoyl chloride

Obtained (93%) from 2,2-dimethyldodecanoic acid and oxalyl chloride following the experimental procedure described in Intermediate 3a.

¹H NMR δ (400 MHz, CDCl₃): 0.85-0.91 (m, 3H), 1.24-1.32 (m, 22H), 1.59-1.66 (m, 2H).

### b) Ethyl 4-(2,2-dimethyldodecanoyl)-3-fluoro-1H-pyrrole-2-carboxylate

To a cooled (0° C.) solution of ethyl 3-fluoro-1H-pyrrole-2-carboxylate (20 mg, 0.012 mmol) in 1,2-dichloroethane (1 mL) were added boron trifluoride diethyl etherate (31 μL, 0.25 mmol) and 2,2-dimethyldodecanoyl chloride (Intermediate 50a, 63 mg, 0.25 mmol) and the resulting mixture was stirred at ambient temperature for 6 days. The reaction mixture was partitioned between water and DCM, the organic layer was separated and the aqueous layer was washed with DCM (×2). The combined organic phases were dried over magnesium sulfate, filtered and the solvent was evaporated to dryness. The resulting crude was purified by flash chromatography (hexanes/DCM) to yield the title compound (18 mg, 38%) as a solid.

¹H NMR δ (400 MHz, CDCl₃): 0.87 (t, J=6.9 Hz, 3H), 1.19-1.32 (m, 22H), 1.39 (t, J=7.1 Hz, 3H), 1.64-1.72 (m, 2H), 4.38 (q, J=7.1 Hz, 2H), 7.37 (t, J=4.0 Hz, 1H).

### c) Ethyl 4-(2,2-dimethyldodecyl)-3-fluoro-1H-pyrrole-2-carboxylate

Obtained (69%) from ethyl 4-(2,2-dimethyldodecanoyl)-3-fluoro-1H-pyrrole-2-carboxylate (Intermediate 50b) following the experimental procedure described in Intermediate 3c followed by purification of the crude product by flash chromatography (hexanes/EtOAc).

MS (m/z): 354 [M+1]⁺

¹H NMR δ (400 MHz, CDCl₃): 0.84 (s, 6H), 0.85-0.92 (m, 3H), 1.22-1.32 (m, 18H), 1.36 (t, J=7.1 Hz, 3H), 2.29 (s, 2H), 4.34 (q, J=7.1 Hz, 2H), 6.48-6.56 (m, 1H), 8.63 (s, 1H).

### Intermediate 51

### Ethyl 3-fluoro-5-undecyl-1H-pyrrole-2-carboxylate

To a solution of ethyl 3-fluoro-1H-pyrrole-2-carboxylate (75 mg, 0.47 mmol) in DMA (0.5 mL) were added potassium hydrogenphosphate (266 mg, 1.52 mmol), norbornene (90 mg, 0.95 mmol), dichlorobis(acetonitrile)palladium (II) (12 mg, 0.046 mmol) and 1-bromoundecane (0.22 mL, 1.00 mmol). The resulting mixture was heated at 90° C. under an air atmosphere in a Kimax reactor for 21 h. After cooling to room temperature, the reaction mixture was diluted with diethyl ether and filtered through a pad of Celite®. The filtrate was washed with water and brine, dried over magnesium sulfate and the solvent was evaporated to dryness. The residue was purified by flash chromatography (hexanes/DCM) to yield the title compound (112 mg, 75%) as a white solid.

MS (m/z): 312 [M+1]⁺

¹H NMR δ (400 MHz, CDCl₃): 0.85-0.92 (m, 3H), 1.20-1.33 (m, 16H), 1.35 (t, J=7.1 Hz, 2H), 1.55-1.70 (m, 2H), 2.47-2.57 (m, 2H), 4.32 (q, J=7.1 Hz, 2H), 5.73 (d, J=3.2 Hz, 1H), 8.26 (s, 1H).

### Intermediate 52

### Ethyl 3-fluoro-5-tridecyl-1H-pyrrole-2-carboxylate

### a) Ethyl 3-fluoro-5-tridecanoyl-1H-pyrrole-2-carboxylate

To a cooled (0° C.) solution of tridecanoyl chloride (Intermediate 8a, 741 mg, 3.18 mmol) in dichloroethane (3 mL) were added zinc(II) chloride (433 mg, 3.17 mmol) and a solution of ethyl 3-fluoro-1H-pyrrole-2-carboxylate (250 mg, 1.59 mmol) in dichloroethane (2 mL) and the resulting mixture was stirred at 50° C. for 1 h 30 min. The reaction mixture was cooled down to room temperature, poured into ice-water and extracted with EtOAc (×2). The combined organic extracts were washed with saturated aqueous solution of sodium hydrogen carbonate and brine, dried over magnesium sulfate, filtered and the solvent was evaporated. The residue was purified by flash chromatography (hexanes/DCM) to give the title compound (99 mg, 18%) as a solid.

¹H NMR δ (400 MHz, CDCl₃): 0.81-0.96 (m, 3H), 1.19-1.43 (m, 12H), 1.58-1.77 (m, 2H), 2.32-2.40 (m, 2H), 4.38 (q, J=7.1 Hz, 2H), 6.53 (dd, J=3.1, 0.9 Hz, 1H).

### b) Ethyl 3-fluoro-5-tridecyl-1H-pyrrole-2-carboxylate

Obtained (29%) from ethyl 3-fluoro-5-tridecanoyl-1H-pyrrole-2-carboxylate (Intermediate 52a) following the experimental procedure described in Intermediate 3c followed by purification of the crude product by flash chromatography (hexanes/DCM).

MS (m/z): 340 [M+1]⁺

¹H NMR δ (400 MHz, CDCl₃): 0.77-0.99 (m, 3H), 1.15-1.43 (m, 23H), 1.52-1.78 (m, 2H), 2.53 (t, J=7.7 Hz, 2H), 4.33 (q, J=7.1 Hz, 2H), 5.72 (d, J=3.2 Hz, 1H), 8.59 (s, 1H).

### Intermediate 53

### Ethyl 3-fluoro-5-tetradecyl-1H-pyrrole-2-carboxylate

Obtained as a white solid (60%) from ethyl 3-fluoro-1H-pyrrole-2-carboxylate and 1-bromotetradecane following the experimental procedure described in Intermediate 51 followed by purification of the crude product by flash chromatography (hexanes/EtOAc).

MS (m/z): 354 [M+1]⁺.

¹H-NMR δ (400 MHz, CDCl₃): 0.88 (t, J=7 Hz, 3H), 1.21-1.33 (m, 22H), 1.35 (t, J=7 Hz, 3H), 1.53-1.63 (m, 2H), 2.52 (t, J=8 Hz, 2H), 4.32 (q, J=7 Hz, 2H), 5.73 (d, J=3 Hz, 1H), 8.30 (brs, 1H).

### Intermediate 54

### Ethyl 3-fluoro-5-pentadecyl-1H-pyrrole-2-carboxylate

Obtained as a white solid (80%) from ethyl 3-fluoro-1H-pyrrole-2-carboxylate and 1-bromopentadecane following the experimental procedure described in Intermediate 51 followed by purification of the crude product by flash chromatography (hexanes/EtOAc).

MS (m/z): 368 [M+1]⁺.

¹H-NMR δ (400 MHz, CDCl₃): 0.88 (t, J=7 Hz, 3H), 1.20-1.33 (m, 24H), 1.35 (t, J=7 Hz, 3H), 1.59-1.63 (m, 2H), 2.52 (t, J=8 Hz, 2H), 4.32 (q, J=7 Hz, 2H), 5.73 (d, J=3 Hz, 1H), 8.33 (brs, 1H).

### Intermediate 55

### Ethyl 3-fluoro-5-hexadecyl-1H-pyrrole-2-carboxylate

Obtained (33%) from ethyl 3-fluoro-1H-pyrrole-2-carboxylate and 1-bromohexadecane following the experimental procedure described in Intermediate 51 followed by purification of the crude product by flash chromatography (hexanes/EtOAc).

MS (m/z): 382 [M+1]⁺.

### Intermediate 56

### Ethyl 3-fluoro-5-heptadecyl-1H-pyrrole-2-carboxylate

Obtained (33%) from ethyl 3-fluoro-1H-pyrrole-2-carboxylate and 1-bromoheptadecane following the experimental procedure described in Intermediate 51 followed by purification of the crude product by flash chromatography (hexanes/diethyl ether).

MS (m/z): 396 [M+1]⁺.

¹H NMR δ (400 MHz, CDCl₃): 0.85-0.91 (m, 3H), 1.22-1.33 (m, 28H), 1.36 (t, J=7 Hz, 3H), 1.56-1.65 (m, 2H), 2.56 (t, J=8.2 Hz, 2H), 4.32 (q, J=7.1 Hz, 2H), 5.73 (d, J=3.2 Hz, 1H), 8.29 (s, 1H).

### Intermediate 57

### Ethyl 3-fluoro-5-octadecyl-1H-pyrrole-2-carboxylate

Obtained (63%) from ethyl 3-fluoro-1H-pyrrole-2-carboxylate and 1-bromooctadecane following the experimental procedure described in Intermediate 51 followed by purification of the crude product by flash chromatography (hexanes/diethyl ether).

MS (m/z): 410 [M+1]⁺.

¹H NMR δ (400 MHz, CDCl₃): 0.85-0.91 (m, 3H), 1.22-1.33 (m, 30H), 1.36 (t, J=7 Hz, 3H), 1.56-1.65 (m, 2H), 2.56 (t, J=8.2 Hz, 2H), 4.32 (q, J=7.1 Hz, 2H), 5.73 (d, J=3.2 Hz, 1H), 8.29 (s, 1H).

### Intermediate 58

### Ethyl 3-fluoro-5-nonadecyl-1H-pyrrole-2-carboxylate

Obtained (52%) from ethyl 3-fluoro-1H-pyrrole-2-carboxylate and 1-bromononadecane following the experimental procedure described in Intermediate 51 followed by purification of the crude product by flash chromatography (hexanes/diethyl ether).

MS (m/z): 424 [M+1]⁺.

¹H NMR δ (400 MHz, DMSO-d6): 0.81-0.89 (m, 3H), 1.20-1.27 (m, 32H), 1.36 (t, J=7 Hz, 3H), 1.45-1.59 (m, 2H), 2.48 (t, J=8.2 Hz, 2H), 4.21 (q, J=7.1 Hz, 2H), 5.80 (d, J=3.2 Hz, 1H), 11.37 (s, 1H).

### Intermediate 59

### Methyl 3-chloro-5-(2,2-dimethyldodecyl)-1H-pyrrole-2-carboxylate

### a) Methyl 3-chloro-5-(2,2-dimethyldodecanoyl)-1H-pyrrole-2-carboxylate

Obtained (46%) from methyl 3-chloro-1H-pyrrole-2-carboxylate and 2,2-dimethyldodecanoyl chloride (Intermediate 50a) following the experimental procedure described in Intermediate 23a heating the reaction mixture at 50° C. for 17 h. The crude product was purified by flash chromatography (hexanes/diethyl ether).

¹H NMR δ (400 MHz, CDCl₃): 0.83-0.91 (m, 3H), 1.16-1.34 (m, 22H), 1.68-1.76 (m, 2H), 3.92 (s, 3H), 6.83 (d, J=3.0 Hz, 1H), 9.81 (s, 1H).

### b) Methyl 3-chloro-5-(2,2-dimethyldodecyl)-1H-pyrrole-2-carboxylate

Obtained (51%) from methyl 3-chloro-5-(2,2-dimethyldodecanoyl)-1H-pyrrole-2-carboxylate (Intermediate 59a) following the experimental procedure described in Intermediate 3c followed by purification of the crude product by flash chromatography (hexanes/DCM).

¹H NMR δ (400 MHz, CDCl₃): 0.83-0.93 (m, 246H), 1.23-1.31 (m, 18H), 2.40 (s, 2H), 3.87 (s, 3H), 5.96 (d, J=3.1 Hz, 1H), 8.65 (s, 1H).

### Intermediate 60

### Methyl 3-chloro-5-(3,3-difluorododecyl)-1H-pyrrole-2-carboxylate

### a) 3,3-Difluorododecan-1-ol

To a solution of 3,3-difluorododecanoic acid (prepared as described in WO9965889, 236 mg, 1 mmol) in THF (7 mL) was added dropwise a solution of lithium aluminium hydride in THF (1M, 4 mL, 4 mmol) and the resulting mixture was heated at 70° C. for 18 h. After cooling to room temperature, 1N aqueous sodium hydroxide solution was added, the reaction mixture was stirred for 15 minutes and the solid formed was filtered. Diethyl ether was added to the filtrate and phases were separated. The organic phase was dried over magnesium sulfate, filtered and solvent removed under reduced pressure. The residue was purified by flash chromatography (hexanes/EtOAc) to give the title compound (30 mg, 13%) as a grey oil.

¹H-NMR δ (400 MHz, CDCl₃): 0.88 (t, J=7 Hz, 3H), 1.20-1.37 (m, 12H), 1.47 (p, J=8 Hz, 2H), 1.79-1.92 (m, 2H), 2.06-2.18 (m, 2H), 3.87 (t, J=6 Hz, 2H).

### b) 1-Bromo-3,3-difluoro-dodecane

To a cooled (0° C.) solution of 3,3-difluorododecan-1-ol (Intermediate 60a, 30 mg, 0.135 mmol) in DCM (2 mL) were added triphenylphosphine (46 mg, 0.175 mmol) and NBS (31 mg, 0.174 mmol) and the resulting mixture was stirred at room temperature for 3 h. Water was then added and phases were separated. The organic phase was dried over magnesium sulfate, filtered and solvent removed under reduced pressure. The residue was purified by flash chromatography (hexanes/EtOAc) to yield the title compound (27 mg, 70%) as a grey oil.

¹H-NMR δ (400 MHz, CDCl₃): 0.88 (t, J=7 Hz, 3H), 1.20-1.37 (m, 12H), 1.41-1.51 (m, 2H), 1.75-1.89 (m, 2H), 2.34-2.49 (m, 2H), 3.44-3.48 (m, 2H).

### c) Methyl 3-chloro-5-(3,3-difluorododecyl)-1H-pyrrole-2-carboxylate

Obtained as a white solid (66%) from 1-bromo-3,3-difluoro-dodecane (Intermediate 60b) and methyl 3-chloro-1H-pyrrole-2-carboxylate following the experimental procedure described in Intermediate 51 followed by purification of the crude product by flash chromatography (hexanes/EtOAc).

MS (m/z): 364/366 [M+1/M+3]⁺.

¹H-NMR δ (400 MHz, CDCl₃): 0.88 (t, J=7 Hz, 3H), 1.21-1.35 (m, 12H), 1.40-1.50 (m, 2H), 1.76-1.89 (m, 2H), 2.00-2.18 (m, 2H), 2.76-2.81 (m, 2H), 3.88 (s, 3H), 6.00 (d, J=3 Hz, 1H), 8.92 (brs, 1H).

### Intermediate 61

### Ethyl 3-cyano-5-dodecyl-1H-pyrrole-2-carboxylate

Obtained as a white solid (19%) from ethyl 3-cyano-1H-pyrrole-2-carboxylate and 1-bromododecane following the experimental procedure described in Intermediate 51 followed by purification of the crude product by flash chromatography (hexanes/diethyl ether).

MS (m/z): 333 [M+1]⁺

¹H-NMR δ (400 MHz, CDCl₃): 0.88 (t, J=7 Hz, 3H), 1.22-1.33 (m, 18H), 1.42 (t, J=7 Hz, 3H), 1.59-1.68 (m, 2H), 2.61 (t, J=8 Hz, 2H), 4.40 (q, J=7 Hz, 2H), 6.28 (d, J=3 Hz, 1H), 9.62 (s, 1H).

### Intermediate 62

### Methyl 3-chloro-5-dodecyl-1-methyl-1H-pyrrole-2-carboxylate

### a) Methyl 3-chloro-5-dodecyl-1H-pyrrole-2-carboxylate

Obtained as a white solid (67%) from methyl 3-chloro-1H-pyrrole-2-carboxylate and 1-bromododecane following the experimental procedure described in Intermediate 51 followed by purification of the crude product by flash chromatography (hexanes/EtOAc).

¹H NMR δ (400 MHz, CDCl₃): 0.82-0.93 (m, 3H), 1.19-1.35 (m, 18H), 1.59 (q, J=7.1 Hz, 2H), 2.55 (t, J=7.7 Hz, 2H), 3.87 (s, 3H), 5.97 (d, J=3.1 Hz, 1H), 8.78 (s, 1H).

### b) Methyl 3-chloro-5-dodecyl-1-methyl-1H-pyrrole-2-carboxylate

To a suspension of sodium hydride (60% dispersion in paraffin oil, 15 mg, 0.35 mmol) in DMF (1.5 mL) at 0° C. was added methyl 3-chloro-5-dodecyl-1H-pyrrole-2-carboxylate (Intermediate 62a, 100 mg, 0.30 mmol) and the resulting solution was stirred at 0° C. for 30 min. Iodomethane (38 μL, 0.60 mmol) was added and the solution was stirred at room temperature for 18 h. The reaction mixture was poured into water and extracted with EtOAc (×3). The combined organic extracts were washed with water (×3) and brine, dried over magnesium sulfate and the solvent was removed under reduced pressure. The crude product was purified by flash chromatography to yield the title compound (53 mg, 52%) as a colourless oil.

MS (m/z): 342 [M+1]⁺.

¹H NMR δ (400 MHz, CDCl₃): 0.85-0.91 (m, 3H), 1.25-1.28 (m, 18H), 1.57-1.62 (m, 2H), 2.48-2.55 (m, 2H), 3.77 (s, 3H), 3.85 (s, 3H), 5.94 (s, 1H).

### Intermediate 63

### Ethyl 3-fluoro-5-(14-fluorotetradecyl)-1H-pyrrole-2-carboxylate

### a) 1-Bromo-14-fluorotetradecane

A mixture of 14-bromotetradecan-1-ol (700 mg, 2.38 mmol) and DAST (0.63 mL, 4.76 mmol) was heated at 35° C. for 4 h. The reaction mixture was poured into water and extracted with DCM (×3).

The combined organic extracts were washed with brine, dried over magnesium sulfate, filtered and the solvent was removed under reduced pressure. The residue was purified by flash chromatography (hexanes/diethyl ether) to yield the title compound (422 mg, 60%) as a colourless oil.

¹H NMR δ (400 MHz, CDCl₃): 1.21-1.34 (m, 16H), 1.35-1.47 (m, 4H), 1.60-1.78 (m, 2H), 1.80-1.92 (m, 2H), 3.41 (t, J=6.9 Hz, 2H), 4.38 (t, J=6.2 Hz, 1H), 4.50 (t, J=6.2 Hz, 1H).

### b) Ethyl 3-fluoro-5-(14-fluorotetradecyl)-1H-pyrrole-2-carboxylate

Obtained as a beige solid (18%) from 1-bromo-14-fluorotetradecane (Intermediate 63a) and ethyl 3-fluoro-1H-pyrrole-2-carboxylate following the experimental procedure described in Intermediate 51 followed by purification of the crude product by flash chromatography (hexanes/diethyl ether).

MS (m/z): 372 [M+1]⁺.

¹H NMR δ (600 MHz, CDCl₃): 1.22-1.32 (m, 20H), 1.34 (t, J=6.9 Hz, 3H), 1.36-1.42 (m, 2H), 1.55-1.61 (m, 2H), 1.62-1.73 (m, 2H), 2.51 (t, J=7.1 Hz, 2H), 4.31 (q, J=7.1 Hz, 2H), 4.39 (t, J=6.5 Hz, 1H), 4.46 (t, J=6.5 Hz, 1H), 5.72 (d, J=3.2 Hz, 1H), 8.22 (s, 1H).

### Intermediate 64

### Ethyl 3-fluoro-4-hexadecyl-1H-pyrrole-2-carboxylate

### a) Ethyl 3-fluoro-4-palmitoyl-1H-pyrrole-2-carboxylate

Obtained as a white solid (58%) from ethyl 3-fluoro-1H-pyrrole-2-carboxylate and hexadecanoyl chloride following the experimental procedure described in Intermediate 3b followed by purification by flash chromatography (hexanes to diethyl ether).

MS (m/z): 396 [M+1]⁺.

¹H-NMR δ (400 MHz, DMSO-d₆): 0.88-0.83 (m, 3H), 1.31-1.22 (m, 29H), 1.59-1.49 (m, 2H), 2.70 (t, J=7.3 Hz, 2H), 4.27 (q, J=7.1 Hz, 2H), 7.56 (d, J=4.3 Hz, 1H), 12.42 (s, 1H).

### b) Ethyl 3-fluoro-4-hexadecyl-1H-pyrrole-2-carboxylate

Obtained as a white solid (60%) from ethyl 3-fluoro-4-palmitoyl-1H-pyrrole-2-carboxylate (Intermediate 64a) following the experimental procedure described in Intermediate 3c followed by purification by flash chromatography (hexanes to diethyl ether).

MS (m/z): 380 [M−1]⁺.

¹H-NMR δ (400 MHz, DMSO-d₆): 0.89-0.81 (m, 3H), 1.29-1.21 (m, 29H), 1.51-1.43 (m, 2H), 2.32 (t, J=7.5 Hz, 2H), 4.21 (q, J=7.1 Hz, 2H), 6.71 (d, J=4.7 Hz, 1H), 11.41 (s, 1H).

### Example 1

### 4-(Dodecyloxy)-1H-pyrrole-2-carboxylic acid

To a solution of methyl 4-(dodecyloxy)-1H-pyrrole-2-carboxylate (Intermediate 1 d, 76 mg, 0.24 mmol) in ethanol (2 mL) was added aqueous 1M sodium hydroxide solution (0.98 mL, 0.98 mmol) and the resulting mixture was heated at 85° C. for 21 h. After cooling to room temperature the organic solvent was evaporated. The resulting aqueous residue was acidified to acidic pH by addition of 1M hydrochloric acid solution and extracted with EtOAc (×2). The combined organic extracts were washed with brine, dried over magnesium sulfate, filtered and the solvent evaporated to dryness to yield the title compound (59 mg, 98%) as a solid.

MS (m/z): 296 [M+1]⁺

¹H NMR δ (400 MHz, CDCl₃): 0.86 (t, J=6 Hz, 3H), 1.08-1.48 (m, 18H), 1.58-1.80 (m, 2H), 3.84 (t, J=6 Hz, 2H), 6.56 (s, 1H), 6.53 (s, 1H).

### Example 2

### Ethyl 4-(dodecyloxy)-1H-pyrrole-2-carboxylate

A mixture of 4-(dodecyloxy)-1H-pyrrole-2-carboxylic acid (Example 1.75 mg, 0.25 mmol), ethanol (2 mL), EDC.HCl (58 mg, 0.30 mmol) and 4-DMAP (78 mg, 0.63 mmol) in DCM (2 mL) was stirred at room temperature for 1 h. The mixture was then partitioned between water and DCM and the aqueous layer was separated and washed with DCM (×3). The combined organic phases were dried over magnesium sulfate, filtered and the solvent was evaporated to dryness. The residue was purified by flash chromatography (hexanes/EtOAc) to yield the title compound (39 mg, 47%).

MS (m/z): 324 [M+1]⁺

¹H NMR δ (400 MHz, CDCl₃): 0.88 (t, J=6 Hz, 3H), 1.13-1.54 (m, 21H), 1.62-1.85 (m, 2H), 3.86 (t, J=6 Hz, 2H), 4.30 (q, J=7 Hz, 2H), 6.54 (d, J=3 Hz, 2H), 8.68 (s, 1H).

### Example 3

### 2-(2,5-Dioxopyrrolidin-1-yl)ethyl 4-(dodecyloxy)-1H-pyrrole-2-carboxylate

A mixture of 4-(dodecyloxy)-1H-pyrrole-2-carboxylic acid (Example 1, 80 mg, 0.27 mmol), 1-(2-hydroxyethyl)pyrrolidine-2,5-dione (46 mg, 0.32 mmol), EDC.HCl (62 mg, 0.32 mmol) and 4-DMAP (40 mg, 0.32 mmol) in DCM (1 mL) was stirred at room temperature for 21 h. The mixture was then partitioned between water and DCM and the aqueous layer was separated and washed with DCM (×3). The combined organic phases were dried over magnesium sulfate, filtered and the solvent was evaporated to dryness. The residue was purified by flash chromatography (hexanes/EtOAc) to yield the title compound (61 mg, 53%).

MS (m/z): 421 [M+1]⁺

¹H NMR δ (400 MHz, CDCl₃): 0.88 (t, J=6 Hz, 3H), 1.13-1.47 (m, 18H), 1.62-1.85 (m, 2H), 2.73 (s, 4H), 3.85 (t, 2H), 3.90 (t, 2H), 4.26-4.48 (m, 2H), 6.42-6.64 (m, 2H), 8.71 (s, 1H).

### Example 4

### 2-(2-Oxopyrrolidin-1-yl)ethyl 4-(dodecyloxy)-1H-pyrrole-2-carboxylate

Obtained (44%) from 4-(dodecyloxy)-1H-pyrrole-2-carboxylic acid (Example 1) and 1-(2-hydroxyethyl)pyrrolidin-2-one following the experimental procedure as described in Example 3 followed by purification of the crude product by flash chromatography (hexanes/DCM).

MS (m/z): 407 [M+1]⁺

¹H NMR δ (400 MHz, CDCl₃): 0.69-0.99 (m, 3H), 1.17-1.52 (m, 18H), 1.63-1.82 (m, 2H), 1.95-2.12 (m, 2H), 1.95-2.12 (m, 2H), 2.38 (t, J=8.1 Hz, 2H), 3.42-3.58 (m, 2H), 3.59-3.69 (m, 2H), 3.86 (t, J=6.6 Hz, 2H), 4.29-4.39 (m, 2H), 6.41-6.62 (m, 2H).

### Example 5

### 2,2,2-Trifluoroethyl 4-(dodecyloxy)-1H-pyrrole-2-carboxylate

Obtained (44%) from 4-(dodecyloxy)-1H-pyrrole-2-carboxylic acid (Example 1) and 2,2,2-trifluoroethan-1-ol following the experimental procedure as described in Example 3 followed by purification of the crude product by flash chromatography (hexanes/DCM).

MS (m/z): 378 [M+1]⁺

¹H NMR δ (400 MHz, CDCl₃): 0.80-0.97 (m, 3H), 1.12-1.47 (m, 18H), 1.63-1.83 (m, 2H), 3.88 (t, J=6 Hz, 2H), 4.61 (q, J=8 Hz, 2H), 6.55-6.71 (m, 2H).

### Example 6

### 2-Hydroxyethyl 4-(dodecyloxy)-1H-pyrrole-2-carboxylate

Obtained (64%) from 4-(dodecyloxy)-1H-pyrrole-2-carboxylic acid (Example 1) and ethane-1,2-diol (10 equivalents) following the experimental procedure as described in Example 3 followed by purification of the crude product by flash chromatography (DCM/methanol).

MS (m/z): 340 [M+1]⁺

¹H NMR δ (400 MHz, CDCl₃): 0.84-0.92 (m, 3H), 1.18-1.47 (m, 18H), 1.68-1.79 (m, 2H), 2.05 (t, J=5.9 Hz, 1H), 3.87 (t, J=6 Hz, 2H), 3.90-3.97 (m, 2H), 4.34-4.46 (m, 2H), 6.58 (d, J=2 Hz, 2H), 8.68 (s, 1H).

### Example 7

### 2-(2-(2-(2-Hydroxyethoxy)ethoxy)ethoxy)ethyl 4-(dodecyloxy)-1H-pyrrole-2-carboxylate

Obtained (43%) from 4-(dodecyloxy)-1H-pyrrole-2-carboxylic acid (Example 1) and 2,2′-((oxybis(ethane-2,1-diyl))bis(oxy))bis(ethan-1-ol) (10 equivalents) following the experimental procedure as described in Example 3 followed by purification of the crude product by flash chromatography (hexanes/DCM).

MS (m/z): 472 [M+1]⁺

¹H NMR δ (400 MHz, CDCl₃): 0.76-0.98 (m, 3H), 1.18-1.52 (m, 18H), 1.67-1.85 (m, 2H), 3.24 (s, 1H, OH), 3.62-3.68 (m, 4H), 3.68-3.73 (m, 6H), 3.74-3.80 (m, 4H), 3.86 (t, J=6 Hz, 2H), 4.32-4.55 (m, 2H), 6.46-6.55 (m, 1H), 6.55-6.68 (m, 1H), 9.88 (s, 1H, NH).

### Example 8

### 1-((Isopropoxycarbonyl)oxy)ethyl 4-(dodecyloxy)-1H-pyrrole-2-carboxylate

A mixture of 4-(dodecyloxy)-1H-pyrrole-2-carboxylic acid (Example 1, 80 mg, 0.27 mmol), 1-chloroethyl isopropyl carbonate (45 mg, 0.27 mmol) and triethylamine (94 μL, 0.67 mmol) in AON (2 mL) was heated at 100° C. for 24 h. After cooling to room temperature, the reaction mixture was partitioned between water and DCM. The organic phase was separated and the aqueous phase was washed with DCM (×3). The combined organic extracts were dried over magnesium sulfate, filtered and the solvents were evaporated to dryness. The residue was purified by flash chromatography (DCM/methanol) to yield the title compound (25 mg, 21%).

MS (m/z): 426 [M+1]⁺

¹H NMR δ (400 MHz, CDCl₃): 0.81-0.97 (m, 3H), 1.13-1.50 (m, 24H), 1.60 (d, J=5 Hz, 3H), 1.67-1.85 (m, 2H), 3.86 (t, J=6 Hz, 2H), 4.78-4.99 (m, 1H), 6.48-6.71 (m, 2H), 6.91-7.06 (m, 1H), 8.64 (s, 1H).

### Example 9

### 2-((2-Ethoxy-2-oxoethyl)(methyl)amino)-2-oxoethyl 4-(dodecyloxy)-1H-pyrrole-2-carboxylate

A solution of 4-(dodecyloxy)-1H-pyrrole-2-carboxylic acid (Example 1, 80 mg, 0.27 mmol), ethyl N-(2-chloroacetyl)-N-methylglycinate (Intermediate 2, 63 mg, 0.32 mmol) and triethylamine (38 μL, 0.27 mmol) in ACN (2 mL) was heated at 100° C. for 72 h. After cooling to room temperature, the reaction mixture was partitioned between water and DCM. The organic layer was separated and the aqueous layer was washed with DCM (×3). The combined organic extracts were dried over magnesium sulfate, filtered and the solvents were evaporated to dryness. The residue was purified by flash chromatography (DCM/methanol) and reverse phase chromatography (water/ACN both with 0.5% of formic acid) to yield the title compound (21 mg, 16%).

MS (m/z): 454 [M+1]⁺

¹H NMR δ (400 MHz, CDCl₃): 0.78-0.94 (m, 3H), 1.16-1.49 (m, 21H), 1.66-1.79 (m, 2H), 3.09 (s, 3H), 3.86 (t, J=6 Hz, 2H), 4.14 (s, 2H), 4.19 (t, J=7 Hz, 2H), 4.95 (s, 2H), 6.57 (dd, J=3 and 2 Hz, 1H), 6.61-6.71 (m, 1H), 8.92 (s, 1H).

### Example 10

### 2-((L-valyl)oxy)ethyl 4-(dodecyloxy)-1H-pyrrole-2-carboxylate

A solution of 2-hydroxyethyl 4-(dodecyloxy)-1H-pyrrole-2-carboxylate (Example 6, 109 mg, 0.32 mmol), N-(fert-butoxycarbonyl)-L-valine (84 mg, 0.38 mmol), EDC.HCl (74 mg, 0.38 mmol) and 4-DMAP (98 mg, 0.80 mmol) in DCM (2 mL) was stirred at room temperature for 20 h. The reaction mixture was then partitioned between water and DCM. The organic layer was separated and the aqueous layer was washed with DCM (×3). The combined organic layers were dried over magnesium sulfate, filtered and the solvent was evaporated to dryness. The residue was purified by flash chromatography (DCM/methanol) to yield 2-(((tert-butoxycarbonyl)-L-valyl)oxy)ethyl 4-(dodecyl oxy)-1H-pyrrole-2-carboxylate (111 mg, 63%).

MS (m/z): 539 [M+1]⁺

¹H NMR δ (400 MHz, CDCl₃): 0.80-1.09 (m, 9H), 1.09-1.65 (m, 27H), 1.65-1.90 (m, 2H), 2.03-2.22 (m, 1H), 3.86 (t, J=6 Hz, 2H), 4.12-4.30 (m, 1H), 4.45 (s, 4H), 4.99 (d, J=7 Hz, 1H), 6.48-6.65 (m, 2H), 8.92 (s, 1H).

A mixture of 2-(((tert-butoxycarbonyl)-L-valyl)oxy)ethyl 4-(dodecyloxy)-1H-pyrrole-2-carboxylate (111 mg, 0.20 mmol) and 4M hydrogen chloride solution in dioxane (6.2 mL, 24.8 mmol) was stirred at room temperature for 1 h. The solvent was evaporated and the residue was partitioned between saturated sodium hydrogencarbonate solution and DCM. The organic layer was separated and the aqueous layer was washed with DCM (×2). The combined organic extracts were dried over magnesium sulfate, filtered and the solvent was evaporated to dryness. The residue was purified by flash chromatography (DCM/methanol) to yield the title compound (37 mg, 41%).

MS (m/z): 439 [M+1]⁺

¹H NMR δ (400 MHz, CDCl₃): 0.89 (t, 3H), 0.91 (d, 3H), 0.97 (d, J=7 Hz, 3H), 1.26 (s, 18H), 1.62-1.78 (m, 2H), 1.89-2.10 (m, 1H), 3.32 (d, J=5 Hz, 1H), 3.85 (t, J=6 Hz, 2H), 4.24-4.55 (m, 4H), 6.36-6.69 (m, 2H), 8.85 (s, 1H).

### Example 11

### (5-methyl-2-oxo-1,3-dioxol-4-yl)methyl 4-(dodecyloxy)-1H-pyrrole-2-carboxylate

A mixture of 4-(dodecyloxy)-1H-pyrrole-2-carboxylic acid (Example 1, 80 mg, 0.27 mmol), 4-(bromomethyl)-5-methyl-1,3-dioxol-2-one (63 mg, 0.32 mmol) and potassium carbonate (94 mg, 0.67 mmol) in DMF (2 mL) was stirred at room temperature for 2 h. The reaction mixture was then partitioned between water and DCM. The organic layer was separated and the aqueous layer was washed with DCM (×4). The combined organic extracts were washed with water, dried over magnesium sulfate, filtered and the solvent was evaporated to dryness. The residue was purified by flash chromatography (first using DCM/methanol as eluents and then hexanes/EtOAc) to yield the title compound (20 mg, 18%).

MS (m/z): 408 [M+1]⁺

¹H NMR δ (400 MHz, CDCl₃): 0.70-0.99 (m, 3H), 1.17-1.54 (m, 18H), 1.66-1.92 (m, 2H), 2.21 (s, 3H), 3.86 (t, J=6 Hz, 2H), 5.00 (s, 2H), 6.37-6.71 (m, 2H), 8.70 (s, 1H).

### Example 12

### 4-Decyl-3-fluoro-1H-pyrrole-2-carboxylic acid

To a solution of ethyl 4-decyl-3-fluoro-1H-pyrrole-2-carboxylate (Intermediate 3c, 40 mg, 0.13 mmol) in ethanol (1.5 mL) was added sodium hydroxide (18.8 mg, 0.47 mmol) and the mixture was heated at 80° C. overnight. After cooling to room temperature the solvent was removed in vacuo. Water was added and pH was adjusted to 2 by addition of 1N hydrochloric acid solution. The reaction mixture was then extracted with EtOAc (×3). The combined organic extracts were washed with water and brine, dried over magnesium sulfate, filtered and the solvent was evaporated to yield the title compound as a white solid (28 mg, 77%).

MS (m/z): 270 [M+1]⁺

¹H-NMR δ (400 MHz, DMSO-d6): 0.85 (t, J=6 Hz, 3H), 1.23 (s, 14H), 1.55-1.39 (m, 2H), 2.32 (t, J=7 Hz, 2H), 6.75-6.51 (m, 1H), 11.37-11.11 (m, 1H).

### Example 13

### 3-Fluoro-4-undecyl-1H-pyrrole-2-carboxylic acid

Obtained as a solid (79%) from ethyl 3-fluoro-4-undecyl-1H-pyrrole-2-carboxylate (Intermediate 4c) following the experimental procedure described in Example 1 using methanol as solvent.

MS (m/z) 284 [M+1]⁺

¹H NMR δ (400 MHz, DMSO-d₆) 0.76-0.90 (m, 3H), 1.24 (d, J=9 Hz, 16H), 1.41-1.53 (m, 2H), 2.32 (t, J=7 Hz, 2H), 6.54-6.73 (m, 1H), 11.26 (s, 1H).

### Example 14

### 4-Dodecyl-3-fluoro-1H-pyrrole-2-carboxylic acid

Obtained as a white solid (50%) from ethyl 4-dodecyl-3-fluoro-1H-pyrrole-2-carboxylate (Intermediate 5b) following the experimental procedure described in Example 12.

MS (m/z) 298 [M+1]⁺

¹H NMR δ (400 MHz, DMSO-d₆) 0.76-0.90 (m, 3H), 1.24 (d, J=8 Hz, 17H), 1.39-1.57 (m, 2H), 2.32 (t, J=7 Hz, 2H), 6.56-6.72 (m, 1H), 11.26 (s, 1H).

### Example 15

### 2,2,2-Trifluoroethyl 4-dodecyl-3-fluoro-1H-pyrrole-2-carboxylate

Obtained as a white solid (58%) from 4-dodecyl-3-fluoro-1H-pyrrole-2-carboxylic acid (Example 14) and 2,2,2-trifluoroethanol following the experimental procedure described in Example 3 followed by purification of the crude product by flash chromatography (hexanes/diethyl ether).

¹H-NMR δ (400 MHz, CDCl₃): 0.69-0.98 (m, 3H), 1.15-1.37 (m, 18H), 1.47-1.60 (m, 2H), 2.33-2.51 (m, 2H), 4.65 (q, J=8 Hz, 2H), 6.55-6.70 (m, 1H), 8.41 (s, 1H).

### Example 16

### 2-(2-Ethoxyethoxy)ethyl 4-dodecyl-3-fluoro-1H-pyrrole-2-carboxylate

Obtained as a white solid (18%) from 4-dodecyl-3-fluoro-1H-pyrrole-2-carboxylic acid (Example 14) and 2-(2-ethoxyethoxy)ethanol following the experimental procedure described in Example 3 followed by purification of the crude product by flash chromatography (hexanes/diethyl ether).

MS (m/z): 414 [M+1]⁺

¹H-NMR δ (400 MHz, DMSO-d6): 0.73-0.88 (m, 3H), 1.06 (t, J=7 Hz, 3H), 1.14-1.30 (m, 18H), 1.47 (t, J=7 Hz, 2H), 2.33 (t, J=7 Hz, 2H), 3.46 (s, 4H), 3.52-3.58 (m, 2H), 3.63-3.69 (m, 2H), 4.25-4.31 (m, 2H), 6.68-6.77 (m, 1H), 11.43 (s, 1H).

### Example 17

### 1-((Isopropoxycarbonyl)oxy)ethyl 4-dodecyl-3-fluoro-1H-pyrrole-2-carboxylate

Obtained as a grey solid (46%) from 4-dodecyl-3-fluoro-1H-pyrrole-2-carboxylic acid (Example 14) and 1-chloroethyl isopropyl carbonate following the experimental procedure described in Example 8 followed by purification of the crude product by flash chromatography (hexanes/DCM).

MS (m/z): 428 [M+1]⁺

¹H NMR δ (400 MHz, CDCl₃): 0.82-0.92 (m, 3H), 1.24-1.38 (m, 22H), 1.48-1.57 (m, 2H), 1.61 (d, 3H), 2.34-2.44 (m, 2H), 4.84-4.95 (m, 1H), 6.55-6.64 (m, 1H), 6.97 (q, J=5.5 Hz, 1H), 8.38 (s, 1H).

### Example 18

### 1-(((2-Methoxyethoxy)carbonyl)oxy)ethyl 4-dodecyl-3-fluoro-1H-pyrrole-2-carboxylate

Obtained as a yellow oil (84%) from 4-dodecyl-3-fluoro-1H-pyrrole-2-carboxylic acid (Example 14) and 1-chloroethyl 2-methoxyethyl carbonate (Intermediate 6) following the experimental procedure described in Example 8 followed by purification of the crude product by flash chromatography (hexanes/diethyl ether) and reverse phase chromatography (water/ACN both with 0.5% of formic acid).

MS (m/z): 461 [M+18]⁺

¹H NMR δ (400 MHz, CDCl₃): 0.83-0.91 (m, 3H), 1.19-1.38 (m, 18H), 1.48-1.57 (m, 2H), 1.62 (d, J=5.4 Hz, 3H), 2.34-2.44 (m, 2H), 3.38 (s, 3H), 3.62 (t, J=4.7 Hz, 2H), 4.21-4.38 (m, 2H), 6.56-6.62 (m, 1H), 6.98 (q, J=5.4 Hz, 1H), 8.45 (s, 1H).

### Example 19

### 4-Oxo-3,5,8,11-tetraoxatridecan-2-yl 4-dodecyl-3-fluoro-1H-pyrrole-2-carboxylate

Obtained as a yellow oil (16%) from 4-dodecyl-3-fluoro-1H-pyrrole-2-carboxylic acid (Example 14) and 1-chloroethyl (2-(2-ethoxyethoxy)ethyl) carbonate (Intermediate 7) following the experimental procedure described in Example 8 followed by purification of the crude product by flash chromatography (hexanes/diethyl ether) and reverse phase chromatography (water/ACN both with 0.5% of formic acid).

MS (m/z): 519 [M+1]⁺

¹H-NMR δ (400 MHz, CDCl₃): 0.85-0.91 (m, 3H), 1.21 (t, J=7 Hz, 3H), 1.27 (m, 18H), 1.53 (d, J=7 Hz, 2H), 1.62 (d, J=5 Hz, 3H), 2.40 (t, J=8 Hz, 2H), 3.52 (q, J=7 Hz, 2H), 3.56-3.60 (m, 2H), 3.62-3.67 (m, 2H), 3.70-3.76 (m, 2H), 4.32 (ddd, J=6, 4 and 1 Hz, 2H), 6.55-6.61 (m, 1H), 6.97 (q, J=5 Hz, 1H), 8.48 (s, 1H).

### Example 20

### Ethyl 3-fluoro-4-tridecyl-1H-pyrrole-2-carboxylate

Obtained as a white solid (70%) from ethyl 3-fluoro-4-tridecanoyl-1H-pyrrole-2-carboxylate (Intermediate 8b) following the experimental procedure described in Intermediate 3c followed by purification of the crude product by flash chromatography (hexanes/diethyl ether).

MS (m/z): 340 [M+1]⁺

¹H-NMR δ (400 MHz, CDCl₃): 0.93-0.82 (m, 3H), 1.26 (s, 20H), 1.36 (t, J=7 Hz, 3H), 1.55 (dd, J=13 and 6 Hz, 2H), 2.46-2.34 (m, 2H), 4.33 (q, J=7 Hz, 2H), 6.66-6.41 (m, 1H), 8.41 (brs, 1H).

### Example 21

### 3-Fluoro-4-tridecyl-1H-pyrrole-2-carboxylic acid

To a solution of ethyl 3-fluoro-4-tridecyl-1H-pyrrole-2-carboxylate (Example 20, 2379 mg, 7.01 mmol) in ethanol (60 mL) was added sodium hydroxide (981 mg, 24.53 mmol) and the mixture was heated to reflux overnight. The volatiles were removed under reduced pressure, water was added and pH lowered to 2 by addition of 1N hydrochloric acid solution. The solid formed was filtered, washed with water (×3) and dried to yield the title compound (2097 mg, 95%) as a white solid.

MS (m/z): 312 [M+1]⁺

¹H-NMR δ (400 MHz, DMSO-d₆): 0.92-0.75 (m, 3H), 1.23 (m, 20H), 1.52-1.41 (m, 2H), 2.32 (t, J=7 Hz, 2H), 6.75-6.52 (m, 1H), 11.34-11.16 (m, 1H).

### Example 22

### Methyl 3-fluoro-4-tridecyl-1H-pyrrole-2-carboxylate

To a solution of 3-fluoro-4-tridecyl-1H-pyrrole-2-carboxylic acid (Example 21, 100 mg, 0.32 mmol) in a 2.5:1 mixture of methanol/DCM (2.1 mL) was added DCC (74.2 mg, 0.36 mmol) and 4-DMAP (1.96 mg, 0.02 mmol) and the mixture was stirred at room temperature overnight. The reaction mixture was filtered and the solid was washed with DCM (×3). The combined organic layers were concentrated under reduced pressure and the residue was purified by flash chromatography (hexanes/EtOAc) to yield the title compound (71 mg, 68%) as a white solid.

MS (m/z): 326 [M+1]⁺

¹H-NMR δ (400 MHz, CDCl₃): 0.87 (d, J=7 Hz, 3H), 1.25 (s, 20H), 1.53 (d, J=7 Hz, 2H), 2.41 (t, J=7 Hz, 2H), 3.87 (s, 3H), 6.62-6.49 (m, 1H), 8.39 (brs, 1H).

### Example 23

### Isopropyl 3-fluoro-4-tridecyl-1H-pyrrole-2-carboxylate

To a solution of 3-fluoro-4-tridecyl-1H-pyrrole-2-carboxylic acid (Example 21, 125 mg, 0.4 mmol) in DCM (2.5 mL) were added EDC.HCl (92 mg, 0.48 mmol), 4-DMAP (59 mg, 0.48 mmol) and isopropanol (0.05 mL, 0.6 mmol) and the mixture was stirred at room temperature overnight. The reaction mixture was partitioned between DCM and water. The organic layer was separated and washed with brine, dried over magnesium sulfate, filtered and the solvent evaporated to dryness. The residue was purified by flash chromatography (hexanes/EtOAc) to yield the title compound (22 mg, 16%) as a white solid.

MS (m/z): 354 [M+1]⁺

¹H-NMR δ (400 MHz, CDCl₃): 0.93-0.82 (m, 3H), 1.41-1.19 (m, 23H), 1.60-1.48 (m, 2H), 2.46-2.34 (m, 2H), 5.20 (p, J=6 Hz, 1H), 6.59-6.45 (m, 1H), 8.32 (brs, 1H).

### Example 24

### Tert-butyl 3-fluoro-4-tridecyl-1H-pyrrole-2-carboxylate

To a solution of 3-fluoro-4-tridecyl-1H-pyrrole-2-carboxylic acid (Example 21, 100 mg, 0.32 mmol) in DCM (3 mL) at 0° C. were added oxalyl chloride (0.11 mL, 1.28 mmol) and DMF (4 drops) and the mixture was stirred at room temperature for 3 h. The solvent was evaporated to dryness and the resulting 3-fluoro-4-tridecyl-1H-pyrrole-2-carbonyl chloride (106 mg, 0.32 mmol) and tert-butanol (1.83 mL, 19.28 mmol) were stirred at room temperature for 22 h. The reaction mixture was partitioned between water and DCM. The organic layer was separated and the aqueous layer was washed with DCM (×3). The combined organic phases were dried over magnesium sulfate, filtered and the solvent evaporated to dryness. The residue was purified by flash chromatography (hexanes/EtOAc) to yield the title compound (22 mg, 19%).

MS (m/z): 368 [M+1]⁺

¹H NMR δ (400 MHz, MeOD) 0.86-0.96 (m, 3H), 1.31 (m, 20H), 1.55 (s, 11H), 2.39 (t, J=7 Hz, 2H), 6.55 (d, J=5 Hz, 1H).

### Example 25

### Cyclohexyl 3-fluoro-4-tridecyl-1H-pyrrole-2-carboxylate

To a solution of 3-fluoro-4-tridecyl-1H-pyrrole-2-carboxylic acid (Example 21, 110 mg, 0.35 mmol) in DCM (2 mL) were added EDC.HCl (81 mg, 0.42 mmol) and 4-DMAP (52 mg, 0.42 mmol) followed by cyclohexanol (37 mg, 0.37 mmol) and the mixture was stirred at room temperature for 16 h. The reaction mixture was partitioned between DCM and water. The organic layer was separated and the aqueous layer was washed with DCM. The combined organic extracts were washed with brine, dried over magnesium sulfate, filtered and the solvent evaporated to dryness. The residue was purified by flash chromatography (hexanes/diethyl ether) to yield the title compound (14 mg, 10%) as a white solid.

MS (m/z): 394 [M+1]⁺

¹H-NMR δ (400 MHz, DMSO-d₆): 0.92-0.74 (m, 3H), 1.24 (d, J=9 Hz, 24H), 1.42-1.30 (m, 2H), 1.53-1.42 (m, 4H), 1.85-1.63 (m, 4H), 2.32 (t, J=7 Hz, 2H), 4.95-4.78 (m, 1H), 6.77-6.60 (m, 1H), 11.36 (brs, 1H).

### Example 26

### Benzyl 3-fluoro-4-tridecyl-1H-pyrrole-2-carboxylate

Obtained (48%) from 3-fluoro-4-tridecyl-1H-pyrrole-2-carboxylic acid (Example 21) and phenylmethanol following the experimental procedure described in Example 25 followed by purification of the crude product by flash chromatography (hexanes/DCM).

MS (m/z): 402 [M+1]⁺

¹H NMR δ (400 MHz, CDCl₃): 0.81-0.93 (m, 3H), 1.25 (s, 20H), 1.43-1.56 (m, 2H), 2.34-2.48 (m, 2H), 5.33 (s, 2H), 6.47-6.62 (m, 1H), 7.27-7.52 (m, 5H), 8.36 (s, 1H).

### Example 27

### 2,2,2-Trifluoroethyl 3-fluoro-4-tridecyl-1H-pyrrole-2-carboxylate

Obtained as a white solid (51%) from 3-fluoro-4-tridecyl-1H-pyrrole-2-carboxylic acid (Example 21) and 2,2,2-trifluoroethanol following the experimental procedure described in Example 25 followed by purification of the crude product by flash chromatography (hexanes/EtOAc).

¹H-NMR δ (400 MHz, CDCl₃): 0.87 (t, J=7 Hz, 3H), 1.26 (s, 20H), 1.55 (m, 2H), 2.48-2.38 (m, 2H), 4.65 (q, J=8 Hz, 2H), 6.69-6.59 (m, 1H), 8.41 (brs, 1H).

### Example 28

### 2-(2,5-Dioxopyrrolidin-1-yl)ethyl 3-fluoro-4-tridecyl-1H-pyrrole-2-carboxylate

Obtained as a white solid (40%) from 3-fluoro-4-tridecyl-1H-pyrrole-2-carboxylic acid (Example 21) and 1-(2-hydroxyethyl)pyrrolidine-2,5-dione following the experimental procedure described in Example 25 followed by purification of the crude product by flash chromatography (hexanes/EtOAc).

MS (m/z): 437 [M+1]⁺

¹H-NMR δ (400 MHz, CDCl₃): 0.94-0.79 (m, 3H), 1.26 (s, 20H), 1.55-1.48 (m, 2H), 2.45-2.31 (m, 2H), 2.74 (s, 4H), 3.95-3.83 (m, 2H), 4.45-4.33 (m, 2H), 6.63-6.48 (m, 1H), 8.46 (brs, 1H).

### Example 29

### 2-(2-Oxopyrrolidin-1-yl)ethyl 3-fluoro-4-tridecyl-1H-pyrrole-2-carboxylate

Obtained as a white solid (44%) from 3-fluoro-4-tridecyl-1H-pyrrole-2-carboxylic acid (Example 21) and 1-(2-hydroxyethyl)pyrrolidin-2-one following the experimental procedure described in Example 25 followed by purification of the crude product by flash chromatography (hexanes/EtOAc).

MS (m/z): 423 [M+1]⁺

¹H-NMR δ (400 MHz, CDCl₃): 0.93-0.81 (m, 3H), 1.26 (s, 20H), 1.58-1.49 (m, 2H), 2.04 (p, J=8 Hz, 2H), 2.39 (q, J=8 and 8 Hz, 4H), 3.58-3.49 (m, 2H), 3.71-3.59 (m, 2H), 4.45-4.30 (m, 2H), 6.64-6.50 (m, 1H), 8.61 (brs, 1H).

### Example 30

### (5-Methyl-2-oxo-1,3-dioxol-4-yl)methyl 3-fluoro-4-tridecyl-1H-pyrrole-2-carboxylate

To a solution of 3-fluoro-4-tridecyl-1H-pyrrole-2-carboxylic acid (Example 21, 150 mg, 0.48 mmol) in DMF (3 mL) was added potassium carbonate (166 mg, 1.2 mmol) followed by 4-(bromomethyl)-5-methyl-1,3-dioxol-2-one (112 mg, 0.58 mmol) and the mixture was stirred at 50° C. for 3 h. After cooling to room temperature, the reaction mixture was partitioned between water and toluene. The organic layer was separated and the aqueous phase was washed with toluene. The combined organic extracts were washed with brine, dried over magnesium sulfate, filtered and the solvent was evaporated to dryness. The residue was purified by flash chromatography (hexanes/EtOAc) to yield the title compound (72 mg, 35%) as a white solid.

MS (m/z): 424 [M+1]⁺

¹H-NMR δ (400 MHz, CDCl₃): 0.98-0.75 (m, 3H), 1.25 (s, 20H), 1.53 (d, J=8 Hz, 2H), 2.22 (s, 3H), 2.50-2.36 (m, 2H), 5.03 (s, 2H), 6.64-6.56 (m, 1H), 8.37 (brs, 1H).

### Example 31

### 2-((2-Ethoxy-2-oxoethyl)(methyl)amino)-2-oxoethyl 3-fluoro-4-tridecyl-1H-pyrrole-2-carboxylate

Obtained as a white solid (41%) from 3-fluoro-4-tridecyl-1H-pyrrole-2-carboxylic acid (Example 21) and ethyl N-(2-chloroacetyl)-N-methylglycinate (Intermediate 2) following the experimental procedure described in Example 9 followed by purification of the crude product by flash chromatography (hexanes/EtOAc).

MS (m/z): 469 [M+1]⁺

¹H-NMR δ (400 MHz, CDCl₃): 0.97-0.78 (m, 3H), 1.38-1.20 (m, 23H), 1.61-1.49 (m, 2H), 2.40 (t, J=8 Hz, 2H), 3.06 (s, 3H), 4.11 (s, 2H), 4.20 (q, J=7 Hz, 2H), 4.91 (s, 2H), 6.57 (t, J=4 Hz, 1H), 8.66 (brs, 1H).

### Example 32

### 2-Hydroxyethyl 3-fluoro-4-tridecyl-1H-pyrrole-2-carboxylate

Obtained as a white solid (37%) from 3-fluoro-4-tridecyl-1H-pyrrole-2-carboxylic acid (Example 21) and ethane-1,2-diol (10 equivalents) following the experimental procedure described in Example 25 followed by purification of the crude product by flash chromatography (hexanes/EtOAc).

MS (m/z): 356 [M+1]⁺

¹H-NMR δ (400 MHz, DMSO-d₆): 0.84 (m, 3H), 1.23 (s, 20H), 1.53-1.42 (m, 2H), 2.33 (t, J=7 Hz, 2H), 3.72-3.57 (m, 2H), 4.22-4.12 (m, 2H), 4.79 (m, 1H), 6.79-6.65 (m, 1H), 11.40 (brs, 1H).

### Example 33

### 3-Hydroxypropyl 3-fluoro-4-tridecyl-1H-pyrrole-2-carboxylate

Obtained (54%) from 3-fluoro-4-tridecyl-1H-pyrrole-2-carboxylic acid (Example 21) and propane-1,3-diol (10 equivalents) following the experimental procedure described in Example 25 followed by purification of the crude product by flash chromatography (DCM/methanol then hexanes/EtOAc).

MS (m/z): 370 [M+1]⁺

¹H NMR δ (400 MHz, CDCl₃): 0.84-0.93 (m, 3H), 1.26 (s, 20H), 1.49-1.54 (m, 2H), 1.97 (p, J=6 Hz, 2H), 2.07 (t, J=6 Hz, 1H), 2.36-2.44 (m, 2H), 3.77 (q, J=6 Hz, 2H), 4.38-4.50 (m, 2H), 6.57 (dd, J=4.6 and 3.6 Hz, 1H), 8.38 (s, 1H).

### Example 34

### 4-Hydroxybutyl 3-fluoro-4-tridecyl-1H-pyrrole-2-carboxylate

Obtained as a white solid (27%) from 3-fluoro-4-tridecyl-1H-pyrrole-2-carboxylic acid (Example 21) and butane-1,4-diol (10 equivalents) following the experimental procedure described in Example 25 followed by purification of the crude product by flash chromatography (hexanes/diethyl ether).

MS (m/z): 384 [M+1]⁺.

¹H-NMR δ (400 MHz, CDCl₃): 0.88 (t, J=7 Hz, 3H), 1.22-1.35 (m, 20H), 1.49-1.58 (m, 2H), 1.68-1.75 (m, 2H), 1.80-1.87 (m, 2H), 2.40 (t, J=8 Hz, 2H), 3.72 (t, J=6 Hz, 2H), 4.31 (t, J=6 Hz, 2H), 6.55 (t, J=4 Hz, 1H), 8.44 (brs, 1H).

### Example 35

### 5-Hydroxypentyl 3-fluoro-4-tridecyl-1H-pyrrole-2-carboxylate

Obtained as a white solid (58%) from 3-fluoro-4-tridecyl-1H-pyrrole-2-carboxylic acid (Example 21) and pentane-1,5-diol (10 equivalents) following the experimental procedure described in Example 25 followed by purification of the crude product by flash chromatography (hexanes/diethyl ether).

MS (m/z): 398 [M+1]⁺.

¹H-NMR δ (400 MHz, CDCl₃): 0.88 (t, J=7 Hz, 3H), 1.22-1.35 (m, 20H), 1.46-1.56 (m, 2H), 1.60-1.68 (m, 2H), 1.77 (p, J=7 Hz, 2H), 2.40 (t, J=8 Hz, 2H), 3.68 (t, J=6 Hz, 2H), 4.28 (t, J=7 Hz, 2H), 6.54 (t, J=4 Hz, 1H), 8.41 (br s, 1H).

### Example 36

### 6-Hydroxyhexyl 3-fluoro-4-tridecyl-1H-pyrrole-2-carboxylate

Obtained as a white solid (59%) from 3-fluoro-4-tridecyl-1H-pyrrole-2-carboxylic acid (Example 21) and hexane-1,6-diol (10 equivalents) following the experimental procedure described in Example 25 followed by purification of the crude product by flash chromatography (hexanes/diethyl ether).

MS (m/z): 412 [M+1]⁺.

¹H-NMR δ (400 MHz, CDCl₃): 0.88 (t, J=7 Hz, 3H), 1.22-1.35 (m, 20H), 1.64-1.40 (m, 8H), 1.74 (p, J=7 Hz, 2H), 2.41 (t, J=8 Hz, 2H), 3.66 (t, J=7 Hz, 2H), 4.27 (t, J=7 Hz, 2H), 6.54 (t, J=4 Hz, 1H), 8.42 (br s, 1H).

### Example 37

### 7-Hydroxyheptyl 3-fluoro-4-tridecyl-1H-pyrrole-2-carboxylate

Obtained as a white solid (61%) from 3-fluoro-4-tridecyl-1H-pyrrole-2-carboxylic acid (Example 21) and heptane-1,7-diol (10 equivalents) following the experimental procedure described in Example 25 followed by purification of the crude product by flash chromatography (hexanes/diethyl ether).

MS (m/z): 426 [M+1]⁺.

¹H-NMR δ (400 MHz, CDCl₃): 0.88 (t, J=7 Hz, 3H), 1.22-1.34 (m, 20H), 1.35-1.47 (m, 6H), 1.49-1.62 (m, 4H), 1.73 (p, J=7 Hz, 2H), 2.40 (t, J=8 Hz, 2H), 3.64 (t, J=7 Hz, 2H), 4.26 (t, J=7 Hz, 2H), 6.54 (t, J=4 Hz, 1H), 8.39 (br s, 1H).

### Example 38

### 8-Hydroxyoctyl 3-fluoro-4-tridecyl-1H-pyrrole-2-carboxylate

Obtained as a white solid (63%) from 3-fluoro-4-tridecyl-1H-pyrrole-2-carboxylic acid (Example 21) and octane-1,8-diol (10 equivalents) following the experimental procedure described in Example 25 followed by purification of the crude product by flash chromatography (hexanes/diethyl ether).

MS (m/z): 440 [M+1]⁺.

¹H-NMR δ (400 MHz, CDCl₃): 0.88 (t, J=7 Hz, 3H), 1.21-1.46 (m, 30H), 1.49-1.59 (m, 2H), 1.71 (p, J=7 Hz, 2H), 2.40 (t, J=8 Hz, 2H), 3.64 (t, J=7 Hz, 2H), 4.26 (t, J=7 Hz, 2H), 6.54 (t, J=4 Hz, 1H), 8.44 (br s, 1H).

### Example 39

### 9-Hydroxynonyl 3-fluoro-4-tridecyl-1H-pyrrole-2-carboxylate

Obtained as a white solid (26%) from 3-fluoro-4-tridecyl-1H-pyrrole-2-carboxylic acid (Example 21) and 1,9-nonanediol (10 equivalents) following the experimental procedure described in Example 25 followed by purification of the crude product by flash chromatography (hexanes/EtOAc).

MS (m/z): 454 [M+1]⁺

¹H NMR δ (400 MHz, CDCl₃): 0.80-0.95 (m, 3H), 1.29 (d, J=27.7 Hz, 30H), 1.55 (q, J=8.4, 7.6 Hz, 4H), 1.72 (p, J=6.7 Hz, 2H), 2.35-2.45 (m, 2H), 3.64 (t, J=6.6 Hz, 2H), 4.26 (t, J=6.7 Hz, 2H), 6.51-6.58 (m, 1H), 8.40 (s, 1H).

### Example 40

### 2,3-Dihydroxypropyl 3-fluoro-4-tridecyl-1H-pyrrole-2-carboxylate

Obtained as a white solid (26%) from 3-fluoro-4-tridecyl-1H-pyrrole-2-carboxylic acid (Example 21) and propane-1,2,3-triol (10 equivalents) following the experimental procedure described in Example 25 followed by purification of the crude product by flash chromatography (hexanes/EtOAc).

MS (m/z): 386 [M+1]⁺

¹H-NMR δ (400 MHz, DMSO-d₆): 0.90-0.78 (m, 3H), 1.24 (m, 20H), 1.47 (t, J=7 Hz, 2H), 2.33 (t, J=7 Hz, 2H), 3.71 (q, J=6 Hz, 1H), 4.07 (dd, J=11 and 6 Hz, 1H), 4.19 (d, J=4 Hz, 1H), 4.66 (t, J=6 Hz, 1H), 4.88 (d, J=5 Hz, 1H), 6.76-6.71 (m, 1H), 11.37 (m, 1H)

### Example 41

### 1,3-Dihydroxypropan-2-yl 3-fluoro-4-tridecyl-1H-pyrrole-2-carboxylate

To a solution of 3-fluoro-4-tridecyl-1H-pyrrole-2-carboxylic acid (Example 21,200 mg, 0.64 mmol) in DCM (4 mL) was added EDC.HCl (148 mg, 0.77 mmol) and 4-DMAP (94 mg, 0.77 mmol) followed by 2-phenyl-1,3-dioxan-5-ol (122 mg, 0.67 mmol) and the mixture was stirred at room temperature overnight. The reaction mixture was partitioned between DCM and water. The organic phase was separated and the aqueous phase was washed with DCM. The combined organic layers were washed with water and brine, dried over magnesium sulfate, filtered and the solvent was evaporated to dryness. The residue was purified by flash chromatography (hexanes/EtOAc) to yield 2-phenyl-1,3-dioxan-5-yl 3-fluoro-4-tridecyl-1H-pyrrole-2-carboxylate (79 mg, 26%) as a white solid.

MS (m/z): 474 [M+1]⁺

¹H-NMR δ (400 MHz, CDCl₃): 0.97-0.78 (m, 3H), 1.26 (s, 20H), 1.54 (q, J=9 and 7 Hz, 2H), 2.42 (t, J=8 Hz, 2H), 4.24 (dd, J=13 and 2 Hz, 2H), 4.46-4.33 (m, 2H), 4.98-4.85 (m, 1H), 5.61 (s, 1H), 6.63-6.47 (m, 1H), 7.46-7.33 (m, 3H), 7.59-7.50 (m, 2H), 8.62 (brs, 1H).

To a solution of 2-phenyl-1,3-dioxan-5-yl 3-fluoro-4-tridecyl-1H-pyrrole-2-carboxylate (76 mg, 0.16 mmol) in THF (5 mL) was added 10% Pd/C (8 mg, 0.07 mmol) and the mixture was stirred at room temperature under an hydrogen atmosphere for 16 h. The reaction mixture was filtered over a Celite® pad washing with methanol several times. The filtrate and washings were combined and the solvents were evaporated to yield the title compound (58 mg, 94%) as a white solid.

MS (m/z): 386 [M+1]⁺

¹H-NMR δ (400 MHz, DMSO-d₆): 0.84 (t, J=7 Hz, 3H), 1.32-1.15 (m, 20H), 1.54-1.41 (m, 2H), 2.33 (t, J=7 Hz, 2H), 3.46-3.39 (m, 2H), 3.72 (q, J=6 Hz, 1H), 4.26-4.01 (m, 2H), 6.77-6.70 (m, 1H).

### Example 42

### 2-(2-(2-(2-Hydroxyethoxy)ethoxy)ethoxy)ethyl 3-fluoro-4-tridecyl-1H-pyrrole-2-carboxylate

Obtained as a white wax (40%) from 3-fluoro-4-tridecyl-1H-pyrrole-2-carboxylic acid (Example 21) and 2,2′-((oxybis(ethane-2,1-diyl))bis(oxy))bis(ethan-1-ol) (10 equivalents) following the experimental procedure described in Example 25 followed by purification of the crude product by flash chromatography (hexanes/EtOAc).

MS (m/z): 488 [M+1]⁺

¹H-NMR δ (400 MHz, CDCl₃): 0.98-0.76 (m, 3H), 1.25 (s, 20H), 1.59-1.48 (m, 2H), 2.49-2.30 (m, 2H), 3.29 (s, 1H), 3.85-3.56 (m, 14H), 4.41 (dd, J=5 and 4 Hz, 2H), 6.57-6.40 (m, 1H), 9.71 (brs, 1H).

### Example 43

### 2-(2-Ethoxyethoxy)ethyl 3-fluoro-4-tridecyl-1H-pyrrole-2-carboxylate

Obtained as a white solid (37%) from 3-fluoro-4-tridecyl-1H-pyrrole-2-carboxylic acid (Example 21) and 2-(2-ethoxyethoxy)ethanol following the experimental procedure described in Example 25 followed by purification of the crude product by flash chromatography (hexanes/diethyl ether).

MS (m/z): 428 [M+1]⁺

¹H NMR δ (400 MHz, CDCl₃) 0.96-0.75 (m, 3H), 1.35-1.13 (m, 23H), 1.58-1.49 (m, 2H), 2.40 (t, J=7 Hz, 2H), 3.54 (q, J=7 Hz, 2H), 3.65-3.59 (m, 2H), 3.74-3.68 (m, 2H), 3.84-3.77 (m, 2H), 4.46-4.39 (m, 2H), 6.57-6.52 (m, 1H), 8.54 (brs, 1H).

### Example 44

### 1-((Isopropoxycarbonyl)oxy)ethyl 3-fluoro-4-tridecyl-1H-pyrrole-2-carboxylate

Obtained as a white solid (47%) from 3-fluoro-4-tridecyl-1H-pyrrole-2-carboxylic acid (Example 21) and 1-chloroethyl isopropyl carbonate following the experimental procedure described in Example 8 followed by purification of the crude product by flash chromatography (hexanes/EtOAc).

MS (m/z): 442 [M+1]⁺

¹H NMR δ (400 MHz, CDCl₃): 0.96-0.81 (m, 3H), 1.36-1.20 (m, 26H), 1.56-1.48 (m, 2H), 1.61 (d, J=5 Hz, 3H), 2.40 (t, J=8 Hz, 2H), 4.90 (p, J=6 Hz, 1H), 6.64-6.56 (m, 1H), 6.97 (q, J=5 Hz, 1H), 8.39 (brs, 1H).

### Example 45

### 1-((Tert-butoxycarbonyl)oxy)ethyl 3-fluoro-4-tridecyl-1H-pyrrole-2-carboxylate

Obtained as a grey solid (60%) from 3-fluoro-4-tridecyl-1H-pyrrole-2-carboxylic acid (Example 21) and 1-chloroethyl tert-butyl carbonate (Intermediate 9) following the experimental procedure described in Example 8 followed by purification of the crude product by flash chromatography (hexanes/diethyl ether).

MS (m/z): 456 [M+1]⁺

¹H-NMR δ (400 MHz, CDCl₃): 0.88 (t, J=7 Hz, 3H), 1.20-1.36 (m, 20H), 1.49 (s, 9H), 1.50-1.54 (m, 2H), 1.59 (d, J=5 Hz, 3H), 2.40 (t, J=8 Hz, 2H), 6.58 (t, J=4 Hz, 1H), 6.93 (q, J=5 Hz, 1H), 8.39 (s, 1H).

### Example 46

### 1-(((Nonyloxy)carbonyl)oxy)ethyl 3-fluoro-4-tridecyl-1H-pyrrole-2-carboxylate

Obtained as a white solid (45%) from 3-fluoro-4-tridecyl-1H-pyrrole-2-carboxylic acid (Example 21) and 1-chloroethyl nonyl carbonate (Intermediate 10) following the experimental procedure described in Example 8 followed by purification of the crude product by flash chromatography (hexanes/diethyl ether).

¹H-NMR δ (400 MHz, CDCl₃) 0.83-0.91 (m, 6H), 1.16-1.37 (m, 32H), 1.49-1.56 (m, 2H), 1.62 (d, J=5 Hz, 3H), 1.64-1.70 (m, 2H), 2.40 (t, J=8 Hz, 2H), 4.10-4.21 (m, 2H), 6.54-6.64 (m, 1H), 6.98 (q, J=5 Hz, 1H), 8.43 (s, 1H).

### Example 47

### 1-(((Cyclohexyloxy)carbonyl)oxy)ethyl 3-fluoro-4-tridecyl-1H-pyrrole-2-carboxylate

Obtained as a white solid (63%) from 3-fluoro-4-tridecyl-1H-pyrrole-2-carboxylic acid (Example 21) and 1-chloroethyl cyclohexyl carbonate following the experimental procedure described in Example 8 followed by purification of the crude product by flash chromatography (hexanes/diethyl ether).

MS (m/z): 482 [M+1]⁺.

¹H-NMR δ (400 MHz, CDCl₃): 0.87 (t, J=7 Hz, 3H), 1.19-1.39 (m, 20H), 1.42-1.57 (m, 5H), 1.61 (d, J=5 Hz, 3H), 1.69-1.78 (m, 2H), 1.87-1.96 (m, 2H), 2.39 (t, J=7 Hz, 2H), 4.63 (tt, J=9 and 4 Hz, 1H), 6.60 (dd, J=5 and 4 Hz, 1H), 6.98 (q, J=7 Hz, 1H), 8.73 (br s, 1H).

### Example 48

### 1-(((Benzyloxy)carbonyl)oxy)ethyl 3-fluoro-4-tridecyl-1H-pyrrole-2-carboxylate

Obtained as a white solid (15%) from 3-fluoro-4-tridecyl-1H-pyrrole-2-carboxylic acid (Example 21) and benzyl (1-chloroethyl) carbonate (Intermediate 11) following the experimental procedure described in Example 8 followed by purification of the crude product by flash chromatography (hexanes/diethyl ether).

¹H-NMR δ (400 MHz, CDCl₃): 0.88 (t, J=7 Hz, 3H), 1.21-1.34 (m, 20H), 1.53 (p, J=7 Hz, 2H), 1.62 (d, J=5 Hz, 3H), 2.39 (t, J=7 Hz, 2H), 5.15 (d, J=12 Hz, 1H), 5.21 (d, J=12 Hz, 1H), 6.59 (t, J=4 Hz, 1H), 7.00 (q, J=5 Hz, 1H), 7.31-7.40 (m, 5H), 8.48 (s, 1H).

### Example 49

### 1-(((2-Methoxyethoxy)carbonyl)oxy)ethyl 3-fluoro-4-tridecyl-1H-pyrrole-2-carboxylate

Obtained as a clear oil (22%) from 3-fluoro-4-tridecyl-1H-pyrrole-2-carboxylic acid (Example 21) and 1-chloroethyl (2-methoxyethyl) carbonate (Intermediate 6) following the experimental procedure described in Example 8 followed by purification of the crude product by flash chromatography (hexanes/diethyl ether).

¹H-NMR δ (400 MHz, CDCl₃): 0.88 (t, J=7 Hz, 3H), 1.21-1.34 (m, 20H), 1.53 (p, J=7 Hz, 2H), 1.61 (d, J=5 Hz, 3H), 2.39 (t, J=7 Hz, 2H), 3.37 (s, 3H), 4.25-4.34 (m, 1H), 6.59 (t, J=4 Hz, 1H), 6.98 (q, J=5 Hz, 1H), 8.49 (s, 1H).

### Example 50

### 1-(((3-Hydroxypropoxy)carbonyl)oxy)ethyl 3-fluoro-4-tridecyl-1H-pyrrole-2-carboxylate

A mixture of 3-fluoro-4-tridecyl-1H-pyrrole-2-carboxylic acid (Example 21, 200 mg, 0.64 mmol), 3-(benzyloxy)propyl (1-chloroethyl) carbonate (Intermediate 12, 210 mg, 0.77 mmol) and triethylamine (0.22 mL, 1.60 mmol) in AON (5 mL) was heated at 100° C. for 20 h. After cooling to room temperature, the mixture was partitioned between water and DCM. The organic layer was separated and the aqueous layer was washed with DCM (×2). The combined organic phases were dried over magnesium sulfate, filtered and the solvent was evaporated to dryness. The residue was purified by flash chromatography (hexanes/diethyl ether) to yield 1-(((3-(benzyloxy)propoxy)carbonyl)oxy)ethyl 3-fluoro-4-tridecyl-1H-pyrrole-2-carboxylate (46 mg, 13%)

MS (m/z): 565 [M+18]⁺

¹H NMR δ (400 MHz, CDCl₃): 0.88 (t, J=7 Hz, 3H), 1.27 (d, J=12 Hz, 20H), 1.46-1.57 (m, 2H), 1.61 (d, J=5 Hz, 3H), 1.90-2.07 (m, 2H), 2.39 (t, J=7 Hz, 2H), 3.56 (t, J=6 Hz, 2H), 4.30 (t, J=6 Hz, 2H), 4.49 (s, 2H), 6.46-6.74 (m, 1H), 6.97 (q, J=5 Hz, 1H), 7.28-7.37 (m, 5H), 8.36 (s, 1H).

To a solution of (1-(((3-(benzyloxy)propoxy)carbonyl)oxy)ethyl 3-fluoro-4-tridecyl-1H-pyrrole-2-carboxylate (46 mg, 0.08 mmol) in THF (4 mL) was added 10% Pd/C (9 mg) and the mixture was stirred under a hydrogen atmosphere at room temperature for 4 h. The mixture was filtered through a Celite® pad and the filtrate was evaporated to dryness. The residue was purified by reverse phase chromatography (water/ACN both with 0.5% of formic acid) to yield the title compound (29 mg, 75%).

MS (m/z): 475 [M+18]⁺

¹H NMR δ (400 MHz, CDCl₃): 0.88 (t, J=6.8 Hz, 3H), 1.27 (d, J=12.6 Hz, 20H), 1.45-1.58 (m, 2H), 1.62 (d, J=5 Hz, 3H), 1.92 (p, J=6 Hz, 2H), 2.40 (t, J=7 Hz, 2H), 3.74 (t, J=6 Hz, 2H), 4.33 (hept, J=5.9 and 5.4 Hz, 2H), 6.55-6.64 (m, 1H), 6.97 (q, J=5.5 Hz, 1H), 8.45 (s, 1H).

### Example 51

### 4-Oxo-3,5,8,11-tetraoxatridecan-2-yl 3-fluoro-4-tridecyl-1H-pyrrole-2-carboxylate

Obtained as a colourless oil (27%) from 3-fluoro-4-tridecyl-1H-pyrrole-2-carboxylic acid (Example 21) and 1-chloroethyl (2-(2-ethoxyethoxy)ethyl) carbonate (Intermediate 7) following the experimental procedure described in Example 8 followed by purification of the crude product by flash chromatography (hexanes/EtOAc).

MS (m/z): 516 [M+1]⁺

¹H-NMR δ (400 MHz, CDCl₃): 5.91-0.84 (m, 3H), 1.33-1.16 (m, 23H), 1.54-1.48 (m, 2H), 1.62 (d, J=5 Hz, 3H), 2.44-2.37 (m, 2H), 3.52 (q, J=7 Hz, 2H), 3.61-3.56 (m, 2H), 3.67-3.62 (m, 2H), 3.77-3.70 (m, 2H), 4.37-4.27 (m, 2H), 6.61-6.56 (m, 1H), 6.98 (q, J=5 Hz, 1H), 8.43 (brs, 1H).

### Example 52

### 3-Fluoro-4-tetradecyl-1H-pyrrole-2-carboxylic acid

Obtained as a white solid (89%) from ethyl 3-fluoro-4-tetradecyl-1H-pyrrole-2-carboxylate (Intermediate 13c) following the experimental procedure described in Example 12.

MS (m/z): 326 [M+1]⁺

¹H NMR δ (400 MHz, DMSO-d₆): 0.82-0.89 (m, 3H), 1.23 (s, 22H), 1.42-1.52 (m, 2H), 2.32 (t, J=7.5 Hz, 2H), 6.63-6.67 (m, 1H), 11.25 (s, 1H).

### Example 53

### 3-Fluoro-4-pentadecyl-1H-pyrrole-2-carboxylic acid

Obtained as a white solid (51%) from ethyl 3-fluoro-4-pentadecyl-1H-pyrrole-2-carboxylate (Intermediate 14b) following the experimental procedure described in Example 21.

MS (m/z): 338 [M−1]⁺.

¹H-NMR δ (600 MHz, DMSO-d6): 0.83 (t, J=7.0 Hz, 3H), 1.17-1.28 (m, 24H), 1.41-1.50 (m, 2H), 2.30 (t, J=7.5 Hz, 2H), 6.63 (t, J=4.1 Hz, 1H), 11.25 (bs, 1H), 12.31 (s, 1H).

### Example 54

### 3-Fluoro-4-heptadecyl-1H-pyrrole-2-carboxylic acid

Obtained as a white solid (77%) from ethyl 3-fluoro-4-heptadecyl-1H-pyrrole-2-carboxylate (Intermediate 15b) following the experimental procedure described in Example 21.

MS (m/z): 366 [M+1]⁺.

¹H-NMR δ (400 MHz, DMSO-d6): 0.85 (t, J=6.8 Hz, 2H), 1.17-1.32 (m, 28H), 1.40-1.52 (m, 2H), 2.31 (t, J=7.4 Hz, 2H), 6.61 (s, 1H), 11.18 (s, 1H).

### Example 55

### 5-Dodecyl-3-fluoro-1H-pyrrole-2-carboxylic acid

Obtained as a dark oil (29%) from ethyl 5-dodecyl-3-fluoro-1H-pyrrole-2-carboxylate (Intermediate 16b) following the experimental procedure described in Example 12 followed by purification of the crude product by reverse phase chromatography (water/ACN both with 0.5% of formic acid).

MS (m/z): 298 [M+1]⁺

¹H-NMR δ (400 MHz, CDCl₃): 0.88 (t, J=7 Hz, 3H), 1.28 (m, 18H), 1.52-1.70 (m, 2H), 2.55 (t, J=7 Hz, 2H), 5.77 (s, 1H), 8.54 (s, 1H).

### Example 56

### 3-Chloro-4-decyl-1H-pyrrole-2-carboxylic acid

To a solution of methyl 3-chloro-4-decyl-1H-pyrrole-2-carboxylate (Intermediate 17b, 174 mg, 0.58 mmol) in ethanol (3 mL) and water (0.6 mL) was added lithium hydroxide monohydrate (97 mg, 2.32 mmol) and the reaction was stirred at 78° C. for 2 h. The volatiles were partially removed under reduced pressure, water was added and the pH was adjusted to 1-2 by addition of 1N hydrochloric acid solution. The precipitate was filtered, rinsed with water and dried to yield the title compound (130 mg, 78%) as a white solid.

MS (m/z): 286/288 [M+1/M+3]⁺

¹H-NMR δ (400 MHz, DMSO-d6): 0.84 (t, J=6 Hz, 3H), 1.23 (s, 14H), 1.53-1.41 (m, 2H), 2.34 (t, J=7 Hz, 2H), 6.79 (s, 1H), 11.64 (s, 1H).

### Example 57

### 3-Chloro-4-undecyl-1H-pyrrole-2-carboxylic acid

Obtained (80%) from methyl 3-chloro-4-undecyl-1H-pyrrole-2-carboxylate (Intermediate 18b) following the experimental procedure described in Example 56.

MS (m/z): 300, 302 [M+1/M+3]⁺

¹H NMR δ (400 MHz, DMSO-d₆): 0.85 (t, J=7 Hz, 3H), 1.18-1.35 (m, 16H), 1.48 (p, J=8 and 7 Hz, 2H), 2.34 (t, J=8 Hz, 2H), 6.80 (d, J=3 Hz, 1H), 11.68 (s, 1H).

### Example 58

### 3-Chloro-4-dodecyl-1H-pyrrole-2-carboxylic acid

Obtained as a white solid (20%) from methyl 3-chloro-4-dodecyl-1H-pyrrole-2-carboxylate (Intermediate 19b) following the procedure described in Example 56. The resulting brown solid was triturated with diethyl ether, filtered and dried to obtain the title compound.

MS (m/z): 314 [M+1]⁺.

¹H NMR δ (400 MHz, DMSO-d₆): 0.86 (t, J=6.8 Hz, 3H), 1.26 (m, J=8.0 Hz, 18H), 1.53-1.40 (m, 2H), 2.38-2.27 (m, 2H), 6.50 (s, 1H), 8.25 (s, 1H), 10.95 (s, 1H).

### Example 59

### 9-Hydroxynonyl 3-chloro-4-dodecyl-1H-pyrrole-2-carboxylate

Obtained as a white solid (42%) from 3-chloro-4-dodecyl-1H-pyrrole-2-carboxylic acid (Example 58) and nonane-1,9-diol (10 equivalents) following the experimental procedure described in Example 25.

MS (m/z): 457 [M+1]⁺.

¹H-NMR δ (400 MHz, CDCl₃): 0.85-0.91 (m, 3H), 1.22-1.27 (m, 12H), 1.30-1.36 (m, 10H), 1.40-1.49 (m, 2H), 1.53-1.58 (m, 8H), 1.67-1.79 (m, 2H), 2.38-2.48 (m, 2H), 3.64 (t, J=6.6 Hz, 2H), 4.28 (t, J=6.6 Hz, 2H), 6.69 (d, J=3.2 Hz, 1H), 8.87 (bs, 1H).

### Example 60

### 2-(2,5-dioxopyrrolidin-1-yl)ethyl 3-chloro-4-dodecyl-1H-pyrrole-2-carboxylate

Obtained as a colourless oil (36%) from 3-chloro-4-dodecyl-1H-pyrrole-2-carboxylic acid (Example 58) and 1-(2-hydroxyethyl)pyrrolidine-2,5-dione following the procedure described in Example 25.

MS (m/z): 439 [M+1]⁺.

¹H NMR δ (400 MHz, CDCl₃): 0.88 (t, J=6.8 Hz, 3H), 1.28 (d, J=15.8 Hz, 18H), 1.57-1.44 (m, 2H), 1.60 (d, J=9.9 Hz, 4H), 2.51-2.28 (m, 2H), 3.99-3.79 (m, 2H), 4.46-4.34 (m, 2H), 6.70 (s, 1H), 9.06 (brs, 1H).

### Example 61

### 3-Chloro-4-tridecyl-1H-pyrrole-2-carboxylic acid

To a solution of methyl 3-chloro-4-tridecyl-1H-pyrrole-2-carboxylate (Intermediate 20b, 35 mg, 0.10 mmol) in methanol (1.5 mL) was added aqueous 2N sodium hydroxide solution (0.41 mL, 0.82 mmol) and the mixture was stirred overnight at 45° C. Methanol was removed under reduced pressure, water was added and the pH was adjusted to 2 by addition of 2N hydrochloric acid solution. The white solid formed was separated by filtration, washed with water and dried to give the title compound (25 mg, 75%).

MS (m/z): 328 [M+1]⁺.

¹H-NMR δ (600 MHz, DMSO-d₆): 0.83 (t, J=7.1 Hz, 3H), 1.22-1.26 (m, 20H), 1.41-1.44 (m, 2H), 2.21-2.29 (m, 2H), 6.32 (s, 1H), 10.5 (brs, 1H).

### Example 62

### 3-Chloro-4-pentadecyl-1H-pyrrole-2-carboxylic acid

Obtained as a white solid (29%) from methyl 3-chloro-4-pentadecyl-1H-pyrrole-2-carboxylate (Intermediate 21b) following the procedure described in Example 56.

MS (m/z): 356 [M+1]⁺.

¹H NMR δ (400 MHz, DMSO-d₆): 0.91-0.79 (m, 3H), 1.23 (m, 24H), 1.52-1.43 (m, 2H), 2.40-2.29 (m, 2H), 6.77 (s, 1H).

### Example 63

### 3-Chloro-4-hexadecyl-1H-pyrrole-2-carboxylic acid

Obtained as a white solid (66%) from methyl 3-chloro-4-hexadecyl-1H-pyrrole-2-carboxylate (Intermediate 22b) following the procedure described in Example 56.

MS (m/z): 370 [M+1]⁺.

¹H NMR δ (400 MHz, DMSO-d₆): 0.84 (t, J=7 Hz, 3H), 1.29-1.18 (m, 26H), 1.47 (p, J=2 Hz 2H), 2.34 (t, J=8 Hz, 2H), 6.78 (s, 1H), 11.66 (br s, 1H).

### Example 64

### 3-Chloro-5-undecyl-1H-pyrrole-2-carboxylic acid

To a solution of methyl 3-chloro-5-undecyl-1H-pyrrole-2-carboxylate (Intermediate 23b, 30 mg, 0.095 mmol) in ethanol (1.5 mL) and water (0.25 mL) was added lithium hydroxide monohydrate (21 mg, 0.5 mmol) and the mixture was heated at 80° C. for 20 h. Solvent was removed, EtOAc and water were added and the pH was made acidic by addition of 1N hydrochloric acid solution. Phases were separated and the organic phase was dried over magnesium sulfate, filtered and solvent was evaporated. Purification of the residue by reverse flash chromatography (water/ACN both containing 0.01% of formic acid) gave the title compound (14 mg, 39%) as a white solid.

MS (m/z): 300/302 [M+1]⁺.

¹H-NMR δ (400 MHz, CDCl₃): 0.83-0.94 (m, 3H), 1.19-1.40 (m, 16H), 1.54-1.67 (m, 2H), 2.51-2.61 (m, 2H), 6.02 (br s, 1H), 8.92 (br s, 1H).

### Example 65

### 3-Chloro-5-dodecyl-1H-pyrrole-2-carboxylic acid

Obtained as a white solid (33%) from ethyl 3-chloro-5-dodecyl-1H-pyrrole-2-carboxylate (Intermediate 24e) following the procedure described in Example 64 followed by purification of the crude product by reverse phase chromatography (water/ACN both containing 0.01% of formic acid).

MS (m/z): 314/316 [M+1/M+3]⁺.

¹H-NMR δ (400 MHz, CDCl₃): 0.88 (t, J=7 Hz, 3H), 1.22-1.36 (m, 18H), 1.61 (p, J=8 Hz, 2H), 2.57 (t, J=8 Hz, 2H), 6.03 (d, J=3 Hz, 1H), 8.87 (brs, 1H).

### Example 66

### 3-Chloro-5-tridecyl-1H-pyrrole-2-carboxylic acid

Obtained as a white solid (26%) from methyl 3-chloro-5-tridecyl-1H-pyrrole-2-carboxylate (Intermediate 25b) following the procedure described in Example 12 followed by purification of the crude product by flash chromatography (DCM/methanol).

MS (m/z): 328/330 [M+1, Cl]⁺

¹H NMR δ (400 MHz, CDCl₃): 0.86-0.90 (m, 3H), 1.15-1.26 (m, 20H), 1.49-1.67 (m, 2H), 2.57 (t, J=8 Hz, 2H), 6.02 (s, 1H), 8.82 (s, 1H).

### Example 67

### 3-Chloro-5-tetradecyl-1H-pyrrole-2-carboxylic acid

Obtained as a white solid (69%) from methyl 3-chloro-5-tetradecyl-1H-pyrrole-2-carboxylate (Intermediate 26b) following the procedure described in Example 64 followed by purification of the crude product by reverse phase chromatography (water/ACN both containing 0.01% of formic acid).

MS (m/z): 342/344 [M+1]⁺.

¹H-NMR δ (400 MHz, CDCl₃): 0.88 (t, J=7 Hz, 3H), 1.20-1.36 (m, 22H), 1.61 (p, J=8 Hz, 2H), 2.57 (t, J=8 Hz, 2H), 6.02 (d, J=3 Hz, 1H), 8.86 (brs, 1H).

### Example 68

### 3-Bromo-4-tridecyl-1H-pyrrole-2-carboxylic acid

Obtained as a white solid (13%) from methyl 3-bromo-4-tridecyl-1H-pyrrole-2-carboxylate (Intermediate 27b) following the procedure described in Example 12. The crude product obtained was washed with diethyl ether and filtered to give the title compound.

MS (m/z): 370, 372 [M−1]⁺.

¹H NMR δ (400 MHz, DMSO-d₆+CDCl₃): 0.88 (t, J=6.7 Hz, 3H), 1.26 (m, 20H), 1.47 (m, 2H), 2.32 (m, 2H), 6.51 (s, 1H), 10.93 (s, 1H).

### Example 69

### 1-Butyl-3-fluoro-4-tridecyl-1H-pyrrole-2-carboxylic acid

To a suspension of sodium hydride (60% dispersion in paraffin oil, 30 mg, 0.75 mmol) in DMF (2 mL) at 0° C. was added ethyl 3-fluoro-4-tridecyl-1H-pyrrole-2-carboxylate (Example 20, 200 mg, 0.58 mmol) and the resulting solution was stirred at 0° C. for 20 min. 1-Iodobutane (135 mg, 0.73 mmol) was added and the solution was stirred at room temperature for 1 h. The reaction mixture was poured into water and extracted with DCM (×3). The combined organic extracts were washed with water (×3) and brine, dried over magnesium sulfate and the solvent was removed under reduced pressure. The residue was purified using SP1® Purification System (DCM/methanol) to give ethyl 1-butyl-3-fluoro-4-tridecyl-1H-pyrrole-2-carboxylate (75 mg, 33%) as a colourless oil.

MS (m/z): 396 [M+1]⁺.

1-Butyl-3-fluoro-4-tridecyl-1H-pyrrole-2-carboxylic acid was obtained as a white solid (22%) from ethyl 1-butyl-3-fluoro-4-tridecyl-1H-pyrrole-2-carboxylate following the procedure described in Example 21. The crude product was triturated with hexane to give the title compound.

MS (m/z): 368 [M+1]⁺.

¹H NMR δ (600 MHz, CDCl₃): 0.87 (t, J=7.0 Hz, 3H), 0.91 (t, J=7.4 Hz, 3H), 1.27 (d, J=23.3 Hz, 20H), 1.52 (dt, J=14.8, 7.5 Hz, 4H), 1.72-1.64 (m, 2H), 2.38 (t, J=7.6 Hz, 2H), 4.15 (t, J=7.1 Hz, 2H), 6.51 (d, J=5.5 Hz, 1H).

### Example 70

### 3-Fluoro-1-isopropyl-4-tridecyl-1H-pyrrole-2-carboxylic acid

Obtained as a white solid (4%) from ethyl 3-fluoro-4-tridecyl-1H-pyrrole-2-carboxylate (Example 20) and 2-iodopropane following the procedure described in Example 69.

MS (m/z): 354 [M+1]⁺.

¹H NMR δ (600 MHz, CDCl₃): 0.88 (t, J=7.0 Hz, 3H), 1.25 (s, 20H), 1.38 (d, J=6.7 Hz, 6H), 1.53 (dq, J=17.5, 10.1, 8.8 Hz, 2H), 2.39 (t, 2H), 5.29 (m, J=6.6 Hz, 1H), 6.70 (d, J=5.5 Hz, 1H).

### Example 71

### 4-(Decyloxy)-3-fluoro-1H-pyrrole-2-carboxylic acid

Obtained as an off-white solid (19%) from ethyl 4-(decyloxy)-3-fluoro-1H-pyrrole-2-carboxylate (Intermediate 28d) following the experimental procedure described in Example 21. The solid was triturated with methanol, filtered and dried to yield the title compound.

MS (m/z): 284 [M−1]⁻.

¹H-NMR δ (400 MHz, DMSO-d6): 0.82-0.91 (m, 3H), 1.22-1.31 (m, 12H), 1.32-1.40 (m, 2H), 1.58-1.69 (m, 2H), 3.83 (t, J=6.5 Hz, 2H), 6.57 (s, 1H), 10.98 (bs, 1H).

### Example 72

### 3-Fluoro-4-(undecyloxy)-1H-pyrrole-2-carboxylic acid

Obtained as a white solid (20%) from ethyl 3-fluoro-4-(undecyloxy)-1H-pyrrole-2-carboxylate (Intermediate 29) following the experimental procedure described in Example 21. The crude solid was triturated with methanol, filtered and dried to yield the title compound.

MS (m/z): 298 [M−1]⁻.

¹H-NMR δ (300 MHz, DMSO-d6): 0.85 (t, J=6.0 Hz, 3H), 1.17-1.43 (m, 16H), 1.55-1.71 (m, 2H), 3.84 (t, J=6.4 Hz, 2H), 6.62 (s, 3H), 11.09 (bs, 1H).

### Example 73

### 4-(Dodecyloxy)-3-fluoro-1H-pyrrole-2-carboxylic acid

Obtained as a white solid (81%) from ethyl 4-(dodecyloxy)-3-fluoro-1H-pyrrole-2-carboxylate (Intermediate 30) following the experimental procedure described in Example 21. The crude solid was triturated with hexanes and diethyl ether, filtered and dried to yield the title compound.

MS (m/z): 312 [M−1]⁻.

¹H-NMR δ (400 MHz, CDCl₃): 0.83 (t, J=6.8 Hz, 3H), 1.18-1.29 (m, 16H), 1.31-1.35 (m, 2H), 1.58-1.65 (m, 2H), 3.81 (t, J=6.4 Hz, 2H), 6.59 (s, 1H), 11.06 (s, 1H), 12.51 (s, 1H).

### Example 74

### 3-Fluoro-4-(tridecyloxy)-1H-pyrrole-2-carboxylic acid

Obtained (30%) from ethyl 3-fluoro-4-(tetradecyloxy)-1H-pyrrole-2-carboxylate (Intermediate 31) following the experimental procedure described in Example 21 followed by purification of the crude product by reverse phase chromatography (water/ACN both containing 0.01% of formic acid).

MS (m/z): 328 [M+1]⁺

¹H NMR δ (400 MHz, CDCl₃): 0.85 (t, J=6.9 Hz, 3H), 1.16-1.43 (m, 20H), 1.71 (dt, J=14.6, 6.7 Hz, 2H), 3.89 (t, J=6.6 Hz, 2H), 6.45 (d, J=4.4 Hz, 1H).

### Example 75

### 3-Fluoro-4-(tetradecyloxy)-1H-pyrrole-2-carboxylic acid

Obtained as an off-white solid (72%) from ethyl 3-fluoro-4-tridecoxy-1H-pyrrole-2-carboxylate (Intermediate 32) following the experimental procedure described in Example 21.

MS (m/z): 340 [M−1]⁺.

¹H-NMR δ (400 MHz, CDCl₃): 0.82-0.88 (m, 3H), 1.20-1.30 (m, 20H), 1.31-1.41 (m, 2H), 1.63 (p, J=6.5 Hz, 2H), 3.84 (t, J=6.5 Hz, 2H), 6.61 (t, J=4.0 Hz, 1H), 11.08 (bs, 1H), 12.48 (bs, 1H).

### Example 76

### 4-(Dodecylthio)-3-fluoro-1H-pyrrole-2-carboxylic acid

Obtained as an off-white solid (58%) from ethyl 4-(dodecylthio)-3-fluoro-1H-pyrrole-2-carboxylate (Intermediate 33b) following the experimental procedure described in Example 21.

MS (m/z): 328 [M−1]⁻.

¹H-NMR δ (400 MHz, DMSO-d6): 0.82-0.88 (m, 3H), 1.18-1.26 (m, 18H), 1.28-1.36 (m, 2H), 1.39-1.50 (m, 2H), 2.58 (t, J=7.2 Hz, 2H), 6.93 (d, J=3.6 Hz, 1H), 11.79 (bs, 1H), 12.68 (bs, 1H).

### Example 77

### 3-chloro-4-(nonyloxy)-1H-pyrrole-2-carboxylic acid

Obtained (75%) from methyl 3-chloro-4-(nonyloxy)-1H-pyrrole-2-carboxylate (Intermediate 34d) following the experimental procedure described in Example 12.

¹H NMR δ (400 MHz, CDCl₃): 0.77-1.05 (m, 3H), 1.22-1.54 (m, 12H), 1.73 (p, J=6.5 Hz, 2H), 3.89 (t, J=6.4 Hz, 2H), 6.62 (s, 1H).

### Example 78

### 3-Chloro-4-(decyloxy)-1H-pyrrole-2-carboxylic acid

Obtained as an off-white solid (70%) from methyl 3-chloro-4-(decyloxy)-1H-pyrrole-2-carboxylate (Intermediate 35) following the experimental procedure described in Example 21.

MS (m/z): 300 [M−1]⁻.

¹H-NMR δ (400 MHz, DMSO-d6): 0.83-0.88 (m, 3H), 1.20-1.34 (m, 12H), 1.33-1.40 (m, 2H), 1.65 (p, J=6.6 Hz, 2H), 3.84 (t, J=6.6 Hz, 2H), 6.71 (d, J=3.5 Hz, 1H), 11.52 (bs, 1H), 12.60 (bs, 1H).

### Example 79

### 3-Chloro-4-(undecyloxy)-1H-pyrrole-2-carboxylic acid

Obtained as an off-white solid (43%) from methyl 3-chloro-4-(undecyloxy)-1H-pyrrole-2-carboxylate (Intermediate 36) following the experimental procedure described in Example 21.

MS (m/z): 314 [M−1]⁻.

¹H-NMR δ (400 MHz, DMSO-d6): 0.83-0.88 (m, 3H), 1.21-1.32 (m, 16H), 1.32-1.45 (m, 2H), 1.64 (p, J=6.6 Hz, 2H), 3.84 (t, J=6.6 Hz, 2H), 6.71 (d, J=2.6 Hz, 1H), 11.51 (bs, 1H), 12.61 (bs, 1H).

### Example 80

### 3-Chloro-4-(dodecyloxy)-1H-pyrrole-2-carboxylic acid

Obtained as an off-white solid (67%) from methyl 3-chloro-4-(dodecyloxy)-1H-pyrrole-2-carboxylate (Intermediate 37) following the experimental procedure described in Example 21.

MS (m/z): 328 [M−1]⁻.

¹H-NMR δ (400 MHz, DMSO-d6): 0.83-0.89 (m, 3H), 1.20-1.33 (m, 16H), 1.31-1.41 (m, 2H), 1.63 (p, J=6.6 Hz, 2H), 3.78 (t, J=6.6 Hz, 2H), 6.39 (s, 1H), 10.79 (bs, 1H).

### Example 81

### 2,2,2-Trifluoroethyl 3-chloro-4-(dodecyloxy)-1H-pyrrole-2-carboxylate

Obtained as a yellow solid (34%) from 3-chloro-4-(dodecyloxy)-1H-pyrrole-2-carboxylic acid (Example 80) and 2,2,2-trifluoroethan-1-ol following the experimental procedure described in Example 25 followed by purification of the crude product by flash chromatography (hexanes/DCM).

¹H NMR δ (400 MHz, CDCl₃): 0.84-0.97 (m, 3H), 1.16-1.50 (m, 18H), 1.71-1.86 (m, 2H), 3.91 (t, J=6.6 Hz, 2H), 4.66 (q, J=8.4 Hz, 2H), 6.60 (d, J=3.5 Hz, 1H), 8.61 (s, 1H).

### Example 82

### 9-Hydroxynonyl 3-chloro-4-(dodecyloxy)-1H-pyrrole-2-carboxylate

Obtained as a grey solid (30%) from 3-chloro-4-(dodecyloxy)-1H-pyrrole-2-carboxylic acid (Example 80) and nonane-1,9-diol (10 equivalents) following the experimental procedure described in Example 25 followed by purification of the crude product by flash chromatography (DCM/methanol).

¹H NMR δ (400 MHz, CDCl₃): 0.84-0.93 (m, 3H), 1.17-1.49 (m, 28H), 1.49-1.63 (m, 2H), 1.66-1.84 (m, 4H), 3.59-3.70 (m, 2H), 3.89 (t, J=6.7 Hz, 2H), 4.29 (t, J=6.6 Hz, 2H), 6.51 (d, J=3.4 Hz, 1H), 8.63 (s, 1H).

### Example 83

### 2-(2-Ethoxyethoxy)ethyl 3-chloro-4-(dodecyloxy)-1H-pyrrole-2-carboxylate

Obtained as a grey solid (10%) from 3-chloro-4-(dodecyloxy)-1H-pyrrole-2-carboxylic acid (Example 80) and 2-(2-ethoxyethoxy)ethan-1-ol following the experimental procedure described in Example 25 followed by purification of the crude product by flash chromatography (hexanes/DCM).

MS (m/z): 446 [M+1]⁺

¹H NMR δ (400 MHz, CDCl₃) 0.84-0.94 (m, 3H), 1.15-1.51 (m, 21H), 1.76 (dt, J=14.5, 6.6 Hz, 2H), 3.54 (q, J=7.0 Hz, 2H), 3.58-3.65 (m, 2H), 3.68-3.73 (m, 2H), 3.78-3.85 (m, 2H), 3.89 (t, J=6.7 Hz, 2H), 4.42-4.46 (m, 2H), 6.50 (d, J=3.5 Hz, 1H), 9.04 (s, 1H).

### Example 84

### 2,3-Dihydroxypropyl 3-chloro-4-(dodecyloxy)-1H-pyrrole-2-carboxylate

Obtained (17%) from 3-chloro-4-(dodecyloxy)-1H-pyrrole-2-carboxylic acid (Example 80) and propane-1,2,3-triol (10 equivalents) following the experimental procedure described in Example 25 followed by purification of the crude product by flash chromatography (diethyl ether/methanol).

MS (m/z): 404/406 [M+1/M+3]⁺.

¹H-NMR δ (400 MHz, CDCl₃): 0.88 (t, J=7 Hz, 3H), 1.20-1.37 (m, 16H), 1.38-1.48 (m, 2H), 1.76 (p, J=7 Hz, 2H), 2.12 (brs, 1H), 3.72 (dd, J=11 and 6 Hz, 1H), 3.78 (dd, J=11 and 4 Hz, 1H), 3.89 (t, J=7 Hz, 2H), 4.05 (p, J=6 Hz, 1H), 4.36 (dd, J=11 and 6 Hz, 1H), 4.44 (dd, J=11 and 5 Hz, 1H), 6.56 (d, J=3 Hz, 1H), 8.75 (brs, 1H).

### Example 85

### 1-((Isopropoxycarbonyl)oxy)ethyl 3-chloro-4-(dodecyloxy)-1H-pyrrole-2-carboxylate

Obtained as a pale oil (53%) from 3-chloro-4-(dodecyloxy)-1H-pyrrole-2-carboxylic acid (Example 80) and 1-chloroethyl isopropyl carbonate following the experimental procedure described in Example 8 followed by purification of the crude product by flash chromatography (hexanes/DCM).

MS (m/z): 477 [M+17]⁺

¹H NMR δ (400 MHz, CDCl₃) 0.83-0.93 (m, 59H), 1.20-1.52 (m, 24H), 1.63 (d, J=5.4 Hz, 3H), 1.70-1.83 (m, 2H), 3.89 (t, J=6.7 Hz, 2H), 4.90 (p, J=6.3 Hz, 1H), 6.55 (d, J=3.5 Hz, 1H), 6.97 (q, J=5.4 Hz, 1H), 8.59 (s, 1H).

### Example 86

### 4-Oxo-3,5,8,11-tetraoxatridecan-2-yl 3-chloro-4-(dodecyloxy)-1H-pyrrole-2-carboxylate

Obtained as a yellow oil (12%) from 3-chloro-4-(dodecyloxy)-1H-pyrrole-2-carboxylic acid (Example 80) and 1-chloroethyl (2-(2-ethoxyethoxy)ethyl) carbonate (Intermediate 7) following the experimental procedure described in Example 8 followed by purification of the crude product by reverse phase chromatography (water/ACN both containing 0.01% of formic acid).

MS (m/z): 551 [M+17]⁺

¹H-NMR δ (400 MHz, CDCl₃): 0.83-0.91 (m, 3H), 1.21 (t, J=7.0 Hz, 3H), 1.28 (s, 18H), 1.39-1.47 (m, 2H), 1.63 (d, J=5 Hz, 3H), 1.71-1.80 (m, 2H), 3.53 (q, J=7 Hz, 2H), 3.57-3.60 (m, 2H), 3.62-3.66 (m, 2H), 3.71-3.75 (m, 2H), 3.89 (t, J=7 Hz, 2H), 4.30-4.35 (m, 2H), 6.54 (d, J=3 Hz, 1H), 6.97 (q, J=5 Hz, 1H), 8.71 (s, 1H).

### Example 87

### 1-(((3-Hydroxypropoxy)carbonyl)oxy)ethyl 3-chloro-4-(dodecyloxy)-1H-pyrrole-2-carboxylate

To a suspension of 3-chloro-4-(dodecyloxy)-1H-pyrrole-2-carboxylic acid (Example 80, 127 mg, 0.385 mmol) and triethylamine (161 μL, 1.16 mmol) in ACN (2.5 mL) was added 3-(benzyloxy)propyl (1-chloroethyl) carbonate (Intermediate 12, 158 mg, 0.58 mmol) and the mixture was heated at 100° C. for 20 h. Solvent was then removed and purification of the resulting residue by flash chromatography (hexanes/diethyl ether) gave 1-(((3-(benzyloxy)propoxy) carbonyl)oxy)ethyl 3-chloro-4-(dodecyloxy)-1H-pyrrole-2-carboxylate (100 mg, 46%) as a clear oil.

MS (m/z): 583/585 [M+17/M+19]⁺.

¹H-NMR δ (400 MHz, CDCl₃): 0.88 (t, J=7 Hz, 3H), 1.23-1.36 (m, 20H), 1.38-1.47 (m, 2H), 1.63 (d, J=5 Hz, 3H), 1.71-1.80 (m, 2H), 1.98 (p, J=6 Hz, 2H), 3.56 (t, J=6 Hz, 2H), 3.88 (t, J=6 Hz, 2H), 4.30 (t, J=6 Hz, 2H), 4.49 (s, 2H), 6.53 (d, J=4 Hz, 1H), 6.59 (q, J=5 Hz, 1H), 7.23-7.36 (m, 5H), 8.60 (brs, 1H).

To a solution of 1-(((3-(benzyloxy)propoxy)carbonyl)oxy)ethyl 3-chloro-4-(dodecyloxy)-1H-pyrrole-2-carboxylate (95 mg, 0.168 mmol) in THF (5 mL) was added 10% Pd—C (18 mg, 0.017 mmol) and the resulting suspension was stirred under a hydrogen atmosphere for 2 h. The reaction mixture was then filtered over a Celite® pad and the solvent was evaporated. Purification of the residue by reverse phase chromatography (water/ACN both containing 0.01% of formic acid) gave the title compound (60 mg, 75%) as a clear oil.

MS (m/z): 493/495 [M+17/M+19]⁺.

¹H-NMR δ (400 MHz, CDCl₃): 0.88 (t, J=7 Hz, 3H), 1.20-1.38 (m, 18H), 1.39-1.46 (m, 2H), 1.63 (d, J=5 Hz, 3H), 1.76 (p, J=7 Hz, 2H), 1.92 (p, J=6 Hz, 2H), 3.74 (t, J=6 Hz, 2H), 3.89 (t, J=7 Hz, 2H), 4.28-4.40 (m, 2H), 6.56 (d, J=3 Hz, 1H), 6.96 (q, J=5 Hz, 1H), 8.70 (brs, 1H).

### Example 88

### (5-Methyl-2-oxo-1,3-dioxol-4-yl)methyl 3-chloro-4-(dodecyloxy)-1H-pyrrole-2-carboxylate

A mixture of 3-chloro-4-(dodecyloxy)-1H-pyrrole-2-carboxylic acid (Example 80, 100 mg, 0.30 mmol), 4-(bromomethyl)-5-methyl-1,3-dioxol-2-one (70 mg, 0.36 mmol) and potassium carbonate (63 mg, 0.45 mmol) in DMF (2 mL) was stirred at room temperature for 16 h. Water and DCM were added, the organic layer was separated and the aqueous layer was extracted with DCM (×1). The combined organic extracts were dried over magnesium sulfate, filtered and the solvent was evaporated. The residue was purified by flash chromatography (hexanes/EtOAc) to yield the title compound (69 mg, 51%).

¹H NMR δ (400 MHz, CDCl₃) 0.75-1.02 (m, 3H), 0.98-1.52 (m, 18H), 1.64-1.88 (m, 2H), 2.22 (s, 3H), 3.90 (t, J=6.6 Hz, 2H), 5.05 (s, 2H), 6.56 (d, J=3.5 Hz, 1H), 8.60 (s, 1H).

### Example 89

### 3-Chloro-4-(tridecyloxy)-1H-pyrrole-2-carboxylic acid

Obtained as a light grey solid (37%) from methyl 3-chloro-4-(tridecyloxy)-1H-pyrrole-2-carboxylate (Intermediate 38) following the experimental procedure as described in Example 21.

MS (m/z): 344/346 [M+1/M+3]⁺.

¹H-NMR δ (400 MHz, CDCl₃): 0.89 (t, J=7 Hz, 3H), 1.22-1.53 (m, 20H), 1.68-1.77 (m, 2H), 3.89 (t, J=6 Hz, 2H), 6.60 (br s, 1H).

### Example 90

### 3-Chloro-4-(tetradecyloxy)-1H-pyrrole-2-carboxylic acid

Obtained as a white solid (85%) from methyl 3-chloro-4-(tetradecyloxy)-1H-pyrrole-2-carboxylate (Intermediate 39) following the experimental procedure as described in Example 12.

MS (m/z): 358/360 [M+1/M+3]⁺

1H-NMR δ (400 MHz, DMSO-d6): 0.81-0.89 (m, 3H), 1.18-1.30 (m, 20H), 1.32-1.42 (m, 2H), 1.64 (p, J=6 Hz, 2H), 3.84 (t, J=6 Hz, 2H), 6.70 (s, 1H), 11.50 (s, 1H).

### Example 91

### 3-Fluoro-4-pentadecanoyl-1H-pyrrole-2-carboxylic acid

Obtained as an off-white solid (63%) from ethyl 3-fluoro-4-pentadecanoyl-1H-pyrrole-2-carboxylate (Intermediate 14a) following the experimental procedure described in Example 21.

MS (m/z): 352 [M−1]⁺.

¹H-NMR δ (400 MHz, DMSO-d6): 0.81-0.88 (m, 3H), 1.17-1.31 (m, 18H), 1.45-1.58 (m, 4H), 2.18 (t, J=7.3 Hz, 2H), 2.69 (t, J=7.3 Hz, 2H), 7.46 (d, J=3.9 Hz, 1H).

### Example 92

### 4-(12-Ethoxydodecyl)-3-fluoro-1H-pyrrole-2-carboxylic acid

Obtained as a white solid (25%) from ethyl 4-(12-bromododecyl)-3-fluoro-1H-pyrrole-2-carboxylate (Intermediate 40c) following the experimental procedure described in Example 12 followed by purification of the crude product by reverse phase chromatography (water/ACN both containing 0.01% of formic acid).

MS (m/z): 342 [M+1]⁺

¹H-NMR δ (400 MHz, MeOD): 1.18 (t, J=7 Hz, 3H), 1.31 (s, 16H), 1.63-1.49 (m, 4H), 2.40 (t, J=7 Hz, 2H), 3.56-3.38 (m, 4H), 6.58 (d, J=5 Hz, 1H).

### Example 93

### 3-Fluoro-4-(2-fluorotridecyl)-1H-pyrrole-2-carboxylic acid

Obtained as a white solid (49%) from ethyl 3-fluoro-4-(2-fluorotridecyl)-1H-pyrrole-2-carboxylate (Intermediate 41c) following the experimental procedure described in Example 12 followed by purification of the crude product by flash chromatography (diethyl ether/methanol).

MS (m/z): 330 [M+1]⁺

1H-NMR δ (400 MHz, CD₃OD): 0.90 (t, J=7 Hz, 3H), 1.29 (s, 18H), 1.51-1.66 (m, 2H), 2.71 (dd, J=22 and 6 Hz, 2H), 4.47-4.72 (m, 1H), 6.68 (d, J=5 Hz, 1H).

### Example 94

### 4-(2,2-Difluorotridecyl)-3-fluoro-1H-pyrrole-2-carboxylic acid

To a cooled (0° C.) solution of ethyl 4-(2,2-difluorotridecanoyl)-3-fluoro-1H-pyrrole-2-carboxylate (Intermediate 42, 88 mg, 0.23 mmol) in TFA (1.5 mL) was added dropwise triethylsilane (0.11 mL, 0.68 mmol) and the mixture was stirred at room temperature for 2 h. Trifluoroacetic acid was removed under reduced pressure and the crude was partitioned between DCM and aqueous saturated sodium hydrogen carbonate solution. The organic phase was separated, washed with aqueous saturated sodium hydrogen carbonate solution, water and brine, dried over magnesium sulfate, filtered and the solvent evaporated to dryness. The resulting brown semisolid was dissolved in ethanol (1 mL), aqueous 4M sodium hydroxide solution (0.14 mL, 0.56 mmol) was added and the mixture was heated at reflux for 1 h. The solvent was removed in vacuo, water was added and the pH of the solution was adjusted to 2-3 by addition of 1N hydrochloric acid solution. The reaction mixture was then extracted with EtOAc (×3). The combined organic extracts were washed with water and brine, dried over magnesium sulfate, filtered and the solvent evaporated to dryness. The residue was purified by reverse phase chromatography (water/ACN both containing 0.01% of formic acid) to yield the title product (6 mg, 9%) as a white solid.

MS (m/z): 348 [M+1]⁺

¹H-NMR δ (400 MHz, CD₃OD): 1.01-0.82 (m, 3H), 1.29 (s, 16H), 1.56-1.43 (m, 2H), 1.80 (dq, J=17 and 8 Hz, 2H), 2.97 (t, J=16 Hz, 2H), 6.70 (d, J=5 Hz, 1H).

### Example 95

### 4-(3,3-Dimethyldodecyl)-3-fluoro-1H-pyrrole-2-carboxylic acid

Obtained as a brown solid (88%) from ethyl 4-(3,3-dimethyldodecyl)-3-fluoro-1H-pyrrole-2-carboxylate (Intermediate 43d) following the experimental procedure described in Example 12.

MS (m/z): 326 [M+1]⁺

¹H-NMR δ (400 MHz, DMSO-d6): 0.79-0.88 (m, 9H), 1.21 (m, 16H), 1.32-1.41 (m, 2H), 2.20-2.28 (m, 2H), 6.51-6.74 (m, 1H), 11.20 (s, 1H).

### Example 96

### 4-((2,2-Dimethyltridecyl)oxy)-3-fluoro-1H-pyrrole-2-carboxylic acid

Obtained as an off-white solid (14%) from ethyl 4-((2,2-dimethyltridecyl)oxy)-3-fluoro-1H-pyrrole-2-carboxylate (Intermediate 44e) following the experimental procedure described in Example 12 followed by purification of the crude product by preparative HPLC-MS (gradient from water to ACN/methanol 1:1).

¹H-NMR δ (400 MHz, CDCl₃): 0.84-0.91 (m, 3H), 0.96 (s, 6H), 1.22-1.33 (m, 20H), 3.56 (s, 2H), 6.46 (s, 1H), 8.40 (s, 1H).

### Example 97

### 4-((2,2-Difluorotetradecyl)oxy)-3-fluoro-1H-pyrrole-2-carboxylic acid

Obtained as an off-white solid (82%) from ethyl 4-((2,2-difluorotetradecyl)oxy)-3-fluoro-1H-pyrrole-2-carboxylate (Intermediate 45c) following the experimental procedure described in Example 21.

¹H-NMR δ (400 MHz, CDCl₃): 0.81-0.89 (m, 3H), 1.22-1.32 (m, 18H), 1.37-1.49 (m, 2H), 1.85-2.04 (m, 2H), 4.12 (t, J=13.0 Hz, 2H), 6.62 (s, 1H), 10.97 (s, 1H).

### Example 98

### 4-((2,2-Difluoroundecyl)oxy)-3-fluoro-1H-pyrrole-2-carboxylic acid

Obtained as a white solid (52%) from ethyl 4-((2,2-difluoroundecyl)oxy)-3-fluoro-1H-pyrrole-2-carboxylate (Intermediate 46c) following the experimental procedure described in Example 12. The crude product was triturated with hexane, filtered and dryed to give the title compound.

¹H-NMR δ (400 MHz, CDCl₃): 0.79-0.89 (m, 3H), 1.20-1.35 (m, 12H), 1.39-1.47 (m, 2H), 1.86-2.04 (m, 2H), 4.15 (t, J=12.9 Hz, 2H), 6.76 (t, J=4.0 Hz, 1H), 11.26 (s, 1H), 12.59 (s, 1H).

### Example 99

### 3-Chloro-4-((2-fluorotetradecyl)oxy)-1H-pyrrole-2-carboxylic acid

Obtained as a yellow solid (49%) from methyl 3-chloro-4-((2-fluorotetradecyl) oxy)-1H-pyrrole-2-carboxylate (Intermediate 47b) following the experimental procedure described in Example 12.

MS (m/z): 374 [M−1]⁺

¹H NMR δ (400 MHz, MeOD): 0.85-0.93 (m, 3H), 1.21-1.55 (m, 20H), 1.61-1.80 (m, 2H), 3.93-3.98 (m, 1H), 3.98-4.07 (m, 1H), 4.62-4.82 (m, 1H), 6.63 (s, 1H).

### Example 100

### 3-Chloro-4-((9-ethoxynonyl)oxy)-1H-pyrrole-2-carboxylic acid

Obtained as a white solid (20%) from ethyl 3-chloro-4-(9-ethoxynonoxy)-1H-pyrrole-2-carboxylate (Intermediate 48c) following the experimental procedure described in Example 12 followed by purification of the crude product by reverse phase chromatography (water/ACN both containing 0.01% of formic acid) and flash chromatography (DCM/methanol).

MS (m/z): 332 [M+1]⁺

¹H NMR δ (400 MHz, CDCl₃): 1.20 (t, J=7.0 Hz, 3H), 1.25-1.39 (m, 8H), 1.39-1.50 (m, 2H), 1.57 (p, J=6.8 Hz, 2H), 1.70-1.82 (m, 2H), 3.41 (t, J=6.8 Hz, 2H), 3.44-3.53 (m, 2H), 3.91 (t, J=6.6 Hz, 2H), 6.60 (d, J=3.4 Hz, 1H), 8.83 (s, 1H).

### Example 101

### 3-Methyl-4-tridecyl-1H-pyrrole-2-carboxylic acid

Obtained as a solid (87%) from ethyl 3-methyl-4-tridecyl-1H-pyrrole-2-carboxylate (Intermediate 49b) following the experimental procedure described in Example 12.

MS (m/z): 308 [M+1]⁺

¹H NMR δ (400 MHz, CDCl₃): 0.86 (t, J=7 Hz, 3H), 1.24 (s, 20H), 1.50 (p, J=7 Hz, 2H), 2.28 (s, 3H), 2.32-2.46 (m, 2H), 6.66 (d, J=3 Hz, 1H).

### Example 102

### 4-(2,2-Dimethyldodecyl)-3-fluoro-1H-pyrrole-2-carboxylic acid

Obtained as a white solid (98%) from ethyl 4-(2,2-dimethyldodecyl)-3-fluoro-1H-pyrrole-2-carboxylate (Intermediate 50c) following the experimental procedure described in Example 1 using methanol as solvent for the reaction and DCM as solvent for the final extraction.

MS (m/z): 326 [M+1]⁺.

¹H NMR δ (400 MHz, CDCl₃): 0.85 (s, 6H), 0.86-0.94 (m, 3H), 1.22-1.33 (m, 18H), 2.31 (s, 2H), 6.58-6.64 (m, 1H), 8.55 (s, 1H).

### Example 103

### 2,2,2-Trifluoroethyl 3-fluoro-4-(undecyloxy)-1H-pyrrole-2-carboxylate

Obtained as a white solid (30%) from 3-fluoro-4-(undecyloxy)-1H-pyrrole-2-carboxylic acid (Example 72) and 2,2,2-trifluoroethan-1-ol following the experimental procedure described in Example 25 followed by purification of the crude product by flash chromatography (hexanes/DCM).

¹H NMR δ (400 MHz, CDCl₃): 0.83-0.91 (m, 3H), 1.22-1.48 (m, 16H), 1.66-1.82 (m, 2H), 3.93 (t, J=6.6 Hz, 2H), 4.65 (q, J=8.4 Hz, 2H), 6.54 (t, J=4.1 Hz, 1H), 8.10 (s, 1H).

### Example 104

### 2-(2-Ethoxyethoxy)ethyl 3-fluoro-4-(undecyloxy)-1H-pyrrole-2-carboxylate

Obtained as a solid (18%) from 3-fluoro-4-(undecyloxy)-1H-pyrrole-2-carboxylic acid (Example 72) and 2-(2-ethoxyethoxy)ethanol following the experimental procedure described in Example 25 followed by purification of the crude product by flash chromatography (hexanes/EtOAc).

MS (m/z): 416 [M+1]⁺.

¹H-NMR δ (400 MHz, CDCl₃): 0.88 (t, J=7 Hz, 3H), 1.21 (t, J=7 Hz, 3H), 1.23-1.36 (m, 14H), 1.37-1.45 (m, 2H), 1.69-1.76 (m, 2H), 3.53 (q, J=7 Hz, 2H), 3.59-3.62 (m, 2H), 3.69-3.71 (m, 2H), 3.79-3.82 (m, 2H), 3.91 (t, J=7 Hz, 2H); 4.42-4.45 (m, 2H), 6.43-6.46 (m, 1H), 8.29 (brs, 1H).

### Example 105

### 1-((Isopropoxycarbonyl)oxy)ethyl 3-fluoro-4-(undecyloxy)-1H-pyrrole-2-carboxylate

Obtained as a white solid (50%) from 3-fluoro-4-(undecyloxy)-1H-pyrrole-2-carboxylic acid (Example 72) and 1-chloroethyl isopropyl carbonate following the experimental procedure described in Example 8 followed by purification of the crude product by flash chromatography (hexanes/diethyl ether).

MS (m/z): 430 [M+1]⁺. 1H-NMR δ (400 MHz, CDCl₃): 0.84-0.92 (m, 3H), 1.24-1.33 (m, 20H), 1.36-1.46 (m, 2H), 1.61 (d, J=5 Hz, 3H), 1.68-1.76 (m, 2H), 3.91 (t, J=7 Hz, 2H), 4.90 (hept, J=6 Hz, 1H), 6.50 (t, J=4 Hz, 2H), 6.97 (q, J=5 Hz, 1H), 8.13 (s, 1H).

### Example 106

### 1-(((2-Methoxyethoxy)carbonyl)oxy)ethyl 3-fluoro-4-(undecyloxy)-1H-pyrrole-2-carboxylate

Obtained as a white solid (23%) from 3-fluoro-4-(undecyloxy)-1H-pyrrole-2-carboxylic acid (Example 72) and 1-chloroethyl (2-methoxyethyl) carbonate (Intermediate 6) following the experimental procedure described in Example 8 followed by purification of the crude product by flash chromatography (using hexanes/DCM and hexanes/diethyl ether as eluents).

MS (m/z): 463 [M+18]⁺.

¹H NMR δ (400 MHz, CDCl₃): 0.83-0.93 (m, 3H), 1.17-1.47 (m, 16H), 1.62 (d, J=5.4 Hz, 3H), 1.66-1.78 (m, 2H), 3.38 (s, 3H), 3.61 (t, J=4.7 Hz, 2H), 3.91 (t, J=6.6 Hz, 2H), 4.20-4.38 (m, 2H), 6.50 (t, J=4.1 Hz, 1H), 6.98 (q, J=5.4 Hz, 1H), 8.23 (s, 1H).

### Example 107

### 4-Oxo-3,5,8,11-tetraoxatridecan-2-yl 3-fluoro-4-(undecyloxy)-1H-pyrrole-2-carboxylate

Obtained as a colourless oil (14%) from 3-fluoro-4-(undecyloxy)-1H-pyrrole-2-carboxylic acid (Example 72) and 1-chloroethyl (2-(2-ethoxyethoxy)ethyl) carbonate (Intermediate 7) following the experimental procedure described in Example 8 followed by purification of the crude product by flash chromatography (hexanes/diethyl ether) and reverse phase chromatography (water/AON both with 0.5% of formic acid).

MS (m/z): 504 [M+1]⁺ and 521 [M+17]⁺.

¹H-NMR δ (400 MHz, CDCl₃): 0.85-0.92 (m, 3H), 1.21 (t, J=7 Hz, 3H), 1.24-1.35 (m, 14H), 1.37-1.46 (m, 2H), 1.62 (d, J=5 Hz, 3H), 1.68-1.77 (m, 2H), 3.53 (q, J=7 Hz, 2H), 3.57-3.60 (m, 2H), 3.62-3.66 (m, 2H), 3.71-3.75 (m, 2H), 3.91 (t, J=7 Hz, 2H), 4.32 (ddd, J=6, 4 and 1 Hz, 2H), 6.49 (t, J=4 Hz, 1H), 6.97 (q, J=5 Hz, 1H), 8.18 (s, 1H).

### Example 108

### 2,2,2-Trifluoroethyl 3-chloro-4-tridecyl-1H-pyrrole-2-carboxylate

Obtained as a white solid (29%) from 3-chloro-4-tridecyl-1H-pyrrole-2-carboxylic acid (Example 61) and 2,2,2-trifluoroethan-1-ol following the experimental procedure described in Example 25 followed by purification of the crude product by flash chromatography (using hexanes/DCM and hexanes/EtOAc as eluents).

¹H NMR δ (400 MHz, CDCl₃): 0.83-0.92 (m, 3H), 1.22-1.39 (m, 20H), 1.53 (d, J=8.9 Hz, 2H), 2.41-2.49 (m, 2H), 4.66 (q, J=8.4 Hz, 2H), 6.77 (d, J=3.3 Hz, 1H), 8.86 (s, 1H).

### Example 109

### 2-(2-Ethoxyethoxy)ethyl 3-chloro-4-tridecyl-1H-pyrrole-2-carboxylate

Obtained (13%) from 3-chloro-4-tridecyl-1H-pyrrole-2-carboxylic acid (Example 61) and 2-(2-ethoxyethoxy)ethanol following the experimental procedure described in Example 25 followed by purification of the crude product by flash chromatography (hexanes/EtOAc).

MS (m/z): 444/446 [M+1/M+3]⁺.

¹H-NMR δ (400 MHz, CDCl₃): 0.88 (t, J=7 Hz, 3H), 1.21 (t, J=7 Hz, 3H), 1.24-1.36 (m, 20H), 1.54 (p, J=7 Hz, 2H), 2.44 (t, J=8 Hz, 2H), 3.54 (q, J=7 Hz, 2H), 3.59-3.62 (m, 2H), 3.69-3.71 (m, 2H), 4.42-4.45 (m, 2H), 6.68 (d, J=3 Hz, 1H), 9.09 (brs, 1H).

### Example 110

### 1-((Isopropoxycarbonyl)oxy)ethyl 3-chloro-4-tridecyl-1H-pyrrole-2-carboxylate

Obtained as a colourless oil (55%) from 3-chloro-4-tridecyl-1H-pyrrole-2-carboxylic acid (Example 61) and 1-chloroethyl isopropyl carbonate following the experimental procedure described in Example 8 followed by purification of the crude product by flash chromatography (hexanes/diethyl ether).

MS (m/z): 458/460 [M+1]⁺.

1H-NMR δ (400 MHz, CDCl₃): 0.85-0.91 (m, 3H), 1.22-1.35 (m, 26H), 1.53 (m, 2H), 1.63 (d, J=5 Hz, 3H), 2.40-2.47 (m, 2H), 4.90 (hept, J=6 Hz, 1H), 6.72 (d, J=3 Hz, 1H), 6.98 (q, J=5 Hz, 1H), 8.86 (s, 1H).

### Example 111

### 1-(((2-methoxyethoxy)carbonyl)oxy)ethyl 3-chloro-4-tridecyl-1H-pyrrole-2-carboxylate

Obtained as a colourless oil (29%) from 3-chloro-4-tridecyl-1H-pyrrole-2-carboxylic acid (Example 61) and 1-chloroethyl (2-methoxyethyl) carbonate (Intermediate 6) following the experimental procedure described in Example 8 followed by purification of the crude product by flash chromatography (using hexanes/EtOAc and hexanes/DCM as eluents).

MS (m/z): 491 [M+17]⁺.

¹H NMR δ (400 MHz, CDCl₃): 0.88 (t, J=6.8 Hz, 3H), 1.20-1.43 (m, 20H), 1.54 (p, J=7.3 Hz, 2H), 1.63 (d, J=5.4 Hz, 3H), 2.43 (t, J=7.6 Hz, 2H), 3.38 (s, 3H), 3.61 (t, J=4.7 Hz, 2H), 4.17-4.40 (m, 2H), 6.72 (d, J=3.2 Hz, 1H), 6.99 (q, J=5.4 Hz, 1H), 8.89 (s, 1H).

### Example 112

### 4-Oxo-3,5,8,11-tetraoxatridecan-2-yl 3-chloro-4-tridecyl-1H-pyrrole-2-carboxylate

Obtained as a colourless oil (15%) from 3-chloro-4-tridecyl-1H-pyrrole-2-carboxylic acid (Example 61) and 1-chloroethyl (2-(2-ethoxyethoxy)ethyl) carbonate (Intermediate 7) following the experimental procedure described in Example 8 followed by purification of the crude product by reverse phase chromatography.

MS (m/z): 532 [M+1]⁺ and 549 [M+17]⁺. 1H-NMR δ (400 MHz, CDCl₃): 0.85-0.91 (m, 3H), 1.21 (t, J=7 Hz, 3H), 1.26-1.36 (m, 20H), 1.55 (d, J=7 Hz, 2H), 1.63 (d, J=5 Hz, 3H), 2.40-2.46 (m, 2H), 3.48-3.56 (m, 2H), 3.56-3.60 (m, 2H), 3.62-3.66 (m, 2H), 3.71-3.74 (m, 2H), 4.30-4.34 (m, 2H), 6.69-6.74 (m, 1H), 6.98 (q, J=5 Hz, 1H), 8.96 (s, 1H).

### Example 113

### 2,2,2-Trifluoroethyl 3-chloro-5-dodecyl-1H-pyrrole-2-carboxylate

Obtained as a white solid (55%) from 3-chloro-5-dodecyl-1H-pyrrole-2-carboxylic acid (Example 65) and 2,2,2-trifluoroethan-1-ol following the experimental procedure described in Example 25 followed by purification of the crude product by flash chromatography (hexanes/DCM).

¹H NMR δ (400 MHz, CDCl₃): 0.85-0.91 (m, 3H), 1.23-1.39 (m, 18H), 1.61 (p, J=7.7 Hz, 2H), 2.52-2.63 (m, 2H), 4.65 (q, J=8.4 Hz, 2H), 6.03 (d, J=3.1 Hz, 1H), 8.67 (s, 1H).

### Example 114

### 2-(2-Ethoxyethoxy)ethyl 3-chloro-5-dodecyl-1H-pyrrole-2-carboxylate

Obtained (33%) from 3-chloro-5-dodecyl-1H-pyrrole-2-carboxylic acid (Example 65) and 2-(2-ethoxyethoxy)ethanol following the experimental procedure described in Example 25 followed by purification of the crude product by flash chromatography (hexanes/EtOAc).

MS (m/z): 430/432 [M+1/M+3]⁺.

¹H-NMR δ (400 MHz, CDCl₃): 0.88 (t, J=7 Hz, 3H), 1.21 (t, J=7 Hz, 3H), 1.22-1.36 (m, 18H), 1.60 (p, J=7 Hz, 2H), 2.54 (t, J=8 Hz, 2H), 3.53 (q, J=7 Hz, 2H), 3.58-3.63 (m, 2H), 3.68-3.72 (m, 2H), 3.79-3.84 (m, 2H), 4.40-4.45 (m, 2H), 5.97 (d, J=3 Hz, 1H), 8.89 (br s, 1H).

### Example 115

### 1-((Isopropoxycarbonyl)oxy)ethyl 3-chloro-5-dodecyl-1H-pyrrole-2-carboxylate

Obtained as a brown oil (56%) from 3-chloro-5-dodecyl-1H-pyrrole-2-carboxylic acid (Example 65) and 1-chloroethyl isopropyl carbonate following the experimental procedure described in Example 8 followed by purification of the crude product by flash chromatography (hexanes/diethyl ether).

MS (m/z): 444 [M]⁺.

¹H NMR δ (400 MHz, CDCl₃): 0.86-0.93 (m, 3H), 1.23-1.28 (m, 18H), 1.30 (dd, J=6.2, 1.4 Hz, 6H), 1.56-1.61 (m, 2H), 1.63 (d, J=5.4 Hz, 3H), 2.54 (t, J=7.6 Hz, 2H), 4.82-5.00 (m, 1H), 5.98 (d, J=3.1 Hz, 1H), 6.97 (q, J=5.4 Hz, 1H), 8.67 (s, 1H).

### Example 116

### 1-(((2-Methoxyethoxy)carbonyl)oxy)ethyl 3-chloro-5-dodecyl-1H-pyrrole-2-carboxylate

Obtained (25%) from 3-chloro-5-dodecyl-1H-pyrrole-2-carboxylic acid (Example 65) and 1-chloroethyl (2-methoxyethyl) carbonate (Intermediate 6) following the experimental procedure described in Example 8 followed by purification of the crude product by flash chromatography (using hexanes/diethyl ether and hexanes/DCM as eluents).

MS (m/z): 477,479 [M+17,M+19]⁺.

¹H NMR δ (400 MHz, CDCl₃): 0.82-0.95 (m, 3H), 1.21-1.38 (m, 18H), 1.53-1.61 (m, 2H), 1.63 (d, J=5.4 Hz, 3H), 2.55 (t, J=7.6 Hz, 2H), 3.37 (s, 3H), 3.61 (t, J=4.7 Hz, 2H), 4.22-4.37 (m, 2H), 5.95-6.00 (m, 1H), 6.97 (q, J=5.4 Hz, 1H), 8.83 (s, 1H).

### Example 117

### 4-Oxo-3,5,8,11-tetraoxatridecan-2-yl 3-chloro-5-dodecyl-1H-pyrrole-2-carboxylate

Obtained as an oil (27%) from 3-chloro-5-dodecyl-1H-pyrrole-2-carboxylic acid (Example 65) and 1-chloroethyl (2-(2-ethoxyethoxy)ethyl) carbonate (Intermediate 7) following the experimental procedure described in Example 8 followed by purification of the crude product by flash chromatography (using DCM/methanol and hexanes/diethyl ether as eluents).

MS (m/z): 535,537 [M+17, M+19]⁺.

¹H NMR δ (400 MHz, CDCl₃): 0.84-0.92 (m, 3H), 1.21 (t, J=7.0 Hz, 3H), 1.24-1.41 (m, 18H), 1.56-1.61 (m, 2H), 1.63 (d, J=5.4 Hz, 3H), 2.55 (t, J=7.7 Hz, 2H), 3.52 (q, J=7.0 Hz, 3H), 3.56-3.66 (m, 4H), 3.71-3.77 (m, 2H), 4.28-4.39 (m, 2H), 5.99 (d, J=3.1 Hz, 1H), 6.97 (q, J=5.4 Hz, 1H), 8.72 (s, 1H).

### Example 118

### 2,3-Dihydroxypropyl 3-chloro-5-dodecyl-1H-pyrrole-2-carboxylate

Obtained (27%) from 3-chloro-5-dodecyl-1H-pyrrole-2-carboxylic acid (Example 65) and propane-1,2,3-triol (10 equivalents) following the experimental procedure described in Example 25 followed by purification of the crude product by flash chromatography (hexanes/EtOAc).

MS (m/z): 388/390 [M+1/M+3]⁺.

¹H-NMR δ (400 MHz, CDCl₃): 0.88 (t, J=7 Hz, 3H), 1.17-1.36 (m, 18H), 1.60 (p, J=7 Hz, 2H), 2.55 (t, J=8 Hz, 1H), 3.72 (dd, J=11 and 6 Hz, 1H), 3.78 (dd, J=11 and 4 Hz, 1H), 3.93 (d, J=4 Hz, 1H), 4.00-4.07 (m, 1H), 4.34 (dd, J=11 and 6 Hz, 1H), 4.42 (dd, J=11 and 5 Hz, 1H), 5.99 (d, J=3 Hz, 1H), 8.88 (brs, 1H).

### Example 119

### 3-Fluoro-5-undecyl-1H-pyrrole-2-carboxylic acid

Obtained as a solid (75%) from ethyl 3-fluoro-5-undecyl-1H-pyrrole-2-carboxylate (Intermediate 51) following the experimental procedure described in Example 21.

MS (m/z): 284 [M+1]⁺.

¹H-NMR δ (400 MHz, CDCl₃): 0.84-0.92 (m, 3H), 1.21-1.39 (m, 16H), 1.60 (q, J=7.2 Hz, 2H), 2.55 (t, J=7.7 Hz, 2H), 5.78 (d, J=3.1 Hz, 1H), 8.38 (s, 1H).

### Example 120

### 3-Fluoro-5-tridecyl-1H-pyrrole-2-carboxylic acid

Obtained as a solid (75%) from ethyl 3-fluoro-5-tridecyl-1H-pyrrole-2-carboxylate (Intermediate 52b) following the experimental procedure described in Example 21.

MS (m/z): 312 [M+1]⁺.

¹H-NMR δ (400 MHz, CDCl₃): 0.81-0.92 (m, 3H), 1.21-1.40 (m, 20H), 1.53-1.69 (m, 2H), 2.55 (t, J=7.7 Hz, 2H), 5.74-5.81 (m, 1H), 8.34 (s, 1H).

### Example 121

### 3-Fluoro-5-tetradecyl-1H-pyrrole-2-carboxylic acid

Obtained as a light brown solid (63%) from ethyl 3-fluoro-5-tetradecyl-1H-pyrrole-2-carboxylate (Intermediate 53) following the experimental procedure described in Example 64.

MS (m/z): 326 [M+1]⁺.

¹H-NMR δ (400 MHz, CDCl₃): 0.88 (t, J=7 Hz, 3H), 1.19-1.36 (m, 22H), 1.57-1.64 (m, 2H), 2.55 (t, J=8 Hz, 2H), 5.77 (d, J=3 Hz, 1H), 8.53 (brs, 1H).

### Example 122

### 3-Fluoro-5-pentadecyl-1H-pyrrole-2-carboxylic acid

Obtained as an off white solid (66%) from ethyl 3-fluoro-5-pentadecyl-1H-pyrrole-2-carboxylate (Intermediate 54) following the experimental procedure described in Example 56.

MS (m/z): 340 [M+1]⁺.

¹H-NMR δ (400 MHz, CDCl₃): 0.88 (t, J=7 Hz, 3H), 1.20-1.34 (m, 24H), 1.51-1.62 (m, 2H), 2.45-2.54 (m, 2H), 5.71 (brs, 1H), 8.55 (brs, 1H).

### Example 123

### 3-Fluoro-5-hexadecyl-1H-pyrrole-2-carboxylic acid

Obtained as a white solid (79%) from ethyl 3-fluoro-5-hexadecyl-1H-pyrrole-2-carboxylate (Intermediate 55) following the experimental procedure described in Example 21 followed by purification of the crude product by reverse phase chromatography (water/methanol).

MS (m/z): 354 [M+1]⁺.

¹H NMR δ (400 MHz, DMSO-d6): 0.83 (t, J=7.0 Hz, 3H), 1.14-1.30 (m, 26H), 1.40-1.48 (m, 2H), 2.30-2.39 (m, 2H), 5.40 (s, 1H), 9.90 (s, 1H).

### Example 124

### 3-Fluoro-5-heptadecyl-1H-pyrrole-2-carboxylic acid

Obtained as a white solid (81%) from ethyl 3-fluoro-5-heptadecyl-1H-pyrrole-2-carboxylate (Intermediate 56) following the experimental procedure described in Example 21 followed by purification of the crude product by reverse phase chromatography (water/methanol).

MS (m/z): 368 [M+1]⁺.

¹H NMR δ (400 MHz, DMSO-d6): 0.85 (t, J=6.8 Hz, 3H), 1.19-1.28 (m, 28H), 1.45-1.57 (m, 2H), 2.47 (d, J=7.6 Hz, 2H), 5.75 (d, J=2.6 Hz, 1H), 11.21 (s, 1H).

### Example 125

### 3-Fluoro-5-octadecyl-1H-pyrrole-2-carboxylic acid

Obtained as a white solid (14%) from ethyl 3-fluoro-5-octadecyl-1H-pyrrole-2-carboxylate (Intermediate 57) following the experimental procedure described in Example 21 followed by purification of the crude product by reverse phase chromatography (water/methanol).

MS (m/z): 382 [M+1]⁺.

¹H NMR δ (400 MHz, CDCl₃): 0.88 (t, J=6.8 Hz, 3H), 1.19-1.35 (m, 30H), 1.53-1.66 (m, 2H), 2.55 (t, J=7.6 Hz, 2H), 5.78 (d, J=3.1 Hz, 1H), 8.37 (s, 1H).

### Example 126

### 3-Fluoro-5-nonadecyl-1H-pyrrole-2-carboxylic acid

Obtained as a white solid (67%) from ethyl 3-fluoro-5-nonadecyl-1H-pyrrole-2-carboxylate (Intermediate 58) following the experimental procedure described in Example 56.

MS (m/z): 396 [M+1]⁺.

¹H NMR δ (400 MHz, CDCl₃): 0.85 (t, J=6.8 Hz, 3H), 1.18-1.30 (m, 32H), 1.48-1.57 (m, 2H), 2.42-2.48 (m, 2H), 5.75 (d, J=2.6 Hz, 1H), 11.21 (s, 1H), 12.19 (s, 1H).

### Example 127

### 3-Chloro-5-(2,2-dimethyldodecyl)-1H-pyrrole-2-carboxylic acid

To a solution of methyl 3-chloro-5-(2,2-dimethyldodecyl)-1H-pyrrole-2-carboxylate (Intermediate 59b, 115 mg, 0.32 mmol) in ethanol (1 mL) and water (0.5 mL) was added sodium hydroxide (39 mg, 0.97 mmol) and the resulting mixture was heated at 80° C. for 1 h. The organic solvent was evaporated, water was added and pH was adjusted to pH=2 by addition of 1M hydrochloric acid solution. The white solid formed was filtered, washed with water and dried to give the title compound (82 mg, 74%).

MS (m/z) 342 [M+1]⁺.

¹H NMR δ (400 MHz, DMSO-d6): 0.78 (s, 6H), 0.83-1.02 (m, 3H), 1.07-1.16 (m, 2H), 1.16-1.35 (m, 16H), 2.36 (s, 2H), 5.69 (s, 1H).

### Example 128

### 3-Chloro-5-(3,3-difluorododecyl)-1H-pyrrole-2-carboxylic acid

Obtained as a white solid (59%) from methyl 3-chloro-5-(3,3-difluorododecyl)-1H-pyrrole-2-carboxylate (Intermediate 60c) following the experimental procedure described in Example 64.

¹H-NMR δ (400 MHz, CDCl₃): 0.88 (t, J=7 Hz, 3H), 1.20-1.36 (m, 12H), 1.44-1.49 (m, 2H), 1.77-1.90 (m, 2H), 2.07-2.20 (m, 2H), 2.79-2.83 (m, 2H), 6.06 (d, J=3 Hz, 1H), 9.04 (brs, 1H).

### Example 129

### 3-Cyano-5-dodecyl-1H-pyrrole-2-carboxylic acid

Obtained as a white solid (71%) from ethyl 3-cyano-5-dodecyl-1H-pyrrole-2-carboxylate (Intermediate 61) following the experimental procedure described in Example 12.

MS (m/z): 305 [M+1]⁺.

¹H NMR δ (400 MHz, DMSO-d6): 0.81-0.88 (m, 3H), 1.20-1.26 (m, 18H), 1.48-1.59 (m, 2H), 2.51-2.56 (m, 2H), 6.36 (s, 1H).

### Example 130

### 3-Chloro-5-dodecyl-1-methyl-1H-pyrrole-2-carboxylic acid

Obtained as a white solid (75%) from methyl 3-chloro-5-dodecyl-1-methyl-1H-pyrrole-2-carboxylate (Intermediate 62) following the experimental procedure described in Example 56.

MS (m/z): 328 [M+1]⁺.

¹H NMR δ (400 MHz, CDCl₃): 0.86-0.90, (m, 3H), 1.21-1.43 (m, 18H), 1.52-1.66 (m, 2H), 2.44-2.57 (m, 2H), 3.77 (s, 3H), 5.96 (s, 1H).

### Example 131

### 3-Fluoro-5-(14-fluorotetradecyl)-1H-pyrrole-2-carboxylic acid

Obtained as a white solid (38%) from ethyl 3-fluoro-5-(14-fluorotetradecyl)-1H-pyrrole-2-carboxylate (Intermediate 63b) following the experimental procedure described in Example 56 followed by purification by preparative HPLC-MS (gradient from water to ACN/methanol 1:1).

MS (m/z): 344 [M+1]⁺.

¹H NMR δ (400 MHz, Methanol-d4): 1.25-1.44 (m, 20H), 1.53-1.74 (m, 4H), 2.49 (t, J=7.6 Hz, 2H), 4.34 (t, J=6.1 Hz, 1H), 4.46 (t, J=6.1 Hz, 1H), 5.58 (s, 1H).

### Example 132

### 3-Fluoro-4-hexadecyl-1H-pyrrole-2-carboxylic acid

Obtained as a white solid (76%) from ethyl 3-fluoro-4-hexadecyl-1H-pyrrole-2-carboxylate (Intermediate 64b) following the experimental procedure described in Example 21.

MS (m/z): 352 [M−1]⁺.

¹H-NMR δ (400 MHz, DMSO-d6): 0.85 (t, J=6.8 Hz, 3H), 1.17-1.30 (m, 24H), 1.41-1.48 (m, 2H), 2.32 (t, J=7 Hz, 2H), 6.32 (s, 1H), 10.38-10.71 (bs, 1H).

Pharmacological Activity

In Vitro Assay of Inhibition of Lipid Synthesis

To evaluate the inhibition of lipid synthesis, the immortalized human sebocyte cell line, SZ95 (stablished by Zouboulis, C. C. et al J Invest Dermatol 1999; 113:1011-20), was treated with arachidonic acid (AA) in presence or absence of compound. Lipids were detected by using a lipid sensing fluorophore.

Compounds were dissolved in dimethylsulfoxide (DMSO) 100%. Then the stocks were serial diluted 1/3 in DMSO 100%, and this battery of solutions were diluted 1/10 in culture medium, to minimize the percentage of the DMSO over cells.

10 k cells were plated in 384 well microtiter plates and incubated at 37° C. and 5% CO2 in DMEM/F12 supplemented with 10% FBS, 1.25 ng/ml of rhEGF and GA-1000 before of compound and stimulus addition. After 24 h, compounds dissolved in culture media were added over cells, diluting the solutions prepared 1/40 in the final volume of the assay. Then, cells and compounds were preincubated for 30 min at 37° C. and 5% CO2. After this prior incubation, the lipid synthesis was induced by 75 μM of AA final solution, preparing a solution 10× in culture media. Finally, SZ95 treated were incubated for 48 h at 37° C. and 5% CO2.

Neutral lipids were measured using AdipoRed™, purchased from LONZA. To do that, cells were washed with PBS and incubated with a solution of AdipoRed™ (final dilution 1/80 in PBS) for 30 min at room temperature. After the staining process, the fluorescence intensity (FI) was quantified using a fluorescence plate reader (excitation 485 nm; emission 535).

Activity of compounds were calculated as % of inhibition considering the maximal fluorescence for AA-stimulated cells and the minimum fluorescence for unstimulated cells as controls.

Some of the acronyms used above have the following meaning:
AA: Arachidoin Acid
DMSO: dimethylsulfoxide
DMEM/F12: Dulbecco's Modified Eagle's Medium/F12
FBS: Fetal Bovine Serum
rhEGF: recombinant human Epidermal Growth Factor
GA: Gentamicin/Amphotericin
PBS: Phosphate-buffered saline
FI: Fluorescece intensity

In the following table 1, IC₅₀ values are represented by letters according to the value:

TABLE 1

| | Inhibition of Lipid |
|---|---|
| | Synthesis assay IC₅₀ |
| Example | (nM) |
| 1 | B |
| 2 | D |
| 3 | C |
| 4 | C |
| 5 | B |
| 6 | C |
| 7 | D |
| 8 | A |
| 9 | C |
| 10 | C |
| 11 | A |
| 12 | A |
| 13 | A |
| 14 | A |
| 15 | A |
| 16 | A |
| 17 | A |
| 18 | A |
| 19 | A |
| 20 | C |
| 21 | A |
| 22 | C |
| 23 | D |
| 24 | B |
| 25 | C |
| 26 | A |
| 27 | A |
| 28 | A |
| 29 | A |
| 30 | A |
| 31 | A |
| 32 | B |
| 33 | A |
| 34 | A |
| 35 | A |
| 36 | A |
| 37 | A |
| 38 | B |
| 39 | A |
| 40 | A |
| 41 | A |
| 42 | A |
| 43 | A |
| 44 | A |
| 45 | A |
| 46 | D |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | B |
| 60 | C |
| 61 | A |
| 62 | B |
| 63 | B |
| 64 | A |
| 65 | A |
| 66 | A |
| 67 | B |
| 68 | B |
| 69 | C |
| 70 | C |
| 71 | A |
| 72 | A |
| 73 | A |
| 74 | A |
| 75 | A |
| 76 | C |
| 77 | A |
| 78 | A |
| 79 | A |
| 80 | A |
| 81 | A |
| 82 | A |
| 83 | B |
| 84 | A |
| 85 | A |
| 86 | A |
| 87 | A |
| 88 | A |
| 89 | A |
| 90 | A |
| 91 | C |
| 92 | A |
| 93 | A |
| 94 | A |
| 95 | A |
| 96 | A |
| 97 | A |
| 98 | B |
| 99 | A |
| 100 | B |
| 101 | C |
| 102 | A |
| 103 | A |
| 104 | A |
| 105 | A |
| 106 | A |
| 107 | A |
| 108 | B |
| 109 | B |
| 110 | A |
| 111 | A |
| 112 | A |
| 113 | C |
| 114 | C |
| 115 | A |
| 116 | A |
| 117 | A |
| 118 | B |
| 119 | A |
| 120 | A |
| 121 | A |
| 122 | A |
| 123 | A |
| 124 | A |
| 125 | A |
| 126 | A |
| 127 | B |
| 128 | B |
| 129 | C |
| 130 | D |
| 131 | A |
| 132 | A |

A: <250 nM
B: 250-<1000 nM
C: 1000-5000 nM
D: >5000 nM

It can be seen from Table 1 that the pyrrole derivatives of the present invention are potent inhibitors of lipid synthesis. Preferred pyrrole derivatives of the invention possess an IC₅₀ value for the inhibition of lipid synthesis (determined as defined above) of less than 1 μM (1000 nM), preferably of less than 0.25 μM (250 nM). More preferred pyrrole derivatives of the invention possess an IC₅₀ value for the inhibition of lipid synthesis of less than 100 nM, preferably of less than 50 nM and more preferably of less that 10 nM.

In the following table 2, IC₅₀ values for pyrrole derivatives of the invention that posses an IC₅₀ value of less than 250 nM, are represented by letter codes according to the value:

TABLE 2

| | Inhibition of Lipid |
|---|---|
| | Synthesis assay IC50 |
| Example | (nM) |
| 8 | A+ |
| 11 | A+ |
| 12 | A+++ |
| 13 | A+++ |
| 14 | A+++ |
| 15 | A+++ |
| 16 | A++ |
| 17 | A+++ |
| 18 | A+ |
| 19 | A+++ |
| 21 | A+++ |
| 26 | A+ |
| 27 | A+++ |
| 28 | A++ |
| 29 | A+ |
| 30 | A+++ |
| 31 | A+++ |
| 33 | A++ |
| 34 | A+ |
| 35 | A+ |
| 36 | A+ |
| 37 | A+ |
| 39 | A+++ |
| 40 | A+++ |
| 41 | A+++ |
| 42 | A+ |
| 43 | A+++ |
| 44 | A+++ |
| 45 | A++ |
| 47 | A++ |
| 48 | A+++ |
| 49 | A+++ |
| 50 | A+++ |
| 51 | A+++ |
| 52 | A+++ |
| 53 | A+++ |
| 54 | A+++ |
| 55 | A++ |
| 56 | A++ |
| 57 | A+++ |
| 58 | A+++ |
| 61 | A+ |
| 64 | A++ |
| 65 | A++ |
| 66 | A+ |
| 71 | A+++ |
| 72 | A+++ |
| 73 | A+++ |
| 74 | A+++ |
| 75 | A+++ |
| 77 | A++ |
| 78 | A+++ |
| 79 | A+++ |
| 80 | A+++ |
| 81 | A+ |
| 82 | A+ |
| 84 | A+ |
| 85 | A+++ |
| 86 | A+++ |
| 87 | A+++ |
| 88 | A+++ |
| 89 | A+++ |
| 90 | A++ |
| 92 | A+++ |
| 93 | A+++ |
| 94 | A+ |
| 95 | A+++ |
| 96 | A++ |
| 97 | A+ |
| 99 | A+ |
| 102 | A+++ |
| 103 | A+++ |
| 104 | A+ |
| 105 | A+++ |
| 106 | A+++ |
| 107 | A+++ |
| 110 | A+ |
| 111 | A+ |
| 112 | A+ |
| 115 | A+ |
| 116 | A++ |
| 117 | A+ |
| 119 | A++ |
| 120 | A+++ |
| 121 | A+++ |
| 122 | A++ |
| 123 | A++ |
| 127 | A+++ |
| 128 | A+++ |
| 130 | A+ |
| 131 | A+ |
| 132 | A+++ |

A+++: <50 nM
A++: 50-<100 nM
A+: 100-<250 nM

The invention is also directed to a compound of the invention as described herein for use in the treatment of the human or animal body by therapy. Compounds of the invention intended for pharmaceutical use may be administered as crystalline or amorphous products, or mixtures thereof. They may be obtained, for example, as solid plugs, powders, or films by methods such as precipitation, crystallization, freeze drying, spray drying, or evaporative drying. Microwave or radio frequency drying may be used for this purpose.

Combinations

The pyrrole derivatives of the present invention may also be combined with other active compounds in the treatment of a pathological condition or disease susceptible to amelioration by inhibition of Acetyl-CoA carboxylase (ACC).

The combinations of the invention can optionally comprise one or more additional active substances which are known to be useful in the treatment of a dermatological disease, an inflammatory or autoimmune-mediated disease and a metabolism/endocrine function disorder; more in particular wherein the pathological condition or disease is selected from acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis, such as,
a) Corticoids and glucocorticoids, such as beclomethasone, betamethasone, betamethasone dipropionate, budesonide, dexamethasone, fluticasone furoate, fluticasone propionate, hydrocortisone, methylprednisolone, mometasone furoate, prednicarbate, prednisolone or prednisone;
b) Dihydrofolate reductase inhibitors, such as methotrexate or pralatrexate;
c) Dihydroorotate dehydrogenase (DHODH) inhibitors such as leflunomide, teriflunomide or
ASLAN-003 or LAS 186323;
d) Purine antagonists, such as azathioprine, mercaptopurine or tioguanine;
e) Antimalarials, such as hydroxichloroquine, chloroquine or quinacrine;
f) Calcineurin inhibitors, such as cyclosporine A, tacrolimus, pimecrolimus or voclosporin;
g) Inosine-monophosphate dehydrogenase (IMPDH) inhibitors, such as mycophenolate mophetyl, ribavirin or mizoribine;
h) Fumaric acid esters, such as dimethyl fumarate;
i) Vitamine D3 derivatives such as calcipotriol, calcitriol or tacalcitol;
j) Retinoids, such as tazarotene, adapalene, tretinoin alitretinoin, acitretin or isotretinoin;
k) Anti-tumor necrosis factor-alpha (Anti-TNF-alpha) monoclonal antibodies, such as infliximab, adalimumab, certolizumab pegol orgolimumab;
l) Soluble Tumor necrosis factor-alpha (TNF-alpha) receptors such as etanercept or CC-11050;
m) Anti-Interleukin 6 Receptor (IL-6R) antibody, such as tocilizumab, sarilumab, SA-237 or ALX-0061;
n) Anti-Interleukin 12 (IL-12)/Interleukin 23 (IL-23) antibody, such as ustekinumab;
o) Anti-Interleukin 17 Receptor (IL-17R) antibody, such as brodalumab;
p) Anti-CD20 (B lymphocyte protein) antibody, such as rituximab, ofatumumab, obinutuzumab, ocrelizumab, ublituximab, veltuzumab, or ocaratuzumab;
q) Anti-Interleukin 5 (IL-5) antibody, such as mepolizumab;
r) Anti-Interleukin 5 Receptor (IL-5R) antibody, such as benralizumab;
s) Anti-Interleukin 13 (IL-13) antibody, such as lebrikizumab or tralokinumab;
t) Anti-Interleukin 4 Receptor (IL-4R)/Interleukin 13 Receptor (IL-13R) antibody, such as dupilumab;
u) Anti-Interleukin 17 (IL-17) antibody, such as secukinumab, ixekizumab or bimekizumab;
v) An anti-IL-23 antibody such as tildrakizumab, guselkumab or risankizumab;
w) Anti-Interleukin 1 Receptor (IL-1R) antibody;
x) Anti-Immunoglobuline E (IgE) antibody, such as omalizumab or quilizumab;
y) Anti-B-cell activating factor (BAFF), such as belimumab or atacicept;
z) Anti-CD19 (B lymphocyte protein) monoclonal antibody, such as blinatumomab, MEDI-551 or MOR-208;
aa) Kappa opioid agonists, such as nalfurafine, nalbuphine, asimadoline or CR-845;
bb) Neurokinin receptor 1 antagonists, such as aprepitant, fosaprepitant, rolapitant, orvepitant, tradipitant or serlopitant;
cc) Dihydropteroate synthase inhibitors, such as dapsone or sulfadoxine;
dd) Histamine 1 (H1) receptor antagonists, such as azelastine, ebastine, desloratadine, promethazine, mizolastine or cetirizine;
ee) Cysteinyl leukotriene (CysLT) receptor antagonists, such as montelukast, zafirlukast, tipelukast or masilukast;
ff) Chemoattractant receptor homologous molecule expressed on TH2 cells (CRTh2) antagonists, such as OC-459, AZD-1981, ADC-3680, ARRY-502 or setipripant;
gg) Topical anti-septics, such as Benzoyl peroxide (BPO), triclosan, chlorhexidine, crystal violet 0.3% or sodium hypochlorite water-baths;
hh) Antibiotics such as tetracyclines (doxycycline, minocycline, and tetracycline) macrolides (azithromycin, clarithromycin, erythromycin) or clindamycin;
ii) Azelaic acid;
jj) α-hydroxy acids such as glycolic acid or lactic acid;
kk) β-hydroxy acids such as salycilic acid; and
ll) A PDE4 inhibitor such as apremilast.

The pyrrole derivatives of the present invention and the combinations of the invention may be used in the treatment of a dermatological disease, an inflammatory or autoimmune-mediated disease and a metabolism/endocrine function disorder; more in particular wherein the pathological condition or disease is selected from acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis; preferably in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis.

In a preferred embodiment the pyrrole derivatives of the present invention and the combinations of the invention may be used in the treatment of dermatological diseases.

In a more preferred embodiment, the pyrrole derivatives of the present invention and the combinations of the invention may be used in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis.

The active compounds in the combination product may be administered together in the same pharmaceutical composition or in different compositions intended for separate, simultaneous, concomitant or sequential administration by the same or a different route.

It is contemplated that all active agents would be administered at the same time, or very close in time. Alternatively, one or two actives could be administered in the morning and the other(s) later in the day. Or in another scenario, one or two actives could be administered twice daily and the other(s) once daily, either at the same time as one of the twice-a-day dosing occurred, or separately. Preferably at least two, and more preferably all, of the actives would be administered together at the same time. Preferably, at least two, and more preferably all actives would be administered as an admixture.

The invention is also directed to a combination product of the pyrrole derivatives of the invention together with one or more other therapeutic agents for use in the treatment of a pathological condition or disease susceptible to amelioration by inhibition of Acetyl-CoA carboxylase (ACC), in particular wherein the pathological condition or disease is selected from a dermatological disease, an inflammatory or autoimmune-mediated disease and a metabolism/endocrine function disorder. More in particular wherein the pathological condition or disease is selected from acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis; preferably in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis.

The invention also encompasses the use of a combination of the pyrrole derivatives of the invention together with one or more other therapeutic agents for the manufacture of a formulation or medicament for treating these diseases.

The invention also provides a method of treatment of a pathological condition or disease susceptible to amelioration by inhibition of Acetyl-CoA carboxylase (ACC), in particular wherein the pathological condition or disease is selected from a dermatological disease, an inflammatory or autoimmune-mediated disease and a metabolism/endocrine function disorder. More in particular wherein the pathological condition or disease is selected from acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis; preferably in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis, comprising administering a therapeutically effective amount of a combination of the pyrrole derivatives of the invention together with one or more other therapeutic agents.

The active compounds in the combinations of the invention may be administered by any suitable route, depending on the nature of the disorder to be treated, e.g. orally (as syrups, tablets, capsules, lozenges, controlled-release preparations, fast-dissolving preparations, etc); topically (as creams, ointments, lotions, nasal sprays or aerosols, etc) or by injection (subcutaneous, intradermic, intramuscular, intravenous, etc).

The active compounds in the combination, i.e. the pyrrole derivatives of the invention, and the other optional active compounds may be administered together in the same pharmaceutical composition or in different compositions intended for separate, simultaneous, concomitant or sequential administration by the same or a different route.

One execution of the present invention consists of a kit of parts comprising a pyrrole derivative of the invention together with instructions for simultaneous, concurrent, separate or sequential use in combination with another active compound useful in the treatment of acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis; preferably in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis.

Another execution of the present invention consists of a package comprising a pyrrole derivative of the invention and another active compound useful in the treatment of acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis; preferably in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis.

Pharmaceutical Compositions

Pharmaceutical compositions according to the present invention comprise the pyrrole derivatives of the invention in association with a pharmaceutically acceptable diluent or carrier.

As used herein, the term pharmaceutical composition refers to a mixture of one or more of the pyrrole derivatives of the invention or prodrugs thereof, with other chemical components, such as physiologically/pharmaceutically acceptable carriers and excipients. The purpose of a pharmaceutical composition is to facilitate administration of a compound to an organism.

As used herein, a physiologically/pharmaceutically acceptable diluent or carrier refers to a carrier or diluent that does not cause significant irritation to an organism and does not abrogate the biological activity and properties of the administered compound.

The invention further provides pharmaceutical compositions comprising the pyrrole derivatives of the invention in association with a pharmaceutically acceptable diluent or carrier together with one or more other therapeutic agents for use in the treatment of a pathological condition or disease susceptible to amelioration by inhibition of Acetyl-CoA carboxylase (ACC), such as the ones previously described.

The invention is also directed to pharmaceutical compositions of the invention for use in the treatment of a pathological condition or disease susceptible to amelioration by inhibition of Acetyl-CoA carboxylase (ACC), in particular wherein the pathological condition or disease is selected from a dermatological disease, an inflammatory or autoimmune-mediated disease and a metabolism/endocrine function disorder. More in particular wherein the pathological condition or disease is selected from acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis; preferably in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis.

The invention also encompasses the use of a pharmaceutical composition of the invention for the manufacture of a medicament for treating these diseases.

The invention also provides a method of treatment of a pathological condition or disease susceptible to amelioration by inhibition of Acetyl-CoA carboxylase (ACC), in particular wherein the pathological condition or disease is selected from a dermatological disease, an inflammatory or autoimmune-mediated disease and a metabolism/endocrine function disorder. More in particular wherein the pathological condition or disease is selected from acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis and palmoplantar pustulosis; preferably in the treatment of acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis, comprising administering a therapeutically effective amount of a pharmaceutical composition of the invention.

The present invention also provides pharmaceutical compositions which comprise, as an active ingredient, at least a pyrrole derivative of the invention in association with a pharmaceutically acceptable excipient such as a carrier or diluent. Preferably the compositions are made up in a form suitable for oral, topical, nasal, rectal, percutaneous or injectable administration. The compounds of the present invention show physicochemical properties (such as solubility water and in a range of lipophilic and hydrophilic solvents, melting point and stability), which make them specially suitable for topical administration.

In a preferred embodiment, the compositions are made up in a form suitable for topical administration.

Pharmaceutical compositions suitable for the delivery of pyrrole derivatives of the invention and methods for their preparation will be readily apparent to those skilled in the art. Such compositions and methods for their preparation can be found, for example, in Remington: The Science and Practice of Pharmacy, 21st Edition, Lippincott Williams & Wilkins, Philadelphia, Pa., 2001.

i) Topical Administration

The pyrrole derivatives of the invention may be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Other means of topical administration include delivery by electroporation, iontophoresis, phonophoresis, sonophoresis and microneedle or needle-free injection.

Formulations for topical administration may be formulated to be immediate and/or modified release. Modified release formulations include delayed-, sustained-, pulsed-, controlled-, targeted and programmed release.

ii) Oral Administration

The pyrrole derivatives of the invention may be administered orally (peroral administration; per os (latin)). Oral administration involve swallowing, so that the compound is absorbed from the gut and delivered to the liver via the portal circulation (hepatic first pass metabolism) and finally enters the gastrointestinal (GI) tract.

Compositions for oral administration may take the form of tablets, retard tablets, sublingual tablets, capsules, inhalation aerosols, inhalation solutions, dry powder inhalation, or liquid preparations, such as mixtures, solutions, elixirs, syrups or suspensions, all containing the compound of the invention; such preparations may be made by methods well-known in the art. The active ingredient may also be presented as a bolus, electuary or paste.

iii) Oral Mucosal Administration

The pyrrole derivatives of the invention can also be administered via the oral mucosal. Within the oral mucosal cavity, delivery of drugs is classified into three categories: (a) sublingual delivery, which is systemic delivery of drugs through the mucosal membranes lining the floor of the mouth, (b) buccal delivery, which is drug administration through the mucosal membranes lining the cheeks (buccal mucosa), and (c) local delivery, which is drug delivery into the oral cavity.

Pharmaceutical products to be administered via the oral mucosal can be designed using mucoadhesive, quick dissolve tablets and solid lozenge formulations, which are formulated with one or more mucoadhesive (bioadhesive) polymers and/or oral mucosal permeation enhancers.

iv) Inhaled Administration

The pyrrole derivatives of the invention can also be administered by inhalation, typically in the form of a dry powder from a dry powder inhaler or as an aerosol spray from a pressurized container, pump, spray, atomizer (preferably an atomizer using electrohydrodynamics to produce a fine mist), or nebulizer, with or without the use of a suitable propellant.

v) Nasal Mucosal Administration

The pyrrole derivatives of the invention may also be administered via the nasal mucosal.

Typical compositions for nasal mucosa administration are typically applied by a metering, atomizing spray pump and are in the form of a solution or suspension in an inert vehicle such as water optionally in combination with conventional excipients such as buffers, anti-microbials, tonicity modifying agents and viscosity modifying agents

vi) Parenteral Administration

The pyrrole derivatives of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art. The solubility of compounds of the invention used in the preparation of parenteral solutions may be increased by the use of appropriate formulation techniques, such as the incorporation of solubility-enhancing agents.

vii) Rectal/Intravaginal Administration

The pyrrole derivatives of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

viii) Ocular Administration

The pyrrole derivatives of the invention may also be administered directly to the eye or ear, typically in the form of drops of a micronized suspension or solution in isotonic, pH-adjusted, sterile saline. Other formulations suitable for ocular and aural administration include ointments, biodegradable {e.g. absorbable gel sponges, collagen) and nonbiodegradable (e.g. silicone) implants, wafers, lenses and particulate or vesicular systems, such as niosomes or liposomes. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The amount of the active pyrrole derivative of the invention administered will be dependent on the subject being treated, the severity of the disorder or condition, the rate of administration, the disposition of the compound and the discretion of the prescribing physician. However, an effective dosage is typically in the range of 0.01-3000 mg, more preferably 0.5-1000 mg of active ingredient or the equivalent amount of a pharmaceutically acceptable salt thereof per day. Daily dosage may be administered in one or more treatments, preferably from 1 to 4 treatments, per day.

Preferably, the pharmaceutical compositions of the invention are made up in a form suitable for oral or topical administration, being particularly preferred topical administration.

The amount of each active which is required to achieve a therapeutic effect will, of course, vary with the particular active, the route of administration, the subject under treatment, and the particular disorder or disease being treated.

1. A compound of Formula (I), or a pharmaceutically acceptable salt, or a solvate, or a N-oxide, or a tautomer, or a stereoisomer, or an isotopically-labelled derivative thereof: 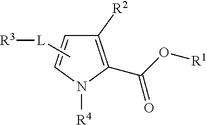
Formula (I)
wherein:
R¹ is chosen from a hydrogen atom, a linear or branched C₁₋₄ alkyl group, a linear or branched C₁₋₄haloalkyl group, a linear or branched C₁₋₁₀ hydroxyalkyl group, a —(CH₂)₀₋₃—(C₃₋₇ monocyclic cycloalkyl group), a —(CH₂)₀₋₃-(monocyclic or bicyclic C₆₋₁₄ aryl group), a —(CH₂)₀₋₃-(4- to 7-membered heterocyclyl group containing at least one heteroatom chosen from N, O, and S), a —(CH₂)₀₋₃-(monocyclic or bicyclic 5- to 14-membered heteroaryl group containing at least one heteroatom chosen from N, O, and S), a —(CH₂)₀₋₄—[(CH₂)₁₋₃—O]₁₋₅—R^{a} group, a —(CR^{a}R^{b})₁₋₃—OC(O)—R⁵ group, and a —(CH₂)₁₋₃—C(O)NR⁵R^{a} group, wherein the cycloalkyl, aryl, heterocyclyl and heteroaryl groups are unsubstituted or substituted by one or more substituents chosen from a halogen atom, a linear or branched C₁₋₄ alkyl group and an oxo group;
R² is chosen from a hydrogen atom, halogen atom, a —CN group and a linear or branched C₁₋₄ alkyl group;
R³ is a linear or branched C₉₋₂₀ alkyl group,
wherein the alkyl group is unsubstituted or substituted by one or more substituents chosen from a halogen atom, a hydroxyl group, a linear or branched C₁₋₄ alkyl group, a linear or branched C₁₋₆alkoxy group, and a linear or branched C₁₋₄ hydroxyalkyl group;
R⁴ is chosen from a hydrogen atom and a linear or branched C₁₋₄ alkyl group;
R⁵ is chosen from a hydrogen atom, linear or branched C₁₋₁₀ alkyl group, a —O-(linear or branched C₁₋₁₀ alkyl group), a —O—(CH₂)₀₋₃—(C₃₋₇ monocyclic cycloalkyl group), a —O—(CH₂)₀₋₃-(monocyclic or bicyclic C₆₋₁₄ aryl group), a —(CH₂)₀₋₃C(O)OR^{a} group, and a —O—[(CH₂)₁₋₃—O]₁₋₅—R^{a} group;
wherein the alkyl group is unsubstituted or substituted by one or more substituents chosen from a halogen atom, hydroxyl group, and an amino group;
R^{a} and R^{b} are independently chosen from a hydrogen atom and a linear or branched C₁₋₄ alkyl group; wherein the alkyl group is unsubstituted or substituted by one or more substituents chosen from a halogen atom and a hydroxyl group; and
L is a direct bond, a —(CH₂)₀₋₄—O— group, a —(CH₂)₀₋₄—S— group, a —(CH₂)₀₋₄—NRₐ— group, a —C(O)NR^{a}— group, a —NR^{a}C(O)— group or a carbonyl group; characterised in that when R² is a hydrogen atom, L is a —(CH₂)₀₋₄—O— group, or a —C(O)NR^{a}— group.

2. The compound according to claim 1, wherein the compound of Formula (I) is Formula (Ia):

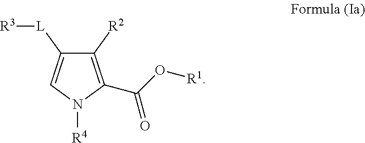

3. The compound according to claim 1, wherein the compound of Formula (I) is Formula (Ib):

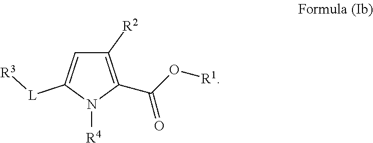

4. The compound according to claim 1, wherein R² represents is a halogen atom.

5. The compound according to claim 4, wherein R² is a fluorine or chlorine atom.

6. The compound according to claim 1, wherein R³ represents is a linear or branched C₉₋₂₀ alkyl group,
wherein the alkyl group is unsubstituted or substituted by one or more substituents chosen from a halogen atom, a hydroxyl group, a linear or branched C₁₋₄ alkyl group, and a linear or branched C₁₋₃alkoxy group.

7. The compound according to claim 1, wherein L is a direct bond or —O—.

8. The compound according to claim 1, wherein:
R² is a halogen atom;
R³ is a linear or branched C₉₋₂₀ alkyl group,
wherein the alkyl group is unsubstituted or substituted by one or more substituents chosen from a halogen atom, a hydroxyl group, a linear or branched C₁₋₄ alkyl group, and a linear or branched C₁₋₃alkoxy group; and
L is a direct bond or —O—.

9. The compound according to claim 1, wherein the compound of Formula (I) is Formula (Ia): 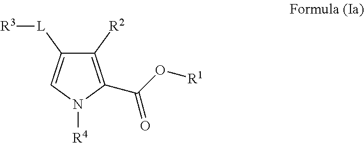
wherein:
R¹ is chosen from a hydrogen atom, a linear or branched C₁₋₄ alkyl group, a linear or branched C₁₋₄haloalkyl group, a linear or branched C₂₋₁₀ hydroxyalkyl group, a cyclohexyl group, a —CH₂-phenyl group, a —(CH₂)₁₋₂-(5- to 6-membered heterocyclyl group containing at least one heteroatom chosen from N, O_{A} and S), a —(CH₂CH₂O)₁₋₄—R^{a} group, a —(CR^{a}R^{b})₁₋₃—OC(O)—R⁵ group and a —(CH₂)₁₋₃—C(O)NR⁵R^{a} group,
wherein the cyclohexyl, phenyl and heterocyclyl groups are unsubstituted or substituted by one or more substituents chosen from a halogen atom, a linear or branched C₁₋₄ alkyl group, and an oxo group;
R² is a halogen atom;
R³ is a linear or branched C₁₀₋₁₇ alkyl group, wherein the alkyl group is unsubstituted or substituted by one or more substituents chosen from a halogen atom, a hydroxyl group, a linear or branched C₁₋₄ alkyl group, and a linear or branched C₁₋₃alkoxy group;
R⁴ is a hydrogen atom;
R⁵ is chosen from a —O-(linear or branched C₁₋₁₀alkyl group), a —O-cyclohexyl group, a —O—CH₂-phenyl group, a —(CH₂)₁₋₂C(O)OR^{a} group, a —O—(CH₂CH₂O)₁₋₃—R^{a} group, and a —O—CH₂CH₂CH₂O—R³ group;
R^{a} is chosen from a hydrogen atom and a linear or branched C₁₋₄ alkyl group, wherein the alkyl group is unsubstituted or substituted by one or more substituents chosen from a halogen atom and hydroxyl group;
R^{b} is a hydrogen atom; and
L is a direct bond or —O—.

10. The compound according to claim 9, wherein:
R¹ is chosen from a hydrogen atom, a linear or branched C₁₋₃haloalkyl group, a linear or branched C₃₋₉ hydroxyalkyl group, a —(CH₂)₁₋₂-(5-membered heterocyclyl group containing at least one heteroatom chosen from N and O), a —(CH₂CH₂O)₂—R^{a} group, a —(CR^{a}R^{b})—OC(O)—R⁵ group and a —(CH₂)—C(O)NR⁵R^{a} group,
wherein the heterocyclyl group is unsubstituted or substituted by one or more substituents chosen from a linear or branched C₁₋₄ alkyl group and an oxo group;
R² is a fluorine atom or a chlorine atom;
R³ is a linear or branched C₁₀₋₁₇ alkyl group, wherein the alkyl group is unsubstituted or substituted by one or more substituents chosen from a fluorine atom, a linear or branched C₁₋₄ alkyl group, and a linear or branched C₁₋₃ alkoxy group;
R⁵ is chosen from the group consisting of a —O-(linear or branched C₂₋₄ alkyl group), a —O-cyclohexyl group, a —O—CH₂-phenyl group, a —(CH₂)—C(O)OR^{a} group, a —O—(CH₂CH₂O)₁₋₂—R^{a} group, and a —O—CH₂CH₂CH₂O—R³ group;
R^{a} is chosen from a hydrogen atom and a linear or branched C₁₋₄ alkyl group; wherein the alkyl group is unsubstituted or substituted by one or more substituents chosen from a halogen atom and hydroxyl group.

11. The compound according to claim 1, wherein:
R¹ is chosen from a hydrogen atom, a linear or branched C₁₋₄ alkyl group, a —CH₂CF₃ group, a —(CH₂)₂₋₉—OH group, a —CH₂—CH(OH)—CH₂—OH, a —CH(CH₂OH)₂ group, a cyclohexyl group, a —(CH₂)₂-(2,5-dioxopyrrolidin-1-yl) group, a —(CH₂)₂-(2-oxopyrrolidin-1-yl) group, a —(CH₂)-(5-methyl-2-oxo-1,3-dioxol-4-yl) group, a —CH₂-phenyl group, a —(CH₂CH₂O)₂₋₄—R^{a} group, a —CH(CH₃)—OC(O)OCH(CH₃)₂ group, a —CH(CH₃)—OC(O)OC(CH₃)₃ group, a —CH(CH₃)—OC(O)O(CH₂)₈CH₃ group, a —CH(CH₃)—OC(O)O-cyclohexyl group, a —CH(CH₃)—OC(O)O—CH₂-phenyl group, a —CH(CH₃)—OC(O)O(CH₂CH₂O)₁₋₂—R^{a} group, a —CH(CH₃)—OC(O)O(CH₂)₃OH group, a —(CH₂)₂—OC(O)C(NH₂)—CH(CH₃)₂ group, and a —CH₂—C(O)N(CH₃)CH₂CO₂R^{a} group;
R² is a hydrogen atom, methyl group, fluorine atom, chlorine atom, bromine atom, or a —CN group;
R³ is a linear C₉₋₁₈ alkyl group,
wherein the alkyl group is unsubstituted or substituted by one or more substituents selected from a fluorine atom, a linear or branched C₁₋₄ alkyl group, and a linear or branched C₁₋₃alkoxy group;
R⁴ is chosen from the group consisting of a hydrogen atom and a linear or branched C₁₋₄ alkyl group;
R^{a} is chosen from the group consisting of a hydrogen atom and a linear or branched C₁₋₄ alkyl group;
L is a direct bond, —O—, —S—, or a carbonyl group; characterised in that when R² is a hydrogen atom, L is a —O—.

12. The compound according to claim 1, wherein the compound is chosen from:
4-(Dodecyloxy)-1H-pyrrole-2-carboxylic acid;
Ethyl 4-(dodecyloxy)-1H-pyrrole-2-carboxylate;
2-(2,5-Dioxopyrrolidin-1-yl)ethyl 4-(dodecyloxy)-1H-pyrrole-2-carboxylate;
2-(2-Oxopyrrolidin-1-yl)ethyl 4-(dodecyloxy)-1H-pyrrole-2-carboxylate;
2,2,2-trifluoroethyl 4-(dodecyloxy)-1H-pyrrole-2-carboxylate;
2-Hydroxyethyl 4-(dodecyloxy)-1H-pyrrole-2-carboxylate;
2-(2-(2-(2-hydroxyethoxy)ethoxy)ethoxy)ethyl 4-(dodecyloxy)-1H-pyrrole-2-carboxylate;
1-((isopropoxycarbonyl)oxy)ethyl 4-(dodecyloxy)-1H-pyrrole-2-carboxylate;
2-((2-ethoxy-2-oxoethyl)(methyl)amino)-2-oxoethyl 4-(dodecyloxy)-1H-pyrrole-2-carboxylate;
2-((L-valyl)oxy)ethyl 4-(dodecyloxy)-1H-pyrrole-2-carboxylate;
(5-methyl-2-oxo-1,3-dioxol-4-yl)methyl 4-(dodecyloxy)-1H-pyrrole-2-carboxylate;
4-Decyl-3-fluoro-1H-pyrrole-2-carboxylic acid;
3-Fluoro-4-undecyl-1H-pyrrole-2-carboxylic acid;
4-Dodecyl-3-fluoro-1H-pyrrole-2-carboxylic acid;
2,2,2-trifluoroethyl 4-dodecyl-3-fluoro-1H-pyrrole-2-carboxylate;
2-(2-ethoxyethoxy)ethyl 4-dodecyl-3-fluoro-1H-pyrrole-2-carboxylate;
1-((Isopropoxycarbonyl)oxy)ethyl 4-dodecyl-3-fluoro-1H-pyrrole-2-carboxylate;
1-(((2-Methoxyethoxy)carbonyl)oxy)ethyl 4-dodecyl-3-fluoro-1H-pyrrole-2-carboxylate;
4-oxo-3,5,8,11-tetraoxatridecan-2-yl 4-dodecyl-3-fluoro-1H-pyrrole-2-carboxylate;
Ethyl 3-fluoro-4-tridecyl-1H-pyrrole-2-carboxylate;
3-Fluoro-4-tridecyl-1H-pyrrole-2-carboxylic acid;
Methyl 3-fluoro-4-tridecyl-1H-pyrrole-2-carboxylate;
Isopropyl 3-fluoro-4-tridecyl-1H-pyrrole-2-carboxylate;
Tert-butyl 3-fluoro-4-tridecyl-1H-pyrrole-2-carboxylate;
Cyclohexyl 3-fluoro-4-tridecyl-1H-pyrrole-2-carboxylate;
Benzyl 3-fluoro-4-tridecyl-1H-pyrrole-2-carboxylate;
2,2,2-Trifluoroethyl 3-fluoro-4-tridecyl-1H-pyrrole-2-carboxylate;
2-(2,5-Dioxopyrrolidin-1-yl)ethyl 3-fluoro-4-tridecyl-1H-pyrrole-2-carboxylate;
2-(2-Oxopyrrolidin-1-yl)ethyl 3-fluoro-4-tridecyl-1H-pyrrole-2-carboxylate;
(5-Methyl-2-oxo-1,3-dioxol-4-yl)methyl 3-fluoro-4-tridecyl-1H-pyrrole-2-carboxylate;
2-((2-Ethoxy-2-oxoethyl)(methyl)amino)-2-oxoethyl 3-fluoro-4-tridecyl-1H-pyrrole;
2-carboxylate;
2-Hydroxyethyl 3-fluoro-4-tridecyl-1H-pyrrole-2-carboxylate;
3-Hydroxypropyl 3-fluoro-4-tridecyl-1H-pyrrole-2-carboxylate;
4-Hydroxybutyl 3-fluoro-4-tridecyl-1H-pyrrole-2-carboxylate;
5-Hydroxypentyl 3-fluoro-4-tridecyl-1H-pyrrole-2-carboxylate;
6-Hydroxyhexyl 3-fluoro-4-tridecyl-1H-pyrrole-2-carboxylate;
7-Hydroxyheptyl 3-fluoro-4-tridecyl-1H-pyrrole-2-carboxylate;
8-Hydroxyoctyl 3-fluoro-4-tridecyl-1H-pyrrole-2-carboxylate;
9-Hydroxynonyl 3-fluoro-4-tridecyl-1H-pyrrole-2-carboxylate;
2,3-Dihydroxypropyl 3-fluoro-4-tridecyl-1H-pyrrole-2-carboxylate;
1,3-Dihydroxypropan-2-yl 3-fluoro-4-tridecyl-1H-pyrrole-2-carboxylate;
2-(2-(2-(2-Hydroxyethoxy)ethoxy)ethoxy)ethyl 3-fluoro-4-tridecyl-1H-pyrrole-2-carboxylate;
2-(2-Ethoxyethoxy)ethyl 3-fluoro-4-tridecyl-1H-pyrrole-2-carboxylate;
1-((Isopropoxycarbonyl)oxy)ethyl 3-fluoro-4-tridecyl-1H-pyrrole-2-carboxylate;
1-((Tert-butoxycarbonyl)oxy)ethyl 3-fluoro-4-tridecyl-1H-pyrrole-2-carboxylate;
1-(((Nonyloxy)carbonyl)oxy)ethyl 3-fluoro-4-tridecyl-1H-pyrrole-2-carboxylate;
1-(((Cyclohexyloxy)carbonyl)oxy)ethyl 3-fluoro-4-tridecyl-1H-pyrrole-2-carboxylate;
1-(((Benzyloxy)carbonyl)oxy)ethyl 3-fluoro-4-tridecyl-1H-pyrrole-2-carboxylate;
1-(((2-Methoxyethoxy)carbonyl)oxy)ethyl 3-fluoro-4-tridecyl-1H-pyrrole-2-carboxylate;
1-(((3-Hydroxypropoxy)carbonyl)oxy)ethyl 3-fluoro-4-tridecyl-1H-pyrrole-2-carboxylate;
4-Oxo-3,5,8,11-tetraoxatridecan-2-yl 3-fluoro-4-tridecyl-1H-pyrrole-2-carboxylate;
3-Fluoro-4-tetradecyl-1H-pyrrole-2-carboxylic acid;
3-Fluoro-4-pentadecyl-1H-pyrrole-2-carboxylic acid;
3-Fluoro-4-heptadecyl-1H-pyrrole-2-carboxylic acid;
5-Dodecyl-3-fluoro-1H-pyrrole-2-carboxylic acid;
3-Chloro-4-decyl-1H-pyrrole-2-carboxylic acid;
3-Chloro-4-undecyl-1H-pyrrole-2-carboxylic acid;
3-Chloro-4-dodecyl-1H-pyrrole-2-carboxylic acid;
9-Hydroxynonyl 3-chloro-4-dodecyl-1H-pyrrole-2-carboxylate;
2-(2,5-dioxopyrrolidin-1-yl)ethyl 3-chloro-4-dodecyl-1H-pyrrole-2-carboxylate;
3-Chloro-4-tridecyl-1H-pyrrole-2-carboxylic acid;
3-Chloro-4-pentadecyl-1H-pyrrole-2-carboxylic acid;
3-Chloro-4-hexadecyl-1H-pyrrole-2-carboxylic acid;
3-Chloro-5-undecyl-1H-pyrrole-2-carboxylic acid;
3-Chloro-5-dodecyl-1H-pyrrole-2-carboxylic acid;
3-Chloro-5-tridecyl-1H-pyrrole-2-carboxylic acid;
3-chloro-5-tetradecyl-1H-pyrrole-2-carboxylic acid;
3-Bromo-4-tridecyl-1H-pyrrole-2-carboxylic acid;
1-Butyl-3-fluoro-4-tridecyl-1H-pyrrole-2-carboxylic acid;
3-Fluoro-1-isopropyl-4-tridecyl-1H-pyrrole-2-carboxylic acid;
4-(Decyloxy)-3-fluoro-1H-pyrrole-2-carboxylic acid;
3-Fluoro-4-(undecyloxy)-1H-pyrrole-2-carboxylic acid;
4-(Dodecyloxy)-3-fluoro-1H-pyrrole-2-carboxylic acid;
3-Fluoro-4-(tridecyloxy)-1H-pyrrole-2-carboxylic acid;
3-Fluoro-4-(tetradecyloxy)-1H-pyrrole-2-carboxylic acid;
4-(Dodecylthio)-3-fluoro-1H-pyrrole-2-carboxylic acid;
3-Chloro-4-(nonyloxy)-1H-pyrrole-2-carboxylic acid;
3-Chloro-4-(decyloxy)-1H-pyrrole-2-carboxylic acid;
3-chloro-4-(undecyloxy)-1H-pyrrole-2-carboxylic acid;
3-Chloro-4-(dodecyloxy)-1H-pyrrole-2-carboxylic acid;
2,2,2-trifluoroethyl 3-chloro-4-(dodecyloxy)-1H-pyrrole-2-carboxylate;
9-hydroxynonyl 3-chloro-4-(dodecyloxy)-1H-pyrrole-2-carboxylate;
2-(2-ethoxyethoxy)ethyl 3-chloro-4-(dodecyloxy)-1H-pyrrole-2-carboxylate;
2,3-Dihydroxypropyl 3-chloro-4-(dodecyloxy)-1H-pyrrole-2-carboxylate;
1-((isopropoxycarbonyl)oxy)ethyl 3-chloro-4-(dodecyloxy)-1H-pyrrole-2-carboxylate;
4-Oxo-3,5,8,11-tetraoxatridecan-2-yl 3-chloro-4-(dodecyloxy)-1H-pyrrole-2-carboxylate;
1-(((3-hydroxypropoxy)carbonyl)oxy)ethyl 3-chloro-4-(dodecyloxy)-1H-pyrrole-2-carboxylate;
(5-methyl-2-oxo-1,3-dioxol-4-yl)methyl 3-chloro-4-(dodecyloxy)-1H-pyrrole-2-carboxylate;
3-Chloro-4-(tridecyloxy)-1H-pyrrole-2-carboxylic acid;
3-Chloro-4-(tetradecyloxy)-1H-pyrrole-2-carboxylic acid;
3-Fluoro-4-pentadecanoyl-1H-pyrrole-2-carboxylic acid;
4-(12-Ethoxydodecyl)-3-fluoro-1H-pyrrole-2-carboxylic acid;
3-fluoro-4-(2-fluorotridecyl)-1H-pyrrole-2-carboxylic acid;
4-(2,2-Difluorotridecyl)-3-fluoro-1H-pyrrole-2-carboxylic acid;
4-(3,3-dimethyldodecyl)-3-fluoro-1H-pyrrole-2-carboxylic acid;
4-((2,2-dimethyltridecyl)oxy)-3-fluoro-1H-pyrrole-2-carboxylic acid;
4-((2,2-difluorotetradecyl)oxy)-3-fluoro-1H-pyrrole-2-carboxylic acid;
4-((2,2-difluoroundecyl)oxy)-3-fluoro-1H-pyrrole-2-carboxylic acid;
3-chloro-4-((2-fluorotetradecyl)oxy)-1H-pyrrole-2-carboxylic acid;
3-chloro-4-((9-ethoxynonyl)oxy)-1H-pyrrole-2-carboxylic acid;
3-Methyl-4-tridecyl-1H-pyrrole-2-carboxylic acid;
4-(2,2-Dimethyldodecyl)-3-fluoro-1H-pyrrole-2-carboxylic acid;
2,2,2-Trifluoroethyl 3-fluoro-4-(undecyloxy)-1H-pyrrole-2-carboxylate;
2-(2-Ethoxyethoxy)ethyl 3-fluoro-4-(undecyloxy)-1H-pyrrole-2-carboxylate;
1-((Isopropoxycarbonyl)oxy)ethyl 3-fluoro-4-(undecyloxy)-1H-pyrrole-2-carboxylate;
1-(((2-Methoxyethoxy)carbonyl)oxy)ethyl 3-fluoro-4-(undecyloxy)-1H-pyrrole-2-carboxylate;
4-Oxo-3,5,8,11-tetraoxatridecan-2-yl 3-fluoro-4-(undecyloxy)-1H-pyrrole-2-carboxylate;
2,2,2-Trifluoroethyl 3-chloro-4-tridecyl-1H-pyrrole-2-carboxylate;
2-(2-Ethoxyethoxy)ethyl 3-chloro-4-tridecyl-1H-pyrrole-2-carboxylate;
1-((Isopropoxycarbonyl)oxy)ethyl 3-chloro-4-tridecyl-1H-pyrrole-2-carboxylate;
1-(((2-methoxyethoxy)carbonyl)oxy)ethyl 3-chloro-4-tridecyl-1H-pyrrole-2-carboxylate;
4-Oxo-3,5,8,11-tetraoxatridecan-2-yl 3-chloro-4-tridecyl-1H-pyrrole-2-carboxylate;
2,2,2-trifluoroethyl 3-chloro-5-dodecyl-1H-pyrrole-2-carboxylate;
2-(2-ethoxyethoxy)ethyl 3-chloro-5-dodecyl-1H-pyrrole-2-carboxylate;
1-((isopropoxycarbonyl)oxy)ethyl 3-chloro-5-dodecyl-1H-pyrrole-2-carboxylate;
1-(((2-methoxyethoxy)carbonyl)oxy)ethyl 3-chloro-5-dodecyl-1H-pyrrole-2-carboxylate;
4-oxo-3,5,8,11-tetraoxatridecan-2-yl 3-chloro-5-dodecyl-1H-pyrrole-2-carboxylate;
2,3-dihydroxypropyl 3-chloro-5-dodecyl-1H-pyrrole-2-carboxylate;
3-Fluoro-5-undecyl-1H-pyrrole-2-carboxylic acid;
3-Fluoro-5-tridecyl-1H-pyrrole-2-carboxylic acid;
3-Fluoro-5-tetradecyl-1H-pyrrole-2-carboxylic acid;
3-Fluoro-5-pentadecyl-1H-pyrrole-2-carboxylic acid;
3-Fluoro-5-hexadecyl-1H-pyrrole-2-carboxylic acid;
3-Fluoro-5-heptadecyl-1H-pyrrole-2-carboxylic acid;
3-Fluoro-5-octadecyl-1H-pyrrole-2-carboxylic acid;
3-Fluoro-5-octadecyl-1H-pyrrole-2-carboxylic acid;
3-Chloro-5-(2,2-dimethyldodecyl)-1H-pyrrole-2-carboxylic acid;
3-Chloro-5-(3,3-difluorododecyl)-1H-pyrrole-2-carboxylic acid;
3-Cyano-5-dodecyl-1H-pyrrole-2-carboxylic acid;
3-Chloro-5-dodecyl-1-methyl-1H-pyrrole-2-carboxylic acid;
3-Fluoro-5-(14-fluorotetradecyl)-1H-pyrrole-2-carboxylic acid;
3-Fluoro-4-hexadecyl-1H-pyrrole-2-carboxylic acid;
or a pharmaceutically acceptable salt, or solvate, or N-oxide, or stereoisomer, or tautomer, or isotopically labelled derivative thereof.

13. A method for treating a subject afflicted with a pathological condition or disease susceptible to amelioration by inhibition of Acetyl-CoA carboxylase, the method comprising administering to the subject an effective amount of a compound according to claim 1.

14. The method according to claim 13, wherein the pathological condition or disease is chosen from acne vulgaris, acne conglobata, inflammatory acne, choracne, rosacea, Rhinophyma-type rosacea, seborrhea, seborrheic dermatitis, sebaceous gland hyperplasia, Meibomian gland dysfunction of facial rosacea, mitogenic alopecia, oily skin, plaque psoriasis, guttate psoriasis, inverse psoriasis, erythrodermic psoriasis, scalp psoriasis, nail psoriasis, postular psoriasis, and palmoplantar pustulosis.

15. The method according to claim 14, wherein the treatment is of a pathological condition or disease is chosen from acne vulgaris, acne conglobata, inflammatory acne, choracne, plaque psoriasis, guttate psoriasis, inverse psoriasis, erythrodermic psoriasis, scalp psoriasis, nail psoriasis and postular psoriasis.

16. A pharmaceutical composition comprising a compound according to claim 1 and a pharmaceutically acceptable diluent or carrier.

17. (canceled)

18. A method for treating a subject afflicted with a pathological condition or disease susceptible to amelioration by inhibition of Acetyl-CoA carboxylase, the method comprising administering to the subject a pharmaceutical composition according to claim 16.

19. A combination product comprising (i) at least one compound according to claim 1, and (ii) one or more active ingredients chosen from:
a) Corticoids and glucocorticoids;
b) Dihydrofolate reductase inhibitors;
c) Dihydroorotate dehydrogenase (DHODH) inhibitors;
d) Purine antagonists;
e) Antimalarials;
f) Calcineurin inhibitors;
g) Inosine-monophosphate dehydrogenase (IMPDH) inhibitors;
h) Fumaric acid esters;
i) Vitamin D3 derivatives;
j) Retinoids;
k) Anti-tumor necrosis factor-alpha (Anti-TNF-alpha) monoclonal antibodies;
l) Soluble Tumor necrosis factor-alpha (TNF-alpha) receptors;
m) Anti-Interleukin 6 Receptor (IL-6R) antibody;
n) Anti-Interleukin 12 (IL-12)/Interleukin 23 (IL-23) antibody;
o) Anti-Interleukin 17 Receptor (IL-17R) antibody;
p) Anti-CD20 (B lymphocyte protein) antibody;
q) Anti-Interleukin 5 (IL-5) antibody;
r) Anti-Interleukin 5 Receptor (IL-5R) antibody;
s) Anti-Interleukin 13 (IL-13) antibody;
t) Anti-Interleukin 4 Receptor (IL-4R)/Interleukin 13 Receptor (IL-13R) antibody;
u) Anti-Interleukin 17 (IL-17) antibody;
v) An anti-IL-23 antibody;
w) Anti-Interleukin 1 Receptor (IL-1R) antibody;
x) Anti-Immunoglobulin E (IgE) antibody;
y) Anti-B-cell activating factor (BAFF);
z) Anti-CD19 (B lymphocyte protein) monoclonal antibody;
aa) Kappa opioid agonists;
bb) Neurokinin receptor 1 antagonists;
cc) Dihydropteroate synthase inhibitors;
dd) Histamine 1 (H1) receptor antagonists;
ee) Cysteinyl leukotriene (CysLT) receptor antagonists;
ff) Chemoattractant receptor homologous molecule expressed on TH2 cells (CRTh2) antagonists;
gg) Topical anti-septics;
hh) Antibiotics;
ii) Azelaic acid;
jj) α-hydroxy acids such as glycolic acid or lactic acid;
kk) β-hydroxy acids; and
A PDE4 inhibitor.
